(19) United States
(12) Patent Application Publication
(10) Pub. No.: US 2023/0201316 A1
(43) Pub. Date: Jun. 29, 2023

(54) ATP-HYDROLYZING ENZYME USEFUL FOR TREATING DYSBIOSIS

(71) Applicant: MV BIOTHERAPEUTICS SA, Bellinzona (CH)

(72) Inventors: Fabio GRASSI, Bellinzona (CH); Lisa PERRUZZA, Bellinzona (CH)

(21) Appl. No.: 17/999,460

(22) PCT Filed: Jun. 8, 2021
(86) PCT No.: PCT/EP2021/065315

§ 371 Date: Nov. 21, 2022

(30) Foreign Application Priority Data
Jun. 8, 2020 (WO) PCT/EP2020/065849

(51) Int. Cl. A61K 38/46; A61K 35/741; A61P 1/14; A61P 3/00; A61K 35/744; A61P 31/04; A61K 31/546

(52) U.S. Cl. CPC A61K 38/46; A61K 35/741; A61P 1/14; A61P 3/00; A61K 35/744; A61P 31/04; A61K 31/546; C12Y 306/01005; A61K 2035/115

(57) ABSTRACT

The present invention provides an ATP hydrolyzing enzyme, a nucleic acid encoding an ATP hydrolyzing enzyme, or host cells, microorganisms, such as bacteria, or viral particles comprising such nucleic acids encoding an ATP hydrolyzing enzyme for use in the treatment of dysbiosis or a dysbiosis-related disease.

The present invention relates to the treatment of dysbiosis, and to agents useful in the treatment of dysbiosis.

The human gastrointestinal (GI) tract is a complex ecological niche, in which all the three domains of life (Archaea, Bacteria and Eukarya) and Viruses co-exist in close association with the host (Arumugam, M., Raes, J., Pelletier, E., Le Paslier, D., Yamada, T., Mende, D. R., Fernandes, G. R., Tap, J., Bruls, T., Batto, J. M., et al. (2011). Enterotypes of the human gut microbiome. Nature 473, 174-180; Human Microbiome Project, C. (2012). Structure, function and diversity of the healthy human microbiome. Nature 486, 207-214; Reyes, A., Haynes, M., Hanson, N., Angly, F. E., Heath, A. C., Rohwer, F., and Gordon, J. I. (2010). Viruses in the faecal microbiota of monozygotic twins and their mothers. Nature 466, 334-338). This complex microbial community, referred to as the gut microbiota, has co-evolved with the host in a mutualistic relationship that influences many physiological functions such as energy harvesting, development and function of the immune system (Kamada, N., Seo, S. U., Chen, G. Y., and Nunez, G. (2013). Role of the gut microbiota in immunity and inflammatory disease. Nat Rev Immunol 13, 321-335; Maslowski, K. M., and Mackay, C. R. (2011). Diet, gut microbiota and immune responses. Nat Immunol 12, 5-9; Sampson, T. R., and Mazmanian, S. K. (2015). Control of brain development, function, and behavior by the microbiome. Cell host & microbe 17, 565-576). The subtle equilibrium between the gut microbiota and the host is a key element in human health. In fact, alterations in the composition of the microbial community structure, termed dysbiosis, have been associated to an increasing number of medical conditions such as metabolic disorders (e.g. diabetes, obesity) (Holmes, E., Loo, R. L., Stamler, J., Bictash, M., Yap, I. K., Chan, Q., Ebbels, T., De Iorio, M., Brown, I. J., Veselkov, K. A., et al. (2008). Human metabolic phenotype diversity and its association with diet and blood pressure. Nature 453, 396-400; Larsen, N., Vogensen, F. K., van den Berg, F. W., Nielsen, D. S., Andreasen, A. S., Pedersen, B. K., Al-Soud, W. A., Sorensen, S. J., Hansen, L. H., and Jakobsen, M. (2010). Gut microbiota in human adults with type 2 diabetes differs from non-diabetic adults. PLoS One 5, e9085; Turnbaugh, P. J., Hamady, M., Yatsunenko, T., Cantarel, B. L., Duncan, A., Ley, R. E., Sogin, M. L., Jones, W. J., Roe, B. A., Affourtit, J. P., et al. (2009). A core gut microbiome in obese and lean twins. Nature 457, 480-484), blood pressure alteration and heart disease (Blaser, M. J. (2006). Who are we? Indigenous microbes and the ecology of human diseases. EMBO Rep 7, 956-960), and autoimmunity (Dicksved, J., Halfvarson, J., Rosenquist, M., Jarnerot, G., Tysk, C., Apajalahti, J., Engstrand, L., and Jansson, J. K. (2008). Molecular analysis of the gut microbiota of identical twins with Crohn's disease. ISME J 2, 716-727).

More than 90% of pathogens infect humans through mucosal surfaces (Brandtzaeg, P. (2010). The mucosal immune system and its integration with the mammary glands. J Pediatr 156, S8-15). The intestinal microbiota provides resistance to infectious diseases through four mechanisms: direct inhibition, barrier maintenance, immune modulation, and bacterial metabolism (McKenney, P. T., and Pamer, E. G. (2015). From Hype to Hope: The Gut Microbiota in Enteric Infectious Disease. Cell 163, 1326-1332). Collectively, these mechanisms configure “colonization resistance” (Lawley, T. D., and Walker, A. W. (2013). Intestinal colonization resistance. Immunology 138, 1-11). The importance of colonization resistance in protection from enteric infections is exemplified by a 100,000-fold decrease in the dose of Salmonella Typhimurium required to infect mice treated with antibiotics (Bohnhoff, M., Drake, B. L., and Miller, C. P. (1954). Effect of streptomycin on susceptibility of intestinal tract to experimental Salmonella infection. Proc Soc Exp Biol Med 86, 132-137).

A balanced structure of the intestinal microbiome is of fundamental importance for the metabolic homeostasis of the host. Different studies in mice and humans have demonstrated that obesity is associated with changes in microbiota's diversity and abundance. Furthermore, it has been suggested that intestinal dysbiosis has a causal role in the development of obesity and insulin resistance. Indeed, faecal microbiota transfer (FMT) from conventional to germ-free (GF) mice results in significant increase in body-fat content and insulin resistance (Turnbaugh, P. J., Ley, R. E., Mahowald, M. A., Magrini, V., Mardis, E. R., and Gordon, J. I. (2006). An obesity-associated gut microbiome with increased capacity for energy harvest. Nature 444, 1027-1031) that are associated with inflammation and macrophage accumulation in adipose tissue (Caesar, R., Reigstad, C. S., Backhed, H. K., Reinhardt, C., Ketonen, M., Lunden, G. O., Cani, P. D., and Backhed, F. (2012). Gut-derived lipopolysaccharide augments adipose macrophage accumulation but is not essential for impaired glucose or insulin tolerance in mice. Gut 61, 1701-1707). The gut microbiota encodes a more versatile metabolome than the host and a healthy microbiota is a necessary requirement for stable functional metabolic interactions with the host.

By modulating AMP protein kinase (AMPK) activity in the liver and its downstream targets involved in fatty acid oxidation (Backhed, F., Manchester, J. K., Semenkovich, C. F., and Gordon, J. I. (2007). Mechanisms underlying the resistance to diet-induced obesity in germ-free mice. Proc Natl Acad Sci USA 104, 979-984), the gut microbiota promotes glucose uptake in the small intestine as well as generation of short chain fatty acid (SCFA) in the distal gut (Wolin, M. J. (1981). Fermentation in the rumen and human large intestine. Science 213, 1463-1468). Dysbiosis, e.g. induced by antibiotic treatments, decreases serum glucose levels and improves insulin sensitivity. Extensive tissue remodeling and decreased availability of short chain fatty acids (SCFAs) alters glucose homeostasis by shifting colonocyte energy utilization from SCFAs to glucose (Zarrinpar, A., Chaix, A., Xu, Z. Z., Chang, M. W., Marotz, C. A., Saghatelian, A., Knight, R., and Panda, S. (2018). Antibiotic-induced microbiome depletion alters metabolic homeostasis by affecting gut signaling and colonic metabolism. Nat Commun 9, 2872). Moreover, trimethylamine N-oxide (TMAO) levels, a gut microbiota-dependent metabolite, and the related N-oxide (TMAO)-generating pathway, are linked to obesity and energy metabolism (Org, E., Blum, Y., Kasela, S., Mehrabian, M., Kuusisto, J., Kangas, A. J., Soininen, P., Wang, Z., Ala-Korpela, M., Hazen, S. L., et al. (2017). Relationships between gut microbiota, plasma metabolites, and metabolic syndrome traits in the METSIM cohort. Genome Biol 18, 70; Schugar, R. C., Shih, D. M., Warrier, M., Helsley, R. N., Burrows, A., Ferguson, D., Brown, A. L., Gromovsky, A. D., Heine, M., Chatterjee, A., et al. (2017). The TMAO-Producing Enzyme Flavin-Containing Monooxygenase 3 Regulates Obesity and the Beiging of White Adipose Tissue. Cell Rep 19, 2451-2461), underscoring the importance of the gut microbiota in the aetiology of metabolic disorders.

In summary, dysbiosis of human microbiota is associated with a number of diseases. Therefore, several options have been studied to prevent or treat dysbiosis. For a long time, antibiotics were administered to select which bacteria to preserve in the intestinal ecosystem (Sanders, W. E., Jr., Sanders, C. C.: Modification of normal flora by antibiotics: effects on individuals and the environment. In: Koot, R. K., Sande, M. A. (ed.): New dimensions in antimicrobial therapy. Churchill Livingstone, N.Y., 1984, p. 217-241). However, this strategy is now being actively avoided because of its selection pressure and the risks of emergence of antibiotic-resistant bacteria. Other strategies include fecal microbiota transplants (FMTs), which are currently used to treat patients with Clostridium difficile infections, who have proved resistant to other therapies (Smith M B, Kelly C, Alm E J (February 2014). “Policy: How to regulate faecal transplants”. Nature. 506 (7488): 290-291. doi:10.1038/506290a). However, this therapy is still investigational and there are concerns in particular because the process is not sterile and contaminations can pass from donor to patient. In view thereof, probiotics and prebiotics recently emerged as promising tools for the treatment of dysbiosis. However, under certain circumstances, their efficacy may be limited.

In view of the above, it is the object of the present invention to overcome the drawbacks of the prior art and to provide novel agents useful in the treatment or prevention of dysbiosis and/or dysbiosis-related diseases.

This object is achieved by means of the subject-matter set out below and in the appended claims.

Although the present invention is described in detail below, it is to be understood that this invention is not limited to the particular methodologies, protocols and reagents described herein as these may vary. It is also to be understood that the terminology used herein is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded. In the context of the present invention, the term “comprise” encompasses the term “consist of”. The term “comprising” thus encompasses “including” as well as “consisting” e.g., a composition “comprising” X may consist exclusively of X or may include something additional e.g., X+Y.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention.

The word “substantially” does not exclude “completely” e.g., a composition which is “substantially free” from Y may be completely free from Y. Where necessary, the word “substantially” may be omitted from the definition of the invention.

The term “about” in relation to a numerical value x means x±10%.

ATP-Hydrolyzing Enzyme for the Treatment of Dysbiosis

In a first aspect the present invention provides
(a) an ATP hydrolyzing enzyme,
(b) a nucleic acid comprising a polynucleotide encoding the ATP hydrolyzing enzyme,
(c) a host cell comprising the nucleic acid,
(d) a microorganism comprising the nucleic acid, or
(e) a viral particle comprising the nucleic acid

for use in the treatment of dysbiosis or a dysbiosis-related disease.

In particular, the present invention provides an ATP hydrolyzing enzyme, a nucleic acid comprising a polynucleotide encoding the ATP hydrolyzing enzyme, a host cell comprising the nucleic acid, a microorganism comprising the nucleic acid, a (recombinant) bacterium comprising the nucleic acid, or a viral particle comprising the nucleic acid

for use in restoring or improving the microbiome balance during or after dysbiosis, for example dysbiosis induced by a dysbiosis-inducing agent as described herein.

The present inventors surprisingly found that administration of an ATP-hydrolyzing enzyme (or of a host cell/microorganism comprising a nucleic acid encoding the ATP-hydrolyzing enzyme) effectively counteracts (induction of) dysbiosis. In particular, enhanced intestinal microbial diversity, blooming of beneficial microbes, maintenance/restoration of colonization resistance and metabolic improvement was observed, as shown in the appended examples. Moreover, the experimental data show that symptoms and diseases related to dysbiosis are effectively treated in vivo by administration of an ATP-hydrolyzing enzyme (or of a host cell/microorganism comprising a nucleic acid encoding the ATP-hydrolyzing enzyme). Accordingly, administration of an ATP-hydrolyzing enzyme—or a nucleic acid encoding an ATP-hydrolyzing enzyme; or a host cell, microorganism or viral particle comprising such a nucleic acid (and, thus, expressing an ATP-hydrolyzing enzyme)—can effectively prevent or treat dysbiosis and diseases related to (driven by) dysbiosis.

Dysbiosis and Dysbiosis-Related Diseases

The ATP-hydrolyzing enzyme—or the nucleic acid encoding the ATP-hydrolyzing enzyme; or the host cell, microorganism or viral particle comprising such a nucleic acid (and, thus, expressing an ATP-hydrolyzing enzyme)—is used (for the preparation of a medicament) for the treatment of dysbiosis or a dysbiosis-related disease.

The term “dysbiosis”, as used herein, refers to an abnormal microbiome structure, which affects the taxonomical composition as well as the metagenomic function of the microbial community. Accordingly, dysbiosis is an imbalance in the composition of microbiota, in particular of human microbiota. As used herein, the term “microbiota” refers to commensal microorganisms found in and on all multicellular organisms studied to date from plants to animals. In particular, microbiota have been found to be crucial for immunologic, hormonal and metabolic homeostasis of their host. In particular, microbiota are non-pathogenic. In other words, microbiota (in their normal, balanced composition) are usually not (capable of) causing a disease in the host and/or they are not harmful to the host. Accordingly, the interaction between microbiota and their host is usually commensal or symbiotic. Microbiota include bacteria, archaea, protists, fungi, viruses and phages. Anatomically, microbiota reside on or within any of a number of tissues and biofluids, including the gastrointestinal tract (GI), in particular the gut (and the oral cavity, in particular the oral mucosa), skin, conjunctiva, mammary glands, vagina, placenta, seminal fluid, uterus, ovarian follicles, lung and saliva. Dysbiosis is most commonly reported as a condition in the gastrointestinal tract, for example during small intestinal bacterial overgrowth (SIBO) or small intestinal fungal overgrowth (SIFO). In some embodiments, the microbiota are gastrointestinal tract (GI) microbiota and the dysbiosis is, thus, gastrointestinal dysbiosis. GI microbiota may be selected from gut microbiota and oral cavity microbiota and, therefore, GI dysbiosis may be selected from gut dysbiosis and oral cavity dysbiosis. Intestinal dysbiosis is the disturbance of the normal balance of microbiota species in the intestine. Symptoms of intestinal dysbiosis include upset stomach, nausea, constipation, diarrhea, and bloating.

Dysbiosis can be induced by various factors including external factors (such as administration of antibiotics or chemotherapeutic agents, some substances introduced in diet, physical and psychological stress) and host-related factors. Major causes of dysbiosis include dietary disorders (a hyperprotein hyperlipid diet, rich in sugars and low in fiber; food allergies; malabsorption and impaired digestion of carbohydrates), poor digestive secretions, stress, antibiotic/pharmacological therapy, weakened immune functions, malabsorption, intestinal infections and alterations of the pH in the gastrointestinal tract. In some embodiments, the dysbiosis is induced by antibiotics. In other embodiments, the dysbiosis is induced by chemotherapeutic agents.

In summary, “dysbiosis” refers to the disruption of the balance of the microbiota and, consequently, its normal functioning. This results in the selective suppression of some species in the microbiota leading to unregulated production of microorganism-derived products or metabolites that can become dangerous for the host, to the point of causing various disorders locally, systemically or even in more distant organs. Accordingly, dysbiosis is an abnormal microbial ecological state that is causally linked to the manifestation of various diseases.

In dysbiosis, normally dominating microbiota species become underrepresented, while normally outcompeted or contained species may increase to fill the void. As normally dominating microbiota species are usually benign or beneficial and carry out a series of helpful and necessary functions, such as aiding in digestion and providing protection from pathogenic microbes, the selective suppression of beneficial microbiota species in dysbiosis leads to unregulated production of microorganism-derived products or metabolites that can become dangerous for the host, to the point of causing various disorders locally, systemically or even in more distant organs. Accordingly, dysbiosis can trigger the onset of chronic disease in various ways. For example, pathogens and their functions can be acquired or opportunistically overgrow in dysbiosis, which results in infectious diseases such as cholera or streptococcal pharyngitis, but which can also lead to chronic inflammation. Moreover, health-protective bacteria and their functions may be lost or suppressed, which then promotes the onset of diseases, in particular chronic diseases such as inflammatory bowel disease (IBD), urinary stone disease (USD), obesity, and others.

Various diseases are known to be related to dysbiosis including inflammatory diseases, gastrointestinal tract-related disorders, metabolic disorders, CNS-related disorders, cancers and autoimmune diseases. Accordingly, the dysbiosis-related disease may be selected from inflammatory diseases, infectious diseases, gastrointestinal tract-related disorders, metabolic disorders, CNS-related disorders, cancers and autoimmune diseases.

Non-limiting examples of inflammatory diseases include pancreatitis, gingivitis, periodontitis, inflammatory bowel disease (IBD), Crohn's disease (CD), ulcerative colitis (UC), gastritis, enteritis, esophagitis, diverticulitis, rheumatoid arthritis and infectious colitis.

Non-limiting examples of infectious diseases include a gastrointestinal infection, a respiratory infection, a kidney infection, and infections with specific pathogens, such as Clostridioides difficile infection and Citrobacter rodentium infection.

Non-limiting examples of gastrointestinal tract-related disorders and metabolic disorders include inflammatory bowel disease (IBD), Crohn's disease (CD), ulcerative colitis (UC), gastritis, enteritis, esophagitis, gastroesophageal reflux disease (GERD), celiac disease, ulcer, irritable bowel syndrome, obesity, diabetes, and metabolic syndrome.

In general, non-limiting examples of diseases induced by or associated with dysbiosis include inflammatory bowel disease, irritable bowel syndrome, obesity, diabetes, metabolic syndrome, coeliac disease, colorectal cancer, Clostridioides difficile infection, autism spectrum disorder, urinary stone disease (USD), lupus erythematosus, rheumatoid arthritis, systemic sclerosis, Sjögren's syndrome, anti-phospholipid syndrome, cardiovascular syndrome, allergy, and asthma. Accordingly, the dysbiosis-related disease may be selected from inflammatory bowel disease, irritable bowel syndrome, obesity, diabetes, metabolic syndrome, coeliac disease, colorectal cancer, Clostridioides difficile infection, autism spectrum disorder, urinary stone disease (USD), lupus erythematosus, rheumatoid arthritis, systemic sclerosis, Sjögren's syndrome, anti-phospholipid syndrome, cardiovascular syndrome, allergy, and asthma.

In some embodiments, the dysbiosis-related disease is irritable bowel syndrome (IBS) or inflammatory bowel disease (IBD), such as Crohn's disease (CD) and/or ulcerative colitis (UC). IBD is a group of inflammatory conditions of the colon and small intestine, which includes Crohn's disease and ulcerative colitis. IBD-affected individuals have been found to have 30-50 percent reduced biodiversity of commensal bacteria, such as decreases in Firmicutes (namely Lachnospiraceae) and Bacteroidetes.

The “treatment” of dysbiosis or a dysbiosis-related disease may be a prophylactic treatment (e.g., reducing the risk of occurrence) or a therapeutic treatment. As used herein, the term “therapeutic treatment” refers to treatment after the onset of dysbiosis or a dysbiosis-related disease, while “prophylactic treatment” refers to treatment before the onset of dysbiosis or a dysbiosis-related disease or before the first symptoms occur. In particular, “therapeutic treatment” does not include prophylactic measures applied before the onset of dysbiosis or a dysbiosis-related disease. Since the onset of dysbiosis or a dysbiosis-related disease is often associated with symptom(s) of dysbiosis or the dysbiosis-related disease, human or animal subjects are often “therapeutically” treated after the diagnosis or at least a (strong) assumption that the subject suffers from certain dysbiosis or a dysbiosis-related disease. Therapeutic treatment aims in particular at (1) ameliorating, reducing, improving, or curing a disease (state) or (2) at inhibiting or delaying the progression of a disease. However, prevention of the onset of a disease cannot typically be achieved by therapeutic treatment. Prophylactic treatment includes reducing the risk of occurrence of dysbiosis or a dysbiosis-related disease or reducing the degree of dysbiosis or a dysbiosis-related disease (when it occurs) in a prophylactic manner. For example, (prophylactic or therapeutic) treatment of dysbiosis may be considered as prophylactic treatment of diseases induced by dysbiosis.

As used herein, the term “disease” is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

ATP-Hydrolyzing Enzymes and Nucleic Acids Encoding ATP-Hydrolyzing Enzymes

According to a first aspect of the present invention, an ATP-hydrolyzing enzyme may be used for the treatment of dysbiosis or a dysbiosis-related disease.

As used herein, the term “ATP-hydrolyzing enzyme” refers to any enzyme which catalyzes the hydrolysis of ATP to ADP, ATP to AMP and/or ADP to AMP. Such enzymes include but are not limited to apyrase, ATPase, ATP-diphosphatase, adenosine diphosphatase, ADPase, ATP-diphosphohydrolase and CD39 (Ectonucleoside triphosphate diphosphohydrolase 1, ENTPD1). In the context of the present invention, any ATP-hydrolyzing enzyme may be used.

In some embodiments, the ATP-hydrolyzing enzyme is not endogenous CD39 (Ectonucleoside triphosphate diphosphohydrolase 1, ENTPD1). Endogenous CD39 is an integral membrane protein that hydrolyses ATP and ADP in a calcium and magnesium dependent reaction generating AMP. It is activated upon glycosylation and translocation to the cell surface membrane where it displays its enzyme activity as an ectonucleotidase. CD39 is attached to the plasma membrane by two transmembrane domains (Grinthal A, Guidotti G. CD39, NTPDase 1, is attached to the plasma membrane by two transmembrane domains. Why?. Purinergic Signal. 2006; 2(2):391-398. doi:10.1007/s11302-005-5907-8). However, as described below, in the context of the present invention soluble (not membrane-bound) ATP-hydrolyzing enzymes are preferred. In contrast to membrane-bound endogenous CD39, CD39 can be engineered to obtain a soluble form of CD39 (Gayle R B 3rd, Maliszewski C R, Gimpel S D, Schoenborn M A, Caspary R G, Richards C, Brasel K, Price V, Drosopoulos J H, Islam N, Alyonycheva T N, Broekman M J, Marcus A J. Inhibition of platelet function by recombinant soluble ecto-ADPase/CD39. J Clin Invest. 1998 May 1; 101(9):1851-9. doi: 10.1172/JC11753).

Preferably, the ATP-hydrolyzing enzyme is soluble (secreted), i.e. not bound or attached to a (plasma) membrane. Without being bound to any theory, the present inventors assume that soluble ATP-hydrolyzing enzymes can reach various places (e.g., in the body) more efficiently as compared to membrane-bound enzymes. In particular, without being bound to any theory, it is assumed that the ATP-hydrolyzing enzyme mediates its beneficial effects (on dysbiosis or a dysbiosis-related disease) in the intestinal lumen, namely, by degrading extracellular ATP (eATP) released from microbiota in the gut. As membrane-bound ATP-hydrolyzing enzymes, such as endogenous CD39, cannot affect (the majority of) the extracellular ATP released by the microbiota in the gut, due to their limited activity range in the tissue where they are located, the ATP hydrolyzing enzyme is preferably not bound or attached to a (plasma) membrane. Accordingly, the ATP-hydrolyzing enzyme is preferably a soluble ATP-hydrolyzing enzyme.

Examples of soluble ATP-hydrolyzing enzymes include bacterial (e.g., Shigella spp., in particular Shigella flexneri, or Legionella spp., in particular Legionella pneumophila,), Toxoplasma gondii, Trypanosoma spp., and potato apyrase as well as (engineered) soluble CD39.

Preferably, the ATP-hydrolyzing enzyme is apyrase. Apyrases are ATP-diphosphohydrolases that catalyze the sequential hydrolysis of ATP to ADP and ADP to AMP releasing inorganic phosphate. In particular, apyrases can also act on ADP and other nucleoside triphosphates and diphosphates in addition to ATP. Apyrase can be found in various eukaryotes in membrane bound and/or secreted soluble forms.

In general, the apyrase may have the sequence of any naturally occurring apyrase from any organism. In some embodiments, the apyrase is not an endogenous apyrase. In other words, the apyrase differs from the endogenous apyrase of the organism, to which it is administered. In certain embodiments, the apyrase is not a human endogenous apyrase, e.g. the apyrase may be a non-human apyrase. In some embodiments, the apyrase is not a mammalian apyrase. Preferably, the apyrase may be a bacterial or plant apyrase. Non-limiting examples of potent ATP-hydrolyzing enzymes useful for the present invention include soluble CD39 and apyrase of Shigella spp., in particular Shigella flexneri, Legionella spp., in particular Legionella pneumophila, Toxoplasma gondii, Trypanosoma spp., and Solanum tuberosum (potato). For example, the apyrase may be Shigella flexneri apyrase or Solanum tuberosum (potato) apyrase. Moreover, the apyrase may be sequence variant of a naturally occurring apyrase exhibiting at least 50% or 60%, preferably at least 70% or 75%, more preferably at least 80% or 85%, even more preferably at least 90% or 95%, still more preferably at least 97% or 98%, such as at least 99% sequence identity to a naturally occurring apyrase. In particular, such a sequence variant may be functional, i.e., the ATP-hydrolyzing function of the apyrase is maintained in the sequence variant. The skilled person is aware of various bioinfomatics tools providing annotated sequences of proteins, including apyrases, and identifying active sites, domains and regions (such as nucleotide binding regions) important for the ATP-hydrolyzing functionality of a certain apyrase. Accordingly, the skilled person is well-aware, which amino acid positions must be maintained in an apyrase to maintain its ATP-hydrolyzing functionality. Preferably, the apyrase comprises the amino acid sequence of SEQ ID NO: 1. Also included are functional sequence variants of SEQ ID NO: 1 as described above, i.e. having at least 50% or 60%, preferably at least 70% or 75%, more preferably at least 80% or 85%, even more preferably at least 90% or 95%, still more preferably at least 97% or 98%, such as at least 99% sequence identity to a naturally occurring apyrase. In sequence variants of SEQ ID NO: 1 R192 must be maintained to ensure functionality.

The ATP-hydrolyzing enzyme may be obtained by any means. Preferably, the ATP-hydrolyzing enzyme is recombinantly produced. Preferably, the ATP-hydrolyzing enzyme is recombinantly produced apyrase. Preferably, the apyrase is recombinantly produced apyrase having the sequence of SEQ ID NO: 1 or a sequence variant thereof as described above, e.g. having at least 70% or 75%, more preferably at least 80% or 85%, even more preferably at least 90% or 95%, still more preferably at least 97% or 98%, such as at least 99% sequence identity; wherein R192 is preferably maintained. For recombinant production, the ATP-hydrolyzing enzyme may be encoded by a nucleic acid not naturally occurring in the cell or organism expressing the ATP-hydrolyzing enzyme. Recombinant production of the ATP-hydrolyzing enzyme may be achieved, for example, (1) by heterologous expression (wherein the apyrase sequence is derived from a different organism than the organism used for its expression), (2) by expression based on an expression vector (not occurring in nature; e.g. for overexpression of the ATP-hydrolyzing enzyme), (3) by not naturally occurring ATP-hydrolyzing enzymes (e.g., functional sequence variants as described above), or by any combination of (1)-(3). For example, a (heterologous) cell expressing the ATP-hydrolyzing enzyme may impart a post-translational modification (PTM; e.g., glycosylation) on the ATP-hydrolyzing enzyme that is not present in its native state. Such a PTM may result in a functional difference (e.g., reduced immunogenicity). Accordingly, the ATP-hydrolyzing enzyme may have a post-translational modification, which is distinct from the naturally produced ATP-hydrolyzing enzyme. As an alternative, the apyrase may be used directly from a natural source. The apyrase may be obtained from a plant source, an animal source or a bacterial source. The apyrase may be purified or cell extracts (such as periplasmic extracts of bacterial cells) may be used.

While the ATP-hydrolyzing enzyme may be used as protein/polypeptide, the ATP-hydrolyzing enzyme as described herein may also be encoded by a polynucleotide comprised in a nucleic acid. Accordingly, the present invention also provides a nucleic acid molecule comprising a polynucleotide encoding the ATP-hydrolyzing enzyme as described herein for use in the treatment of dysbiosis. A nucleic acid (molecule) is a molecule comprising nucleic acid components. The term nucleic acid molecule usually refers to DNA or RNA molecules. It may be used synonymous with the term “polynucleotide”, i.e. the nucleic acid molecule may consist of a polynucleotide encoding the ATP-hydrolyzing enzyme. Alternatively, the nucleic acid molecule may also comprise further elements in addition to the polynucleotide encoding the ATP-hydrolyzing enzyme. Typically, a nucleic acid molecule is a polymer comprising or consisting of nucleotide monomers which are covalently linked to each other by phosphodiester-bonds of a sugar/phosphate-backbone. The term “nucleic acid molecule” also encompasses modified nucleic acid molecules, such as base-modified, sugar-modified or backbone-modified etc. DNA or RNA molecules. Examples of nucleic acid molecules and/or polynucleotides include, e.g., a recombinant polynucleotide, a vector, an oligonucleotide, an RNA molecule such as an rRNA, an mRNA, an miRNA, an siRNA, or a tRNA, or a DNA molecule such as a cDNA.

Due to the redundancy of the genetic code, the present invention also comprises sequence variants of nucleic acid sequences, which encode the same amino acid sequences. For example, the polynucleotide encoding the apyrase having the amino acid sequence of SEQ ID NO: 1 may have the nucleotide sequence of SEQ ID NO: 3 or a sequence variant thereof encoding the same amino acid sequence of SEQ ID NO: 1 (due to the redundancy of the genetic code).

The polynucleotide encoding the ATP-hydrolyzing enzyme (or the complete nucleic acid molecule) may be optimized for expression of the ATP-hydrolyzing enzyme. For example, codon optimization of the nucleotide sequence may be used to improve the efficiency of translation in expression systems for the production of the ATP-hydrolyzing enzyme. Accordingly, the polynucleotide encoding of the ATP-hydrolyzing enzyme may be codon-optimized. The skilled artisan is aware of various tools for codon optimization, such as those described in: Ju Xin Chin, Bevan Kai-Sheng Chung, Dong-Yup Lee, Codon Optimization OnLine (COOL): a web-based multi-objective optimization platform for synthetic gene design, Bioinformatics, Volume 30, Issue 15, 1 Aug. 2014, Pages 2210-2212; or in: Grote A, Hiller K, Scheer M, Munch R, Nortemann B, Hempel D C, Jahn D, JCat: a novel tool to adapt codon usage of a target gene to its potential expression host. Nucleic Acids Res. 2005 Jul. 1; 33(Web Server issue):W526-31; or, for example, Genscript's OptimumGene™ algorithm (as described in US 2011/0081708 A1).

Moreover, the nucleic acid molecule may comprise heterologous elements (i.e., elements, which in nature do not occur on the same nucleic acid molecule as the coding sequence for the ATP-hydrolyzing enzyme), e.g. for expression (such as heterologous expression) of the ATP-hydrolyzing enzyme. For example, a nucleic acid molecule may comprise a heterologous promotor, a heterologous enhancer, a heterologous UTR (e.g., for optimal translation/expression), a heterologous Poly-A-tail, and the like. In some embodiments the nucleic acid molecule may comprise an element conferring resistance against an antibiotic. In other embodiments, the nucleic acid molecule does not comprise an element conferring resistance against an antibiotic.

In general, the nucleic acid molecule may be manipulated to insert, delete or alter certain nucleic acid sequences. Changes from such manipulation include, but are not limited to, changes to introduce restriction sites, to amend codon usage, to add or optimize transcription and/or translation regulatory sequences, etc. It is also possible to change the nucleic acid to alter the encoded amino acids. For example, it may be useful to introduce one or more (e.g., 1, 2, 3, 4, 5, 6, 7, 8, 9, 10, etc.) amino acid substitutions, deletions and/or insertions into the amino acid sequence of the ATP-hydrolyzing enzyme. Such point mutations can modify stability, post-translational modifications, expression yield, etc.; can introduce amino acids for the attachment of covalent groups (e.g., labels); or can introduce tags (e.g., for purification purposes). Alternatively, a mutation in a nucleic acid sequence may be “silent”, i.e. not reflected in the amino acid sequence due to the redundancy of the genetic code as described above. In general, mutations can be introduced in specific sites or can be introduced at random, followed by selection (e.g., molecular evolution). For instance, a nucleic acid encoding the ATP-hydrolyzing enzyme can be randomly or directionally mutated to introduce different properties in the encoded amino acids. Such changes can be the result of an iterative process wherein initial changes are retained and new changes at other nucleotide positions are introduced. Further, changes achieved in independent steps may be combined.

In some embodiments, the nucleic acid molecule comprising a polynucleotide encoding the ATP-hydrolyzing enzyme may be a vector, for example an expression vector. A vector is usually a recombinant nucleic acid molecule, i.e. a nucleic acid molecule which does not occur in nature. Accordingly, the vector may comprise heterologous elements (i.e., sequence elements of different origin in nature). For example, the vector may comprise a multi cloning site, a heterologous promotor, a heterologous enhancer, a heterologous selection marker (to identify cells comprising said vector in comparison to cells not comprising said vector) and the like. A vector in the context of the present invention is suitable for incorporating or harboring a desired nucleic acid sequence. Such vectors may be storage vectors, expression vectors, cloning vectors, transfer vectors etc. A storage vector is a vector which allows the convenient storage of a nucleic acid molecule. Thus, the vector may comprise a sequence corresponding, e.g., to the ATP-hydrolyzing enzyme. An expression vector may be used for production of expression products such as RNA, e.g. mRNA, or peptides, polypeptides or proteins. For example, an expression vector may comprise sequences needed for transcription of a sequence stretch of the vector, such as a (heterologous) promoter sequence. A cloning vector is typically a vector that contains a cloning site, which may be used to incorporate nucleic acid sequences into the vector. A cloning vector may be, e.g., a plasmid vector or a bacteriophage vector. A transfer vector may be a vector which is suitable for transferring nucleic acid molecules into cells or organisms, for example, viral vectors. A vector in the context of the present invention may be, e.g., an RNA vector or a DNA vector. For example, a vector in the sense of the present application may comprise a cloning site, a selection marker, and a sequence suitable for multiplication of the vector, such as an origin of replication. A vector in the context of the present application may be a plasmid vector.

In some embodiments, the vector is an expression vector. Expression vectors may be capable of enhancing the expression of one or more polynucleotides that have been inserted or cloned into the vector. Examples of such expression vectors include, bacteriophages, autonomously replicating sequences (ARS), centromeres, and other sequences which are able to replicate or be replicated in vitro or in a cell, or to convey a nucleic acid segment to a particular location within a cell of an animal or human. Expression vectors useful in the present invention include chromosomal-, episomal- and virus-derived vectors, e.g., vectors derived from bacterial plasmids or bacteriophages, and vectors derived from combinations thereof, such as cosmids and phagemids or virus-based vectors such as adenovirus, AAV, lentiviruses.

The expression vector may be a plasmid. Any plasmid expression vector may be used provided that it is replicable and viable in the host.

For expression of the ATP-hydrolyzing enzyme in bacteria, the expression vector is preferably a vector optimized for protein expression in bacteria, e.g. in E. coli. Such expression vectors are well-known in the art and commercially available. For example, the pBAD vector system may be used, which provides a reliable and controllable system for expressing recombinant proteins in bacteria. This system is based on the araBAD operon, which controls E. coli L-arabinose metabolism. The polynucleotide encoding the ATP-hydrolyzing enzyme may be placed into the pBAD vector downstream of the araBAD promoter, which then drives expression of the ATP-hydrolyzing enzyme in response to L-arabinose, and is inhibited by glucose.

In some embodiments, the expression vector may be mini-circle DNA. Mini-circle DNA are useful for persistently high levels of nucleic acid transcription. The circular vectors are characterized by being devoid of expression-silencing bacterial sequences. For example, mini-circle vectors differ from bacterial plasmid vectors in that they lack an origin of replication, and lack drug selection markers commonly found in bacterial plasmids, e.g. β-lactamase, tet, and the like. Consequently, minicircle DNA becomes smaller in size, allowing more efficient delivery.

In certain embodiments, the expression vector may be a viral vector. Any viral vector based on any virus may be used as a carrier for the agent. Commonly used classes of viral systems used in gene therapy can be categorized into two groups according to whether their genomes integrate into host cellular chromatin (oncoretroviruses and lentiviruses) or persist in the cell nucleus predominantly as extrachromosomal episomes (adeno-associated viruses, adenoviruses and herpesviruses). Accordingly, the viral vector may be a retroviral, lentiviral, adenoviral, herpesviral or adeno-associated viral vector, as described below. Moreover, the viral vector may be derived from any of retroviruses, lentiviruses, adeno-associated viruses, adenoviruses or herpesviruses.

The viral vector may be an adenoviral (AdV) vector. Adenoviruses are medium-sized double-stranded, non-enveloped DNA viruses with linear genomes that is between 26-48 Kbp. Adenoviruses gain entry to a target cell by receptor-mediated binding and internalization, penetrating the nucleus in both non-dividing and dividing cells. Adenoviruses are heavily reliant on the host cell for survival and replication and are able to replicate in the nucleus of vertebrate cells using the host's replication machinery.

The viral vector may be from the Parvoviridae family. The Parvoviridae is a family of small single-stranded, non-enveloped DNA viruses with genomes approximately 5000 nucleotides long. The viral vector may be an adeno-associated virus (AAV). AAV is a dependent parvovirus that generally requires co-infection with another virus (typically an adenovirus or herpesvirus) to initiate and sustain a productive infectious cycle. In the absence of such a helper virus, AAV is still competent to infect or transduce a target cell by receptor-mediated binding and internalization, penetrating the nucleus in both non-dividing and dividing cells. Because progeny virus is not produced from AAV infection in the absence of helper virus, the extent of transduction is restricted only to the initial cells that are infected with the virus. Unlike retrovirus, adenovirus, and herpes simplex virus, AAV appears to lack human pathogenicity and toxicity.

Viral vectors based on viruses from the family Retroviridae may be used. Retroviruses comprise single-stranded RNA animal viruses that are characterized by two unique features. First, the genome of a retrovirus is diploid, consisting of two copies of the RNA. Second, this RNA is transcribed by the virion-associated enzyme reverse transcriptase into double-stranded DNA. This double-stranded DNA or provirus can then integrate into the host genome and be passed from parent cell to progeny cells as a stably-integrated component of the host genome.

Preferably, the expression vector is a plasmid. As an alternative, preferably the expression vector is a bacteriophage. Where the expression vector is a plasmid or a bacteriophage, the expression vector may be transformed into a bacterial cell and the bacterial cell included in the composition of the invention. The bacterial cell may be E. coli. As an alternative the bacterial carrier may be attenuated Salmonella enterica. The attenuated Salmonella enterica may be of the serovar Salmonella Typhimurium.

In some embodiments, the nucleic acid molecule comprising a polynucleotide encoding the ATP-hydrolyzing enzyme as described herein may be a genomic nucleic acid molecule, for example genomic DNA (e.g. chromosomal DNA). In other words, the polynucleotide encoding the ATP-hydrolyzing enzyme may be integrated into the genome (of an organism) (heterologously) expressing the ATP-hydrolyzing enzyme.

In some embodiments, a DNA fragment may be introduced into, e.g., a host cell/microorganism, such as a bacterium, for integration into the genome of the host cell/microorganism, such as a bacterium. To this end, the DNA fragment may contain a nucleotide sequence encoding the ATP-hydrolyzing enzyme, in particular an apyrase, as described herein (for example the S. flexneri phoN2 gene or a sequence variant thereof) for the integration into the genome, e.g. of a host cell/microorganism, such as a bacterium. For example, such a DNA fragment may be for the integration of S. flexneri phoN2 gene in E. coli Nissle (EcN) genome. An exemplified DNA fragment for the integration of S. flexneri phoN2 gene in E. coli Nissle (EcN) genome is shown in FIG. 52. In some embodiments, the DNA fragment may contain malP: EcN gene for maltodextrin phosphorylase; cat: E. coli gene for chloramphenicol acetyltransferase; phoN2: S. flexneri gene for apyrase; malT: EcN gene for the transcriptional activator of the maltose and maltodextrins operon; FRT: Flippase Recognition Target sequence; P_{cot}: promoter of the cat gene; P_{proD}: promoter of the phoN2 gene; BBa_BB0032 RBS: Ribosome Binding Site of the phoN2 gene; and/or TphoN2: transcriptional terminator of the phoN2 gene. In some embodiments, the nucleotide sequence of the EcN malP gene portion is according to SEQ ID NO: 6 or a sequence variant thereof having at least 75%, 80%, 85%, 90% or 95% sequence identity. In some embodiments, the nucleotide sequence of the EcN malT gene portion is according to SEQ ID NO: 7 or a sequence variant thereof having at least 75%, 80%, 85%, 90% or 95% sequence identity. In some embodiments, the DNA fragment including the P_{proD} promoter, the BBa_BB0032 RBS, the S. flexneri phoN2 gene and the phoN2 transcriptional terminator may be according to SEQ ID NO: 8 or a sequence variant thereof having at least 75%, 80%, 85%, 90% or 95% sequence identity. In some embodiments, the DNA fragment including the E. coli cat gene flanked by the FRT sequences may be according to SEQ ID NO: 9 or a sequence variant thereof having at least 75%, 80%, 85%, 90% or 95% sequence identity.

Host Cells, Microorganisms and Viral Particles

In a further aspect, the present invention also provides a host cell comprising the nucleic acid molecule as described herein, i.e. the nucleic acid comprising the polynucleotide encoding the ATP-hydrolyzing enzyme as described herein, for use in the treatment of dysbiosis.

Host cells may be prokaryotic or eukaryotic cells. Examples of such cells include but are not limited to, eukaryotic cells, e.g., yeast cells, animal cells or plant cells or prokaryotic cells, including E. coli. In some embodiments, the cells may be mammalian cells, such as a mammalian cell line. Examples include human cells, CHO cells, HEK293T cells, PER.C6 cells, NSO cells, human liver cells, or myeloma cells.

The cell may be transformed or transfected with a nucleic acid, such as a (expression) vector, as described above. The term “transfection” refers to the introduction of nucleic acid molecules, such as DNA or RNA molecules (e.g. plasmids), into eukaryotic animal/human cells, while the term “transformation” usually refers to the introduction of nucleic acid molecules, such as DNA or RNA molecules (e.g. plasmids), into bacterial cells, yeast cells, plant cells or fungi cells. In the context of the present invention, the terms “transfection” and “transformation” encompass any method known to the skilled person for introducing nucleic acid molecules into cells, such as into mammalian or bacterial cells. Such methods encompass, for example, electroporation, lipofection, e.g. based on cationic lipids and/or liposomes, calcium phosphate precipitation, nanoparticle based transfection, virus based transfection, or transfection based on cationic polymers, such as DEAE-dextran or polyethylenimine etc. In some embodiments, the introduction is non-viral. For bacterial cells, competent bacteria may be used for transformation.

Moreover, the cells of the present invention may be transfected/transformed stably or transiently with the nucleic acid (vector), e.g. for expressing the ATP-hydrolyzing enzyme as described herein. In some embodiments, the cells are stably transfected with the nucleic acid (vector) comprising a polynucleotide encoding the ATP-hydrolyzing enzyme as described herein. In other embodiments, the cells are transiently transfected/transformed with the nucleic acid (vector) comprising a polynucleotide encoding the ATP-hydrolyzing enzyme as described herein.

Accordingly, the present invention also provides a recombinant host cell, which heterologously expresses the ATP-hydrolyzing enzyme as described herein for use in the treatment of dysbiosis. For example, the cell may be of another species than the ATP-hydrolyzing enzyme. In some embodiments, the cell type of the cell does not express (such) an ATP-hydrolyzing enzyme in nature. Moreover, the host cell may impart a post-translational modification (PTM; e.g., glycosylation) on the ATP-hydrolyzing enzyme that is not present in their native state. Such a PTM may result in a functional difference (e.g., reduced immunogenicity). Accordingly, the ATP-hydrolyzing enzyme may have a post-translational modification, which is distinct from the naturally produced ATP-hydrolyzing enzyme.

In a further aspect, the present invention also provides a microorganism comprising the nucleic acid molecule as described herein, i.e. the nucleic acid comprising the polynucleotide encoding the ATP-hydrolyzing enzyme as described herein, for use in the treatment of dysbiosis. The microorganism may be a recombinant microorganism, which heterologously expresses the ATP-hydrolyzing enzyme as described herein. For example, the microorganism may be of another species than the ATP-hydrolyzing enzyme. In some embodiments, the microorganism may be a recombinant microorganism overexpressing the ATP-hydrolyzing enzyme as described herein. The microorganism may be a live microorganism.

As used herein, the term “microorganism” refers to a microscopic organism, which may exist in its single-celled form or in a colony of cells. Typically, the term “microorganism” includes all unicellular organisms. Accordingly, the microorganism may be selected from prokaryotes, such as archea and bacteria, and eukaryotes, such as unicellular protists, protozoans, fungi and plants.

Preferably, the microorganism is a prokaryotic microorganism, such as a bacterium, or a eukaryotic microorganism, such as a yeast. In some embodiments, the microorganism is selected from the group consisting of Escherichia spp., Salmonella spp., Yersinia spp., Vibrio spp., Listeria spp., Lactococcus spp., Shigella spp., Cyanobacteria, and Saccharomyces spp. As used herein, the expression “spp.” in connection with any microorganism is intended to comprise all members of a given genus, including species, subspecies and others.

In certain embodiments, the microorganisms may be provided as probiotics (e.g., of live bacteria). As used herein, the term “probiotics” refers to live microorganisms, such as bacteria or yeasts, providing health benefits when consumed, for example by improving or restoring the gut flora. Such live microorganisms can be used as food additive due to the health benefits they can provide. Those can be for example lyophilized in granules, pills or capsules, or directly mixed with dairy products for consumption. Examples of microorganisms, for which health benefits have been demonstrated include, but are not limited to Lactobacillus, Bifidobacterium, Saccharomyces, Lactococcus, Enterococcus, Streptococcus, Pediococcus, Leuconostoc, Bacillus, Escherichia coli, in particular regarding probiotic strains thereof, such as those described in Fijan S. Microorganisms with claimed probiotic properties: an overview of recent literature. IntJ Environ Res Public Health. 2014; 11(5):4745-4767. doi:10.3390/ijerph110504745, which is incorporated herein by reference.

In case of virulent microorganisms, the virulence of the microorganism may be attenuated. Methods for attenuating the virulence, e.g. of bacteria, are known in the art and described, for example, in WO 2018/089841. In general, attenuation of virulence may be achieved by a mutation of a virulence factor from a virulent pathogen.

In particular, the present invention provides a bacterium (bacterial cell) comprising the nucleic acid molecule as described herein, i.e. the nucleic acid comprising the polynucleotide encoding the ATP-hydrolyzing enzyme as described herein, for use in the treatment of dysbiosis. Accordingly, the host cell as described above may be a bacterial cell and the microorganism as described above may be a bacterium.

The bacterium may be a recombinant bacterium, i.e. a bacterium, which does not occur in nature. In particular, the recombinant bacterium may comprise nucleic acid sequences not occurring in the bacterium in nature, e.g. for heterologous expression or overexpression of the ATP-hydrolyzing enzyme. Accordingly, the bacterium may heterologously express the ATP-hydrolyzing enzyme (i.e., the expressed ATP-hydrolyzing enzyme may not naturally occur in the bacterium and may be derived from a distinct strain, species etc.); or the bacterium may overexpress the ATP-hydrolyzing enzyme. As used herein, the term “overexpression” refers to artificial expression of a gene of interest (e.g. encoding the ATP-hydrolyzing enzyme) in increased quantity. Overexpression can be achieved by various ways, e.g. by increasing the number of nucleic acid molecules encoding the gene of interest (e.g. encoding the ATP-hydrolyzing enzyme) and/or by the use of regulatory elements increasing expression (e.g. promoters, enhancers or other gene-regulatory elements).

The bacterium may be a live bacterium. If the bacterium is a pathogen, its virulence may be attenuated as described above. In general, the bacterium may be selected from Gram-positive or Gram-negative bacteria. In some embodiments, the bacterium may be a Gram-negative bacterium, such as a bacterium selected from Escherichia spp., Salmonella spp., Yersinia spp., Vibrio spp., Shigella spp., or Cyanobacteria, such as a bacterium selected from Escherichia coli, Salmonella typhi, Salmonella typhimurium, Yersinia enterocolitica, Vibrio cholerae, and Shigella flexneri. In some embodiments, the bacterium may be a Gram-positive bacterium. Examples of Gram-positive bacteria include Lactococcus spp., such as Lactococcus lactis, and Listeria spp., such as Listeria monocytogenes. Preferably, the bacterium may be Escherichia coli, Lactococcus lactis or Salmonella typhimurium. Particularly preferably, the bacterium may be Escherichia coli, Lactococcus lactis or Salmonella typhimurium, in particular (heterologously) expressing apyrase.

The bacterium may provide probiotic properties, as described above. In particular, the probiotic bacterium may be Lactococcus lactis or a probiotic strain of Escherichia coli, such as Escherichia coli Nissle 1917 (EcN). Escherichia coli Nissle 1917 was shown to treat constipation (Chmielewska A., Szajewska H. Systematic review of randomised controlled trials: Probiotics for functional constipation. World J. Gastroenterol. 2010; 16:69-75) and inflammatory bowel disease (Behnsen J., Deriu E., Sassone-Corsi M., Raffatellu M. Probiotics: Properties, examples, and specific applications. Cold Spring Harb. Perspect. Med. 2013; 3 doi: 10.1101/cshperspect.a010074) and to relieve gastrointestinal disorder, ulcerative colitis, and Crohn's disease (Xia P., Zhu J., Zhu G. Escherichia coli Nissle 1917 as safe vehicles for intestinal immune targeted therapy-A review. Acta Microbiol. Sin. 2013; 53:538-544).

In a further aspect, the present invention also provides a viral particle comprising the nucleic acid molecule as described herein, i.e. the nucleic acid comprising the polynucleotide encoding the ATP-hydrolyzing enzyme as described herein, for use in the treatment of dysbiosis. The viral particle may be a recombinant microorganism, e.g. heterologously expressing the ATP-hydrolyzing enzyme as described herein. As used herein, the term “viral particle” includes virions as well as virus-like particles. A “virion” (“virus”) is a structure, which can usually transfer nucleic acid from one cell to another, and may be “enveloped” or “non-enveloped”.

As used herein, a “virus-like particle” (also “VLP”) refers in particular to a non-replicating, viral shell, derived from any of several viruses. VLPs lack the viral components that are required for virus replication and thus represent a highly attenuated form of a virus. VLPs are generally composed of one or more viral proteins, such as, but not limited to, those proteins referred to as capsid, coat, shell, surface and/or envelope proteins, or particle-forming polypeptides derived from these proteins. VLPs can form spontaneously upon recombinant expression of the protein in an appropriate expression system. Virus like particles and methods of their production are known and familiar to the person of ordinary skill in the art, and viral proteins from several viruses are known to form VLPs, including human papillomavirus, HIV (Kang et al., Biol. Chem. 380: 353-64 (1999)), Semliki-Forest virus (Notka et al., Biol. Chem. 380: 341-52 (1999)), human polyomavirus (Goldmann et al., J. Virol. 73: 4465-9 (1999)), rota virus (Jiang et al., Vaccine 17: 1005-13 (1999)), parvovirus (Casal, Biotechnology and Applied Biochemistry, Vol 29, Part 2, pp 141-150 (1999)), canine parvovirus (Hurtado et al., J. Viral. 70: 5422-9 (1996)), hepatitis E virus (Li et al., J. Viral. 71:35 7207-13 (1997)), and Newcastle disease virus. The formation of such VLPs can be detected by any suitable technique. Examples of suitable techniques known in the art for detection of VLPs in a medium include, e.g., electron microscopy techniques, dynamic light scattering (DLS), selective chromatographic separation (e.g., ion exchange, hydrophobic interaction, and/or size exclusion chromatographic separation of the VLPs) and density gradient centrifugation. Further, VLPs can be isolated by known techniques, e.g., density gradient centrifugation and identified by characteristic density banding. See, for example, Baker et al. (1991) Biophys. J. 60: 1445-1456; and Hagensee et al. (1994) J. Viral. 68:4503-4505; Vincente, J Invertebr Pathol., 2011; Schneider-Ohrum and Ross, Curr. Top. Microbial. Immunol., 354: 53073 (2012).

Preferably, the viral particle is not infectious in humans. In particular, viruses infecting and replicating in bacteria, such as bacteriophages, may be used. Accordingly, the present invention also provides a bacteriophage comprising the nucleic acid molecule as described herein, i.e. the nucleic acid comprising the polynucleotide encoding the ATP-hydrolyzing enzyme as described herein, for use in the treatment of dysbiosis. A bacteriophage is a virus that infects and replicates within bacteria and archaea. Bacteriophages are usually composed of proteins that encapsulate a DNA or RNA genome, and occur in various distinct structures, that may be either simple or elaborate. Phages may provide antibacterial effects. Bacteriophages comprising the nucleic acid comprising the polynucleotide encoding the ATP-hydrolyzing enzyme may readily transfer the nucleic acid comprising the polynucleotide encoding the ATP-hydrolyzing enzyme to bacteria, such that the ATP-hydrolyzing enzyme is expressed by bacteria.

Compositions

Each of the ATP hydrolyzing enzyme, the nucleic acid comprising the polynucleotide encoding the ATP hydrolyzing enzyme, the host cell comprising the nucleic acid comprising the polynucleotide encoding the ATP hydrolyzing enzyme, the microorganism comprising the nucleic acid comprising the polynucleotide encoding the ATP hydrolyzing enzyme, and the viral particle comprising the nucleic acid comprising the polynucleotide encoding the ATP hydrolyzing enzyme may be provided in a composition. The composition may be a vaccine. Accordingly, the present invention also provides a (pharmaceutical) composition comprising any one of the ATP hydrolyzing enzyme, the nucleic acid comprising the polynucleotide encoding the ATP hydrolyzing enzyme, the host cell comprising the nucleic acid comprising the polynucleotide encoding the ATP hydrolyzing enzyme, the microorganism comprising the nucleic acid comprising the polynucleotide encoding the ATP hydrolyzing enzyme, and the viral particle comprising the nucleic acid comprising the polynucleotide encoding the ATP hydrolyzing enzyme for use in the treatment of dysbiosis or a dysbiosis-related disease.

For example, the composition may be a pharmaceutical composition, which may optionally comprise a pharmaceutically acceptable carrier, diluent and/or excipient. Although the carrier, diluent or excipient may facilitate administration, it should not itself be harmful to the individual receiving the composition. Nor should it be toxic. Usually, carriers, diluents and excipients are not “active” components of the composition. Accordingly, the ATP hydrolyzing enzyme, the host cell comprising the nucleic acid comprising the polynucleotide encoding the ATP hydrolyzing enzyme, the microorganism comprising the nucleic acid comprising the polynucleotide encoding the ATP hydrolyzing enzyme, or the viral particle comprising the nucleic acid comprising the polynucleotide encoding the ATP hydrolyzing enzyme may be the sole active component of the composition (i.e. which is pharmaceutically active, in particular with regard to the disease to be treated). Suitable carriers may be large, slowly metabolized macromolecules such as proteins, polypeptides, liposomes, polysaccharides, polylactic acids, polyglycolic acids, polymeric amino acids, amino acid copolymers and inactive virus particles.

Pharmaceutically acceptable salts can be used, for example mineral acid salts, such as hydrochlorides, hydrobromides, phosphates and sulphates, or salts of organic acids, such as acetates, propionates, malonates and benzoates.

The composition may comprise a vehicle. A vehicle is typically understood to be a material that is suitable for storing, transporting, and/or administering a compound, such as a pharmaceutically active compound. For example, the vehicle may be a physiologically acceptable liquid, which is suitable for storing, transporting, and/or administering a pharmaceutically active compound. Once formulated, the compositions can be administered directly to the subject. In some embodiments the compositions are adapted for administration to mammalian, e.g., human subjects.

In some embodiments, the pharmaceutical composition may include an antimicrobial, particularly if packaged in a multiple dose format. They may comprise detergent e.g., a Tween (polysorbate), such as Tween 80. Detergents are generally present at low levels e.g., less than 0.01%. Compositions may also include sodium salts (e.g., sodium chloride) to give tonicity. For example, a concentration of 10±2 mg/ml NaCl is typical.

Further, pharmaceutical compositions may comprise a sugar alcohol (e.g., mannitol) or a disaccharide (e.g., sucrose or trehalose) e.g., at around 15-30 mg/ml (e.g., 25 mg/ml), particularly if they are to be lyophilized or if they include material which has been reconstituted from lyophilized material. The pH of a composition for lyophilization may be adjusted to between 5 and 8, or between 5.5 and 7, or around 6.1 prior to lyophilization.

Pharmaceutically acceptable carriers in a pharmaceutical composition may additionally contain liquids such as water, saline, glycerol and ethanol. Additionally, auxiliary substances, such as wetting or emulsifying agents or pH buffering substances, may be present in such compositions. Such carriers enable the pharmaceutical compositions to be formulated as tablets, pills, dragees, capsules, liquids, gels, syrups, slurries and suspensions, for ingestion by the subject. A thorough discussion of pharmaceutically acceptable carriers is available in Gennaro (2000) Remington: The Science and Practice of Pharmacy, 20th edition, ISBN: 0683306472.

Pharmaceutical compositions may be prepared in various forms and may be administered by any number of routes including, but not limited to, oral, intravenous, intramuscular, intra-arterial, intraperitoneal, subcutaneous, enteral, sublingual, or rectal routes. Preferably, the pharmaceutical composition may be prepared for oral administration, e.g. as tablets, capsules and the like, or as injectable, e.g. as liquid solutions or suspensions. In some embodiments, the pharmaceutical composition is an injectable. Solid forms suitable for solution in, or suspension in, liquid vehicles prior to injection are also encompassed, for example the pharmaceutical composition may be in lyophilized form.

The composition may be prepared for oral administration e.g., as a tablet or capsule, as a spray, or as a syrup (optionally flavored). Orally acceptable dosage forms include, but are not limited to, capsules, tablets, aqueous suspensions or solutions. In the case of tablets for oral use, carriers commonly used may include lactose and corn starch. Lubricating agents, such as magnesium stearate, may also be added. For oral administration in a capsule form, useful diluents include lactose and dried cornstarch. When aqueous suspensions are required for oral use, the active ingredient, i.e. the ATP hydrolyzing enzyme, the nucleic acid comprising the polynucleotide encoding the ATP hydrolyzing enzyme, the host cell comprising the nucleic acid comprising the polynucleotide encoding the ATP hydrolyzing enzyme, the microorganism comprising the nucleic acid comprising the polynucleotide encoding the ATP hydrolyzing enzyme, or the viral particle comprising the nucleic acid comprising the polynucleotide encoding the ATP hydrolyzing enzyme, may be combined with emulsifying and suspending agents. If desired, certain sweetening, flavoring or coloring agents may also be added. As such, the active component may be susceptible to degradation in the gastrointestinal tract. Thus, if the composition is to be administered by a route using the gastrointestinal tract, the composition may contain agents which protect the ATP-hydrolyzing enzyme from degradation but which release the ATP-hydrolyzing enzyme once it has been absorbed from the gastrointestinal tract. The composition may be in kit form, designed such that a combined composition is reconstituted just prior to administration to a subject. For example, a lyophilized ATP-hydrolyzing enzyme may be provided in kit form with sterile water or a sterile buffer.

Within the scope of the invention are compositions present in several forms adapted for various routes of administration; the forms include, but are not limited to, those forms suitable for parenteral administration, e.g., by injection or infusion, for example by bolus injection or continuous infusion. Where the product is for injection or infusion, it may take the form of a suspension, solution or emulsion in an oily or aqueous vehicle and it may contain formulatory agents, such as suspending, preservative, stabilizing and/or dispersing agents. Alternatively, the ATP-hydrolyzing enzyme may be in dry form, for reconstitution before use with an appropriate sterile liquid. In some embodiments, the compositions may be prepared as injectables, either as liquid solutions or suspensions. Solid forms suitable for solution in, or suspension in, liquid vehicles prior to injection can also be prepared (e.g., a lyophilized composition, e.g. for reconstitution with sterile water containing a preservative). For injection, e.g. intravenous, cutaneous or subcutaneous injection, or injection at the site of affliction, the active ingredient may be in the form of a parenterally acceptable aqueous solution which is pyrogen-free and has suitable pH, isotonicity and stability. Those of relevant skill in the art are well able to prepare suitable solutions using, for example, isotonic vehicles such as sodium chloride injection, Ringer's injection, lactated Ringer's injection. Preservatives, stabilizers, buffers, antioxidants and/or other additives may be included, as required. For injection, the pharmaceutical composition may be provided, for example, in a pre-filled syringe.

Pharmaceutical compositions may generally have a pH between 5.5 and 8.5, in some embodiments this may be between 6 and 8, for example about 7. The pH may be maintained by the use of a buffer. The composition may be sterile and/or pyrogen free. The composition may be gluten free. The composition may be isotonic with respect to humans. In some embodiments pharmaceutical compositions may be supplied in hermetically-sealed containers.

Whether it is a protein, a peptide, a nucleic acid molecule, a host cell, a microorganism, a viral particle or another pharmaceutically useful compound as described above that is to be given to an individual, administration is usually in an effective amount, e.g. a “prophylactically effective amount” or in a “therapeutically effective amount” (as the case may be), this being sufficient to show benefit to the individual. The actual amount administered, and rate and time-course of administration, will depend on the nature and severity of what is being treated. Accordingly, an “effective” amount of one or more active ingredients is usually an amount that is sufficient to treat, ameliorate, attenuate, reduce or prevent a desired disease or condition, or to exhibit a detectable therapeutic effect. Therapeutic effects also include reduction or attenuation in pathogenic potency or physical symptoms. The precise effective amount for any particular subject will depend upon their size, weight, and health, the nature and extent of the condition, and the therapeutics or combination of therapeutics selected for administration. The effective amount for a given situation is determined by routine experimentation and is within the judgment of a clinician.

The ATP hydrolyzing enzyme, the nucleic acid comprising the polynucleotide encoding the ATP hydrolyzing enzyme, the host cell comprising the nucleic acid comprising the polynucleotide encoding the ATP hydrolyzing enzyme, the microorganism comprising the nucleic acid comprising the polynucleotide encoding the ATP hydrolyzing enzyme, or the viral particle comprising the nucleic acid comprising the polynucleotide encoding the ATP hydrolyzing enzyme can be present either in the same pharmaceutical composition as the additional active component or, alternatively, comprised in a separate pharmaceutical composition. Accordingly, each additional active component may be comprised in a distinct pharmaceutical composition. Such different pharmaceutical compositions may be administered either combined/simultaneously or at separate times or at separate locations (e.g., separate parts of the body).

In certain embodiments, the ATP hydrolyzing enzyme may make up at least 50% by weight (e.g., 60%, 70%, 75%, 80%, 85%, 90%, 95%, 96%, 97%, 98%, 99% or more) of the total protein in the composition.

In some embodiments, the composition may contain the ATP hydrolyzing enzyme, the nucleic acid comprising the polynucleotide encoding the ATP hydrolyzing enzyme, the host cell comprising the nucleic acid comprising the polynucleotide encoding the ATP hydrolyzing enzyme, the microorganism comprising the nucleic acid comprising the polynucleotide encoding the ATP hydrolyzing enzyme, or the viral particle comprising the nucleic acid comprising the polynucleotide encoding the ATP hydrolyzing enzyme in purified form.

In some instances, the composition may contain a cell extract comprising the ATP hydrolyzing enzyme or the nucleic acid comprising the polynucleotide encoding the ATP hydrolyzing enzyme. For example, the composition may comprise a cell extract from a cell expressing the ATP hydrolyzing enzyme or a cell comprising the nucleic acid comprising the polynucleotide encoding the ATP hydrolyzing enzyme. Such a cell may be a bacterial cell as described above. For example, the composition may comprise a periplasmic extract of a bacterium comprising the nucleic acid comprising the polynucleotide encoding the ATP hydrolyzing enzyme. Preferred bacteria (bacterial cells) in this context are those described above.

In some embodiments, the composition may be formulated for administration in a nanocapsule. Preferably, the composition comprising the ATP hydrolyzing enzyme, the nucleic acid comprising the polynucleotide encoding the ATP hydrolyzing enzyme, the host cell comprising the nucleic acid comprising the polynucleotide encoding the ATP hydrolyzing enzyme, the microorganism comprising the nucleic acid comprising the polynucleotide encoding the ATP hydrolyzing enzyme, or the viral particle comprising the nucleic acid comprising the polynucleotide encoding the ATP hydrolyzing enzyme; may be formulated for administration in a nanocapsule. Accordingly, the present invention also provides a nanocapsule comprising the composition as described herein. In particular, the present invention provides a nanocapsule comprising (a composition comprising) the ATP hydrolyzing enzyme, the nucleic acid comprising the polynucleotide encoding the ATP hydrolyzing enzyme, the host cell comprising the nucleic acid comprising the polynucleotide encoding the ATP hydrolyzing enzyme, the microorganism comprising the nucleic acid comprising the polynucleotide encoding the ATP hydrolyzing enzyme, or the viral particle comprising the nucleic acid comprising the polynucleotide encoding the ATP hydrolyzing enzyme.

A nanocapsule is usually made from a nontoxic polymer/lipid and can protect substances from adverse environment. Nanocapsules are usually vesicular systems made of a polymeric membrane which encapsulates an inner liquid core at the nanoscale. Encapsulation methods are known in the art and include nanoprecipitation, emulsion-diffusion and solvent-evaporation. In some embodiments, the nanocapsule may be for enteral, in particular oral, administration. Nanocapsules and methods for preparing nanocapsules are known in the art and described, for example, in Erdogar N, Akkin S, Bilensoy E. Nanocapsules for Drug Delivery: An Updated Review of the Last Decade. Recent Pat Drug Deliv Formul. 2018; 12(4):252-266. doi: 10.2174/1872211313666190123153711, which is incorporated herein in its entirety.

The present invention also provides a method of preparing a (pharmaceutical) composition comprising the steps of: (i) preparing the ATP hydrolyzing enzyme, the nucleic acid comprising the polynucleotide encoding the ATP hydrolyzing enzyme, the host cell comprising the nucleic acid comprising the polynucleotide encoding the ATP hydrolyzing enzyme, the microorganism comprising the nucleic acid comprising the polynucleotide encoding the ATP hydrolyzing enzyme, or the viral particle comprising the nucleic acid comprising the polynucleotide encoding the ATP hydrolyzing enzyme; and (ii) admixing it with one or more pharmaceutically acceptable carriers.

Treatment of Dysbiosis and Dysbiosis-Related Diseases

As described above, the present invention provides
(a) an ATP hydrolyzing enzyme,
(b) a nucleic acid comprising a polynucleotide encoding the ATP hydrolyzing enzyme,
(c) a host cell comprising the nucleic acid,
(d) a microorganism comprising the nucleic acid, or
(e) a viral particle comprising the nucleic acid

for use in the treatment of dysbiosis or a dysbiosis-related disease.

The ATP-hydrolyzing enzyme as described above, the nucleic acid as described above encoding the ATP-hydrolyzing enzyme, or the host cell as described above, the microorganism as described above or the viral particle as described above is able to counteract dysbiosis and to inhibit or decrease symptoms of dysbiosis and dysbiosis-related diseases, as shown in the appended examples.

Accordingly, the present invention also provides a method for reducing the risk of occurrence, treating, ameliorating, or reducing dysbiosis or a dysbiosis-related disease in a subject in need thereof, comprising administering to the subject
(a) an ATP hydrolyzing enzyme,
(b) a nucleic acid comprising a polynucleotide encoding the ATP hydrolyzing enzyme,
(c) a host cell comprising the nucleic acid,
(d) a microorganism comprising the nucleic acid, or
(e) a viral particle comprising the nucleic acid.

In addition, the present invention also provides a method for restoring the balance of intestinal microbiota in a subject in need thereof, comprising administering to the subject
(a) an ATP hydrolyzing enzyme,
(b) a nucleic acid comprising a polynucleotide encoding the ATP hydrolyzing enzyme,
(c) a host cell comprising the nucleic acid,
(d) a microorganism comprising the nucleic acid, or
(e) a viral particle comprising the nucleic acid.

Dysbiosis and dysbiosis-related diseases are described above.

The ATP-hydrolyzing enzyme, the nucleic acid encoding the ATP-hydrolyzing enzyme, or the host cell, microorganism or viral particle comprising the nucleic acid encoding the ATP-hydrolyzing enzyme may be administered once or repeatedly (in the same treatment cycle). Thus, the administration may be repeated at least two times. Accordingly, the ATP-hydrolyzing enzyme, the nucleic acid encoding the ATP-hydrolyzing enzyme, or the host cell, microorganism or viral particle comprising the nucleic acid encoding the ATP-hydrolyzing enzyme may be administered repeatedly or continuously. The ATP-hydrolyzing enzyme, the nucleic acid encoding the ATP-hydrolyzing enzyme, or the host cell, microorganism or viral particle comprising the nucleic acid encoding the ATP-hydrolyzing enzyme may be administered repeatedly or continuously for a period of at least 1, 2, 3, or 4 weeks; 2, 3, 4, 5, 6, 8, 10, or 12 months; or 2, 3, 4, or 5 years. For example, the ATP-hydrolyzing enzyme, the nucleic acid encoding the ATP-hydrolyzing enzyme, or the host cell, microorganism or viral particle comprising the nucleic acid encoding the ATP-hydrolyzing enzyme may be administered twice per day, once per day (e.g., daily), every two days, every three days, once per week, every two weeks, every three weeks, once per month or every two months.

In some embodiments, ATP-hydrolyzing enzyme, the nucleic acid encoding the ATP-hydrolyzing enzyme, or the host cell, microorganism or viral particle comprising the nucleic acid encoding the ATP-hydrolyzing enzyme may be administered daily, e.g. for 3, 4, 5, 6, 7, 8, 9, 10, 11, 12, 13, 14, 15, 16, 17, 18, 19, 20, 21 or more (consecutive) days. In other embodiments, the ATP-hydrolyzing enzyme, the nucleic acid encoding the ATP-hydrolyzing enzyme, or the host cell, microorganism or viral particle comprising the nucleic acid encoding the ATP-hydrolyzing enzyme may be administered once or twice a week, e.g. for two or three weeks.

The ATP hydrolyzing enzyme, the nucleic acid, the host cell, the microorganism or the viral particle can be administered by various routes of administration, for example, systemically or locally. Routes for systemic administration in general include, for example, enteral and parenteral routes, which include subcutaneous, intravenous, intramuscular, intraarterial, intradermal and intraperitoneal routes. Preferably, the ATP hydrolyzing enzyme, the nucleic acid, the host cell, the microorganism or the viral particle is administered via an enteral route of administration. Enteral routes of administration refer to administration via the gastrointestinal tract and includes, for example oral, sublingual, and rectal administration as well as administration via a gastric tube. Oral administration of the ATP hydrolyzing enzyme, the nucleic acid comprising the polynucleotide encoding the ATP hydrolyzing enzyme, the host cell comprising the nucleic acid comprising the polynucleotide encoding the ATP hydrolyzing enzyme, the microorganism comprising the nucleic acid comprising the polynucleotide encoding the ATP hydrolyzing enzyme, or the viral particle comprising the nucleic acid comprising the polynucleotide encoding the ATP hydrolyzing enzyme is preferred. Without being bound to any theory, it is assumed that the ATP-hydrolyzing enzyme mediates its beneficial effects in the intestinal lumen, namely, by degrading extracellular ATP released from microbiota in the gut. Because enteral administration of the ATP hydrolyzing enzyme, the nucleic acid, the host cell, the microorganism or the viral particle delivers the ATP hydrolyzing enzyme into the gastrointestinal tract (gut), this route of administration is preferred for the ATP hydrolyzing enzyme, the nucleic acid, the host cell, the microorganism or the viral particle.

For example, the (encoded) ATP hydrolyzing enzyme may be a soluble ATP hydrolyzing enzyme; and the ATP hydrolyzing enzyme, the nucleic acid, the host cell, the microorganism or the viral particle may be administered via an enteral route of administration.

In some embodiments, dysbiosis or a dysbiosis-related disease is treated therapeutically, e.g. after diagnosis or strong assumption that a subject has dysbiosis or a dysbiosis-related disease, for example after detection of symptoms thereof. In other embodiments, dysbiosis or a dysbiosis-related disease may be treated prophylactically, e.g. if a subject is at risk of developing a dysbiosis or a dysbiosis-related disease, for example if a dysbiosis-inducing agent is administered to the subject. Accordingly, the ATP hydrolyzing enzyme, the nucleic acid, the host cell, the microorganism or the viral particle (for treatment of dysbiosis or a dysbiosis-related disease) may be administered in combination with a dysbiosis-inducing agent, such that the (side) effect of said agent of inducing a dysbiosis is inhibited or reduced.

In certain embodiments, the ATP hydrolyzing enzyme, the nucleic acid, the host cell, the microorganism or the viral particle (for treatment of dysbiosis or a dysbiosis-related disease) may be administered after the end of the administration of the dysbiosis-inducing agent. In particular, the administration of the ATP hydrolyzing enzyme, the nucleic acid, the host cell, the microorganism or the viral particle (for treatment of dysbiosis or a dysbiosis-related disease) may be started, when the dysbiosis-inducing agent no longer exerts its primary pharmacological effects.

For example, dysbiosis induced by antibiotics, chemotherapeutics or other medications typically continues for a long time, even after the dysbiosis-inducing agent no longer induces its primary effects (as antibiotic, chemotherapeutic or other medication). In such a case, the ATP hydrolyzing enzyme, the nucleic acid, the host cell, the microorganism or the viral particle (for treatment of dysbiosis or a dysbiosis-related disease) is not “combined” with the dysbiosis-inducing agent, because the effective time windows of the dysbiosis-inducing agent and the ATP hydrolyzing enzyme, the nucleic acid, the host cell, the microorganism or the viral particle (for treatment of dysbiosis or a dysbiosis-related disease) do not overlap. Thereby, the effects of the ATP hydrolyzing enzyme, the nucleic acid, the host cell, the microorganism or the viral particle (for treatment of dysbiosis or a dysbiosis-related disease) do not interfere with the (other) pharmacological effects of the dysbiosis-inducing agent (i.e., the pharmacological effects of the dysbiosis-inducing agent other than the induction of a dysbiosis). Dysbiosis-inducing agents are typically not administered for the purpose of inducing a dysbiosis, but for other pharmacological effects—while induction of dysbiosis is typically an undesired side effect.

In general, dysbiosis may be induced by any factor for inducing dysbiosis, including external factors (such as administration of dysbiosis-inducing agents, e.g., antibiotics or chemotherapeutic agents), diets, physical and psychological stress as well as endogenous/host-related factors. Dysbiosis may be due to dietary disorders (a hyperprotein hyperlipid diet, rich in sugars and low in fiber; food allergies; malabsorption and impaired digestion of carbohydrates), poor digestive secretions, stress, antibiotic/pharmacological therapy, weakened immune functions, malabsorption, intestinal infections and alterations of the pH in the gastrointestinal tract. In some embodiments, dysbiosis may be induced by a dysbiosis-inducing agent as described herein (such as an antibiotic agent or a chemotherapeutic agent), by a diet or by maternal dysbiosis.

Maternal dysbiosis may have a major impact on the offspring. Accordingly, the ATP hydrolyzing enzyme, the nucleic acid, the host cell, the microorganism or the viral particle as described herein may also be used for the treatment of newborns and infants of mothers suffering from dysbiosis. In some embodiments, this includes newborns and infants of up to one year of age (for human infants). Accordingly, the ATP hydrolyzing enzyme, the nucleic acid, the host cell, the microorganism, the bacterium, or the viral particle may be administered to newborns or infants up to one year. In some embodiments, this includes newborns and infants during (and up to four weeks after the end of) breast feeding. Accordingly, the ATP hydrolyzing enzyme, the nucleic acid, the host cell, the microorganism, the bacterium, or the viral particle may be administered to newborns or infants during (and up to four weeks after the end of) breast feeding.

In particular, the ATP hydrolyzing enzyme, the nucleic acid, the host cell, the microorganism, the bacterium, or the viral particle as described herein may be used for restoring or improving/increasing the microbiome balance during or after dysbiosis, e.g. dysbiosis induced by antibiotic or chemotherapeutic treatment, diet or maternal dysbiosis (in newborns and infants up to one year).

In some embodiments, the ATP hydrolyzing enzyme, the nucleic acid, the host cell, the microorganism, the bacterium, or the viral particle (for use) as described herein may be used for reducing, inhibiting, preventing, ameliorating or decreasing the risk of dysbiosis-induced effects of infection with a pathogen. Accordingly, the present invention also provides a method for reducing the risk of occurrence of, treating, ameliorating, inhibiting or decreasing dysbiosis-induced effects of an infection with a pathogen, comprising administering to the subject
(a) an ATP hydrolyzing enzyme, in particular the ATP hydrolyzing enzyme as described herein;
(b) a nucleic acid comprising a polynucleotide encoding the ATP hydrolyzing enzyme, in particular the nucleic acid as described herein;
(c) a host cell comprising the nucleic acid, in particular the host cell as described herein;
(d) a microorganism comprising the nucleic acid, in particular the microorganism as described herein; or
(e) a viral particle comprising the nucleic acid, in particular the viral particle as described herein.

Preferably, the infection is a bacterial infection, such as infection with Citrobacter rodentium or Clostridioides difficile.

In some embodiments, the ATP hydrolyzing enzyme, the nucleic acid, the host cell, the microorganism, the bacterium, or the viral particle (for use) as described herein may be used for reducing, inhibiting, preventing, ameliorating or decreasing the risk of dysbiosis-induced effects of hypoglycemia and/or weight loss. Accordingly, the present invention also provides a method for reducing the risk of occurrence of, treating, ameliorating, inhibiting or decreasing dysbiosis-induced effects of hypoglycemia and/or weight loss, comprising administering to the subject
(a) an ATP hydrolyzing enzyme, in particular the ATP hydrolyzing enzyme as described herein;
(b) a nucleic acid comprising a polynucleotide encoding the ATP hydrolyzing enzyme, in particular the nucleic acid as described herein;
(c) a host cell comprising the nucleic acid, in particular the host cell as described herein;
(d) a microorganism comprising the nucleic acid, in particular the microorganism as described herein; or
(e) a viral particle comprising the nucleic acid, in particular the viral particle as described herein.

In some embodiments, the ATP hydrolyzing enzyme, the nucleic acid, the host cell, the microorganism, the bacterium, or the viral particle (for use) as described herein may be used for reducing, inhibiting, preventing, ameliorating or decreasing the risk of dysbiosis-induced decrease of microbiota diversity. Accordingly, the present invention also provides a method for reducing the risk of occurrence of, treating, ameliorating, inhibiting or decreasing dysbiosis-induced decrease of microbiota diversity, comprising administering to the subject
(a) an ATP hydrolyzing enzyme, in particular the ATP hydrolyzing enzyme as described herein;
(b) a nucleic acid comprising a polynucleotide encoding the ATP hydrolyzing enzyme, in particular the nucleic acid as described herein;
(c) a host cell comprising the nucleic acid, in particular the host cell as described herein;
(d) a microorganism comprising the nucleic acid, in particular the microorganism as described herein; or
(e) a viral particle comprising the nucleic acid, in particular the viral particle as described herein.

In some embodiments, the ATP hydrolyzing enzyme, the nucleic acid, the host cell, the microorganism, the bacterium, or the viral particle (for use) as described herein may be used for reducing, inhibiting, preventing, ameliorating or decreasing the risk of dysbiosis-induced intestinal bacterial translocation. Accordingly, the present invention also provides a method for reducing the risk of occurrence of, treating, ameliorating, inhibiting or decreasing dysbiosis-induced intestinal bacterial translocation, comprising administering to the subject
(a) an ATP hydrolyzing enzyme, in particular the ATP hydrolyzing enzyme as described herein;
(b) a nucleic acid comprising a polynucleotide encoding the ATP hydrolyzing enzyme, in particular the nucleic acid as described herein;
(c) a host cell comprising the nucleic acid, in particular the host cell as described herein;
(d) a microorganism comprising the nucleic acid, in particular the microorganism as described herein; or
(e) a viral particle comprising the nucleic acid, in particular the viral particle as described herein.

In some embodiments, the ATP hydrolyzing enzyme, the nucleic acid, the host cell, the microorganism, the bacterium, or the viral particle (for use) as described herein may be used for reducing, inhibiting, preventing, ameliorating or decreasing the risk of dysbiosis-induced caecum enlargement. Accordingly, the present invention also provides a method for reducing the risk of occurrence of, treating, ameliorating, inhibiting or decreasing dysbiosis-induced caecum enlargement, comprising administering to the subject
(a) an ATP hydrolyzing enzyme, in particular the ATP hydrolyzing enzyme as described herein;
(b) a nucleic acid comprising a polynucleotide encoding the ATP hydrolyzing enzyme, in particular the nucleic acid as described herein;
(c) a host cell comprising the nucleic acid, in particular the host cell as described herein;
(d) a microorganism comprising the nucleic acid, in particular the microorganism as described herein; or
(e) a viral particle comprising the nucleic acid, in particular the viral particle as described herein.

In some embodiments, the ATP hydrolyzing enzyme, the nucleic acid, the host cell, the microorganism, the bacterium, or the viral particle (for use) as described herein may be used for reducing, inhibiting, preventing, ameliorating or decreasing the risk of dysbiosis-induced (chronic) inflammation of the gut. Accordingly, the present invention also provides a method for reducing the risk of occurrence of, treating, ameliorating, inhibiting or decreasing dysbiosis-induced (chronic) inflammation of the gut, comprising administering to the subject
(a) an ATP hydrolyzing enzyme, in particular the ATP hydrolyzing enzyme as described herein;
(b) a nucleic acid comprising a polynucleotide encoding the ATP hydrolyzing enzyme, in particular the nucleic acid as described herein;
(c) a host cell comprising the nucleic acid, in particular the host cell as described herein;
(d) a microorganism comprising the nucleic acid, in particular the microorganism as described herein; or
(e) a viral particle comprising the nucleic acid, in particular the viral particle as described herein.

In some embodiments, the ATP hydrolyzing enzyme, the nucleic acid, the host cell, the microorganism, the bacterium, or the viral particle (for use) as described herein may be used for reducing, inhibiting, preventing, ameliorating or decreasing the risk of dysbiosis-induced disruption of the intestinal barrier. Accordingly, the present invention also provides a method for reducing the risk of occurrence of, treating, ameliorating, inhibiting or decreasing dysbiosis-induced disruption of the intestinal barrier, comprising administering to the subject
(a) an ATP hydrolyzing enzyme, in particular the ATP hydrolyzing enzyme as described herein;
(b) a nucleic acid comprising a polynucleotide encoding the ATP hydrolyzing enzyme, in particular the nucleic acid as described herein;
(c) a host cell comprising the nucleic acid, in particular the host cell as described herein;
(d) a microorganism comprising the nucleic acid, in particular the microorganism as described herein; or
(e) a viral particle comprising the nucleic acid, in particular the viral particle as described herein.

In some embodiments, the ATP hydrolyzing enzyme, the nucleic acid, the host cell, the microorganism, the bacterium, or the viral particle (for use) as described herein may be used for reducing, inhibiting, preventing, ameliorating or decreasing the risk of dysbiosis-induced impairment of metabolic functions. Accordingly, the present invention also provides a method for reducing the risk of occurrence of, treating, ameliorating, inhibiting or decreasing dysbiosis-induced impairment of metabolic functions, comprising administering to the subject
(a) an ATP hydrolyzing enzyme, in particular the ATP hydrolyzing enzyme as described herein;
(b) a nucleic acid comprising a polynucleotide encoding the ATP hydrolyzing enzyme, in particular the nucleic acid as described herein;
(c) a host cell comprising the nucleic acid, in particular the host cell as described herein;
(d) a microorganism comprising the nucleic acid, in particular the microorganism as described herein; or
(e) a viral particle comprising the nucleic acid, in particular the viral particle as described herein.

Metabolic functions, which may be reduced or impaired due to dysbiosis (and therefore treated as described above) include insulin resistance, hepatic fat deposition, adipose tissue development, rheumatic arthritis and ulcerative colitis.

Combination with a Dysbiosis-Inducing Agent

Under certain circumstances, dysbiosis-inducing agents are administered, for example to treat certain diseases and conditions. Non-limiting examples of dysbiosis-inducing agents include dysbiosis-inducing antibiotics and chemotherapeutic agents, but also oral iron supplementation, as well as other dysbiosis-inducing medications. As shown in the appended examples, dysbiosis can be reduced or avoided, if the dysbiosis-inducing agent is administered in combination with an ATP-hydrolyzing enzyme or a host cell/microorganism comprising a nucleic acid encoding an ATP-hydrolyzing enzyme. Accordingly, the ATP hydrolyzing enzyme, the nucleic acid comprising the polynucleotide encoding the ATP hydrolyzing enzyme, the host cell comprising the nucleic acid comprising the polynucleotide encoding the ATP hydrolyzing enzyme, the microorganism comprising the nucleic acid comprising the polynucleotide encoding the ATP hydrolyzing enzyme, or the viral particle comprising the nucleic acid comprising the polynucleotide encoding the ATP hydrolyzing enzyme may be combined with a dysbiosis-inducing agent. It is understood that in such a combination, the ATP hydrolyzing enzyme, the nucleic acid comprising the polynucleotide encoding the ATP hydrolyzing enzyme, the host cell comprising the nucleic acid comprising the polynucleotide encoding the ATP hydrolyzing enzyme, the microorganism comprising the nucleic acid comprising the polynucleotide encoding the ATP hydrolyzing enzyme, or the viral particle comprising the nucleic acid comprising the polynucleotide encoding the ATP hydrolyzing enzyme is used to counteract, reduce, ameliorate, decrease, inhibit or to reduce the risk of dysbiosis induced by the dysbiosis-inducing agent.

Therefore, the present invention also provides a combination of
(i) an ATP-hydrolyzing enzyme as described herein; and
(ii) a dysbiosis-inducing agent.

The present invention also provides a combination of
(i) a nucleic acid as described herein comprising a polynucleotide encoding an ATP-hydrolyzing enzyme; and
(ii) a dysbiosis-inducing agent.

The present invention also provides a combination of
(i) a host cell as described herein comprising a nucleic acid comprising a polynucleotide encoding an ATP-hydrolyzing enzyme; and
(ii) a dysbiosis-inducing agent.

The present invention also provides a combination of
(i) a microorganism as described herein comprising a nucleic acid comprising a polynucleotide encoding an ATP-hydrolyzing enzyme; and
(ii) a dysbiosis-inducing agent.

The present invention also provides a combination of
(i) a viral particle as described herein comprising a nucleic acid comprising a polynucleotide encoding an ATP-hydrolyzing enzyme; and
(ii) a dysbiosis-inducing agent.

In particular, the present invention also provides a combination of
(i) a bacterium as described herein comprising a nucleic acid comprising a polynucleotide encoding an ATP-hydrolyzing enzyme; and
(ii) a dysbiosis-inducing agent.

In particular, the bacterium is a recombinant bacterium, which expresses the ATP-hydrolyzing enzyme, preferably apyrase, heterologously.

Accordingly, the present invention provides a combination of
(i) a) an ATP-hydrolyzing enzyme as described herein,
   b) a nucleic acid as described herein comprising a polynucleotide encoding an ATP-hydrolyzing enzyme,
   c) a host cell as described herein comprising a nucleic acid comprising a polynucleotide encoding an ATP-hydrolyzing enzyme;
   d) a microorganism as described herein comprising a nucleic acid comprising a polynucleotide encoding an ATP-hydrolyzing enzyme;
   e) a viral particle as described herein comprising a nucleic acid comprising a polynucleotide encoding an ATP-hydrolyzing enzyme; or
   f) a bacterium as described herein comprising a nucleic acid comprising a polynucleotide encoding an ATP-hydrolyzing enzyme; and
(ii) a dysbiosis-inducing agent

for use in the treatment of dysbiosis or a dysbiosis-related disease.

The detailed description of the ATP-hydrolyzing enzyme, the nucleic acid comprising the polynucleotide encoding the ATP-hydrolyzing enzyme, the host cell, microorganism or viral particle comprising the polynucleotide encoding the ATP-hydrolyzing enzyme (e.g., the bacterium) as provided above applies accordingly for the combination with the dysbiosis-inducing agent. Moreover, the ATP hydrolyzing enzyme, the nucleic acid comprising the polynucleotide encoding the ATP hydrolyzing enzyme, the host cell comprising the nucleic acid comprising the polynucleotide encoding the ATP hydrolyzing enzyme, the microorganism comprising the nucleic acid comprising the polynucleotide encoding the ATP hydrolyzing enzyme, or the viral particle comprising the nucleic acid comprising the polynucleotide encoding the ATP hydrolyzing enzyme may be comprised in a composition as described above. Similarly, the dysbiosis-inducing agent may be comprised in a composition. The detailed description of the composition as provided above applies accordingly to a composition comprising the dysbiosis-inducing agent. Furthermore, the combination may be used in medicine, in particular for the treatment of dysbiosis or a dysbiosis-related disease, as described herein.

Dysbiosis-inducing agents are described, for example, in Le Bastard Q, Al-Ghalith G A, Grëgoire M, et al. Systematic review: human gut dysbiosis induced by non-antibiotic prescription medications. Aliment Pharmacol Ther. 2018; 47(3):332-345. doi:10.1111/apt.14451. Dysbiosis-inducing agents include dysbiosis-inducing antibiotics, chemotherapeutic agents, proton pump inhibitors, statins, immunosuppressive drugs (e.g., glucocorticoids), metformin, antipsychotics (e.g., atypical antipsychotics) and agents for oral iron supplementation.

In some embodiments, the dysbiosis-inducing agent may be an antibiotic. In other embodiments, the dysbiosis-inducing agent may be a non-antibiotic medication. Examples of dysbiosis-inducing non-antibiotic medications include chemotherapeutic agents, proton pump inhibitors, statins, immunosuppressive drugs (e.g., glucocorticoids), metformin, and antipsychotics (e.g., atypical antipsychotics). Preferably, the dysbiosis-inducing non-antibiotic medication is a chemotherapeutic agent or a proton pump inhibitor. In particular, the dysbiosis-inducing chemotherapeutic agent may be a cytotoxic or cytostatic agent. In some embodiments, the dysbiosis-inducing chemotherapeutic agent may be selected from the group consisting of alkylating agents, anthracyclines, cytoskeletal disruptors, epothilones, histone deacetylase inhibitors, inhibitors of topoisomerase I or II, kinase inhibitors, nucleotide analogs and precursor analogs, platinum-based agents, retinoids, and vinca alkaloids and derivatives. Specific, non-limiting examples of dysbiosis-inducing chemotherapeutic agents include 5-fluorouracil (5-FU) and irinotecan.

The dysbiosis-inducing antibiotic may be selected from the group consisting of penicillins, tetracyclines, cephalosporins, quinolones, lincosamides, macrolides, sulfonamides, glycopeptides, aminoglycosides, carbapenems, ansamycins, carbacephems, lipopeptides, monobactams, nitrofurans, oxazolidinones, and polypeptides.

In some embodiments, the antibiotic may be an aminoglycoside. Non-limiting examples of aminoglycosides include amikacin, gentamicin, kanamycin, neomycin, netilmicin, tobramycin, paromomycin, streptomycin, and spectinomycin.

In some embodiments, the antibiotic may be an ansamycin. Non-limiting examples of ansamycins include geldanamycin, herbimycin, and rifaximin.

In some embodiments, the antibiotic may be a carbacephem, such as loracarbef.

In some embodiments, the antibiotic may be a carbapenem. Non-limiting examples of carbapenems include ertapenem, doripenem, imipenem/cilastatin, and meropenem.

In some embodiments, the antibiotic may be a cephalosporin (e.g., first, second, third, fourth or fifth generation). Non-limiting examples of first-generation cephalosporins include cefadroxil, cefazolin, cephradine, cephapirin, cephalothin, and cefalexin. Non-limiting examples of second-generation cephalosporins include cefaclor, cefoxitin, cefotetan, cefamandole, cefmetazole, cefonicid, loracarbef, cefprozil, and cefuroxime. Non-limiting examples of third-generation cephalosporins include cefixime, cefdinir, cefditoren, cefoperazone, cefotaxime, cefpodoxime, ceftazidime, ceftibuten, ceftizoxime, moxalactam, and ceftriaxone. Non-limiting examples of fourth-generation cephalosporins include cefepime. Non-limiting examples of fifth-generation cephalosporins include ceftaroline, fosamil and ceftobiprole.

In some embodiments, the antibiotic may be a glycopeptide antibiotic. Non-limiting examples of glycopeptide antibiotics include teicoplanin, vancomycin, telavancin, dalbavancin, and oritavancin.

In some embodiments, the antibiotic may be a lincosamide. Non-limiting examples of lincosamides include clindamycin and lincomycin.

In some embodiments, the antibiotic may be a lipopeptide antibiotic, such as daptomycin.

In some embodiments, the antibiotic may be a macrolide. Non-limiting examples of macrolides include azithromycin, clarithromycin, erythromycin, roxithromycin, telithromycin, spiramycin, and fidaxomicin.

In some embodiments, the antibiotic may be a monobactam, such as aztreonam.

In some embodiments, the antibiotic may be a nitrofuran. Non-limiting examples of nitrofurans include furazolidone and nitrofurantoin.

In some embodiments, the antibiotic may be an oxazolidinone. Non-limiting examples of oxazolidinones include linezolid, posizolid, radezolid and torezolid.

In some embodiments, the antibiotic may be a penicillin. Non-limiting examples of penicillins include amoxicillin, ampicillin, azlocillin, dicloxacillin, flucloxacillin, mezlocillin, methicillin, nafcillin, oxacillin, penicillin G, penicillin V, piperacillin, penicillin G, temocillin, and ticarcillin.

In some embodiments, the antibiotic may be a polypeptide antibiotic. Non-limiting examples of polypeptide antibiotics include bacitracin, colistin and polymyxin B.

In some embodiments, the antibiotic may be a quinolone/fluoroquinolone. Non-limiting examples of quinolones/fluoroquinolones include ciprofloxacin, enoxacin, gatifloxacin, gemifloxacin, levofloxacin, lomefloxacin, moxifloxacin, nadifloxacin, nalidixic acid, norfloxacin, ofloxacin, trovafloxacin, grepafloxacin, sparfloxacin, and temafloxacin.

In some embodiments, the antibiotic may be a sulfonamide. Non-limiting examples of sulfonamides include mafenide, sulfacetamide, sulfadiazine, silver sulfadiazine, sulfadimethoxine, sulfamethizole, sulfamethoxazole, sulfanilimide, sulfasalazine, sulfisoxazole, trimethoprim-sulfamethoxazole, and sulfonamidochrysoidine.

In some embodiments, the antibiotic may be a tetracyclin. Non-limiting examples of tetracyclins include demeclocycline, doxycycline, metacycline, minocycline, oxytetracycline, and tetracycline.

Accordingly, non-limiting examples of antibiotics include amikacin, gentamicin, kanamycin, neomycin, netilmicin, tobramycin, paromomycin, streptomycin, spectinomycin, geldanamycin, herbimycin, rifaximin, loracarbef, ertapenem, doripenem, imipenem/cilastatin, meropenem, cefadroxil, cefazolin, cephradine, cephapirin, cephalothin, cefalexin, cefaclor, cefoxitin, cefotetan, cefamandole, cefmetazole, cefonicid, loracarbef, cefprozil, cefuroxime, cefixime, cefdinir, cefditoren, cefoperazone, cefotaxime, cefpodoxime, ceftazidime, ceftibuten, ceftizoxime, moxalactam, ceftriaxone, cefepime, ceftaroline fosamil, ceftobiprole, teicoplanin, vancomycin, telavancin, dalbavancin, oritavancin, clindamycin, lincomycin, daptomycin, azithromycin, clarithromycin, erythromycin, roxithromycin, telithromycin, spiramycin, fidaxomicin, aztreonam, furazolidone, nitrofurantoin, linezolid, posizolid, radezolid, torezolid, amoxicillin, ampicillin, azlocillin, dicloxacillin, flucloxacillin, mezlocillin, methicillin, nafcillin, oxacillin, penicillin G, penicillin V, piperacillin, penicillin G, temocillin, ticarcillin, bacitracin, colistin, polymyxin B, ciprofloxacin, enoxacin, gatifloxacin, gemifloxacin, levofloxacin, lomefloxacin, moxifloxacin, nadifloxacin, nalidixic acid, norfloxacin, ofloxacin, trovafloxacin, grepafloxacin, sparfloxacin, temafloxacin, mafenide, sulfacetamide, sulfadiazine, silver sulfadiazine, sulfadimethoxine, sulfamethizole, sulfamethoxazole, sulfanilimide, sulfasalazine, sulfisoxazole, trimethoprim-sulfamethoxazole, sulfonamidochrysoidine, demeclocycline, doxycycline, metacycline, minocycline, oxytetracycline, tetracycline, arsphenamine, chloramphenicol, fosfomycin, fusidic acid, metronidazole, mupirocin, platensimycin, quinupristin/dalfopristin, thiamphenicol, tigecycline, tinidazole, and trimethoprim.

In some embodiments, the antibiotic may be a penicillin, such as ampicillin. In some embodiments, the antibiotic may be a (third generation) cephalosporin, such as cefoperazone. In some instances, the antibiotic may be a glycopeptide-antibiotic, such as vancomycin. In certain embodiments, the antibiotic may be metronidazole. Particularly preferably, the antibiotic may be selected from the group consisting of vancomycin, ampicillin, metronidazole and cefoperazone; in particular the antibiotic may be ampicillin or cefoperazone.

In some embodiments, the ATP hydrolyzing enzyme, the nucleic acid comprising the polynucleotide encoding the ATP hydrolyzing enzyme, the host cell comprising the nucleic acid comprising the polynucleotide encoding the ATP hydrolyzing enzyme, the microorganism comprising the nucleic acid comprising the polynucleotide encoding the ATP hydrolyzing enzyme, or the viral particle comprising the nucleic acid comprising the polynucleotide encoding the ATP hydrolyzing enzyme is not administered in combination with an antibiotic.

In general, the dysbiosis-inducing agent may be combined with the ATP-hydrolyzing enzyme, a nucleic acid encoding the ATP-hydrolyzing enzyme, or a host cell, microorganism or viral particle comprising the nucleic acid encoding the ATP-hydrolyzing enzyme as described herein. The ATP-hydrolyzing enzyme encoded by the nucleic acid may be expressed, such that at the place of action (e.g., in the human or animal body), where the combination exerts its effects, the dysbiosis-inducing agent is combined with the ATP-hydrolyzing enzyme.

In general, a “combination” of (i) the dysbiosis-inducing agent as described herein and of (ii) the ATP-hydrolyzing enzyme, a nucleic acid encoding the ATP-hydrolyzing enzyme, or a host cell, microorganism or viral particle comprising the nucleic acid encoding the ATP-hydrolyzing enzyme, as described herein, means that both components can exert their effects in a combined manner. To this end, the time window of the effects of both components usually overlaps. Accordingly, the effects of both components are usually present in the human or animal body at the same time (even if one or both of the components may be no longer physically present). In some embodiments, both components may be (physically) present in the human or animal body at the same time.

Accordingly, (i) the treatment with the dysbiosis-inducing agent as described herein may overlap with (ii) the treatment with the ATP-hydrolyzing enzyme, a nucleic acid encoding the ATP-hydrolyzing enzyme, or a host cell, microorganism or viral particle comprising the nucleic acid encoding the ATP-hydrolyzing enzyme, as described herein. Even if one component (i) or (ii) may not be administered, e.g., at the same day, as the other component (the other of (i) or (ii)), their treatment schedules are usually intertwined. This means that “a combination” in the context of the present invention does in particular not include the start of a treatment with one component (i) or (ii), when the treatment with the other component of the components (i) and (ii) is already finished.

In some embodiments the first administration of the ATP-hydrolyzing enzyme, the nucleic acid encoding the ATP-hydrolyzing enzyme, or the host cell, microorganism or viral particle comprising the nucleic acid encoding the ATP-hydrolyzing enzyme may start not more than one week (preferably not more than 3 days, more preferably not more than 2 days, even more preferably not more than a day) after the (final) treatment with the dysbiosis-inducing agent (e.g., the final administration of the dysbiosis-inducing agent). In some embodiments, the first administration of the dysbiosis-inducing agent starts not more than one week (preferably not more than 3 days, more preferably not more than 2 days, even more preferably not more than a day) after the (final) treatment with the ATP-hydrolyzing enzyme, the nucleic acid encoding the ATP-hydrolyzing enzyme, or the host cell, microorganism or viral particle comprising the nucleic acid encoding the ATP-hydrolyzing enzyme (e.g., the final administration of the ATP-hydrolyzing enzyme, the nucleic acid encoding the ATP-hydrolyzing enzyme, or the host cell, microorganism or viral particle comprising the nucleic acid encoding the ATP-hydrolyzing enzyme).

For example, in the combination of (i) the dysbiosis-inducing agent as described herein and of (ii) the ATP-hydrolyzing enzyme, the nucleic acid encoding the ATP-hydrolyzing enzyme, or the host cell, microorganism or viral particle comprising the nucleic acid encoding the ATP-hydrolyzing enzyme, one component ((i) or (ii)) may be administered once or twice a week (e.g., (i) the dysbiosis-inducing agent), while the other component may be administered daily (e.g., (ii) the ATP-hydrolyzing enzyme, the nucleic acid encoding the ATP-hydrolyzing enzyme, or the host cell, microorganism or viral particle comprising the nucleic acid encoding the ATP-hydrolyzing enzyme). In this example, on some days of the daily administration of one component also the other component is administered. However, in another example, if both components were administered weekly, in some of the weeks both components may be administered (even if not administered at the same day, the treatment schedules still overlap). If one of the components is administered only once, while the other component is administered repeatedly, the single administration of one component usually lies within the treatment cycle of the other component (even if not administered at the same day). In other embodiments, both components are administered daily for an overlapping time, i.e. at least at some days (e.g. 1, 2, 3, 4, 5, 6, 7, 8, 9, 10, 11, 12, 13, 14, 15, 16, 17, 18, 19, 20 or more) days both components are administered. In general, to achieve a combination, one component may be administered as long as its effects overlap with the effects of the other component.

The administration of (i) the dysbiosis-inducing agent as described herein and/or of (ii) the ATP-hydrolyzing enzyme, the nucleic acid encoding the ATP-hydrolyzing enzyme, or the host cell, microorganism or viral particle comprising the nucleic acid encoding the ATP-hydrolyzing enzyme may require repeated (multiple, i.e. more than one) administrations, e.g. multiple injections and/or multiple oral administrations. Thus, the administration may be repeated at least two times, or, e.g., in a daily manner. Accordingly, (i) the dysbiosis-inducing agent as described herein and (ii) the ATP-hydrolyzing enzyme, the nucleic acid encoding the ATP-hydrolyzing enzyme, or the host cell, microorganism or viral particle comprising the nucleic acid encoding the ATP-hydrolyzing enzyme may be administered repeatedly or continuously. The dysbiosis-inducing agent as described herein and the ATP-hydrolyzing enzyme, the nucleic acid encoding the ATP-hydrolyzing enzyme, or the host cell, microorganism or viral particle comprising the nucleic acid encoding the ATP-hydrolyzing enzyme may be administered repeatedly or continuously for a period of at least 1, 2, 3, or 4 weeks; 2, 3, 4, 5, 6, 8, 10, or 12 months; or 2, 3, 4, or 5 years. For example, the dysbiosis-inducing agent modulator may be administered twice per day, once per day, every two days, every three days, once per week, every two weeks, every three weeks, once per month or every two months. For example, the ATP-hydrolyzing enzyme, the nucleic acid encoding the ATP-hydrolyzing enzyme, or the host cell, microorganism or viral particle comprising the nucleic acid encoding the ATP-hydrolyzing enzyme may be administered twice per day, once per day, every two days, every three days, once per week, every two weeks, every three weeks, once per month or every two months.

In some embodiments, (i) the dysbiosis-inducing agent; and/or (ii) the ATP hydrolyzing enzyme, the nucleic acid, the host cell, the microorganism or the viral particle are administered on the same day. In some embodiments, (i) the dysbiosis-inducing agent; and/or (ii) the ATP hydrolyzing enzyme, the nucleic acid, the host cell, the microorganism or the viral particle are administered repeatedly. For example, the ATP hydrolyzing enzyme, the nucleic acid, the host cell, the microorganism or the viral particle may be administered daily, while the dysbiosis-inducing agent may be administered once or twice a week on days, on which also the other component (the ATP hydrolyzing enzyme, the nucleic acid, the host cell, the microorganism or the viral particle) is administered.

In some embodiments, (i) the dysbiosis-inducing agent; and (ii) the ATP hydrolyzing enzyme, the nucleic acid, the host cell, the microorganism or the viral particle may be administered at about the same time. “At about the same time”, as used herein, means in particular simultaneous administration or that directly after administration of component (i) component (ii) is administered or vice versa. The skilled person understands that “directly after” includes the time necessary to prepare the second administration —for example the time necessary for exposing and disinfecting the location for the second administration as well as appropriate preparation of the “administration device” (e.g., syringe, pump, etc.). Simultaneous administration also includes if the periods of administration of both components overlap or if, for example, one component is administered over a longer period of time, such as 30 min, 1 h, 2 h or even more, e.g. by infusion, and the other component is administered at some time during such a long period.

Preferably, (i) the dysbiosis-inducing agent; and (ii) the ATP hydrolyzing enzyme, the nucleic acid, the host cell, the microorganism or the viral particle are administered consecutively. More preferably, (i) the dysbiosis-inducing agent may be administered before (ii) the ATP hydrolyzing enzyme, the nucleic acid, the host cell, the microorganism or the viral particle is administered. Alternatively, (i) the dysbiosis-inducing agent may be administered after (ii) the ATP hydrolyzing enzyme, the nucleic acid, the host cell, the microorganism or the viral particle is administered. In consecutive administration, the time interval between administration of both components (i) and (ii) is preferably no more than one week, more preferably no more than 3 days, even more preferably no more than 2 days and most preferably no more than 24 h are in between administration of both components (i) and (ii). It is particularly preferred that (i) the dysbiosis-inducing agent; and (ii) the ATP hydrolyzing enzyme, the nucleic acid, the host cell, the microorganism or the viral particle are administered at the same day. The time between administration of both components (i) and (ii) may be no more than 12 hours, preferably no more than 6 hours, more preferably no more than 3 hours, e.g. no more than 2 hours or no more than 1 hour.

The dysbiosis-inducing agent and the ATP hydrolyzing enzyme, the nucleic acid, the host cell, the microorganism or the viral particle can be administered by various routes of administration, for example, systemically or locally. Routes for systemic administration in general include, for example, enteral and parenteral routes, which include subcutaneous, intravenous, intramuscular, intraarterial, intradermal and intraperitoneal routes. The dysbiosis-inducing agent and the ATP hydrolyzing enzyme, the nucleic acid, the host cell, the microorganism or the viral particle may be administered via the same or distinct routes of administration.

As described above, the ATP hydrolyzing enzyme, the nucleic acid, the host cell, the microorganism or the viral particle are preferably administered via an enteral route of administration. Likewise, the dysbiosis-inducing agent may be administered via an enteral route of administration. Enteral routes of administration include, for example oral, sublingual, and rectal administration as well as administration via a gastric tube. Oral administration may be preferred. However, the dysbiosis-inducing agent may also be administered via a parenteral route of administration (e.g., while the ATP hydrolyzing enzyme, the nucleic acid, the host cell, the microorganism or the viral particle is administered via an enteral route of administration). Non-limiting examples of parental administration include intravenous, intraarterial, intramuscular, intradermal, intranodal, intraperitoneal, and subcutaneous routes of administration. In some embodiments, the dysbiosis-inducing agent may be administered intravenously or subcutaneously.

In certain embodiments, (i) the dysbiosis-inducing agent; and (ii) the ATP hydrolyzing enzyme, the nucleic acid, the host cell, the microorganism or the viral particle are administered via the same route of administration, such as any one of the enteral or parental route described above.

The dysbiosis-inducing agent and the ATP hydrolyzing enzyme, the nucleic acid, the host cell, the microorganism or the viral particle may be provided in the same or in distinct compositions. Preferably, (i) the dysbiosis-inducing agent and (ii) the ATP hydrolyzing enzyme, the nucleic acid, the host cell, the microorganism or the viral particle as described above are provided in distinct compositions, e.g. as described above. Thereby, different other components, e.g. different vehicles, can be used for (i) the dysbiosis-inducing agent and (ii) the ATP hydrolyzing enzyme, the nucleic acid, the host cell, the microorganism or the viral particle as described above. Moreover, (i) the dysbiosis-inducing agent and (ii) the ATP hydrolyzing enzyme, the nucleic acid, the host cell, the microorganism or the viral particle as described above can be administered via different routes of administration and the doses (in particular the relation of the doses) can be adjusted according to the actual need.

Kits

The present invention also provides a kit comprising
(i) an ATP-hydrolyzing enzyme as described herein; and
(ii) a dysbiosis-inducing agent.

The present invention also provides a kit comprising
(i) a nucleic acid as described herein comprising a polynucleotide encoding an ATP-hydrolyzing enzyme; and
(ii) a dysbiosis-inducing agent.

The present invention also provides a kit comprising
(i) a host cell as described herein comprising a nucleic acid comprising a polynucleotide encoding an ATP-hydrolyzing enzyme; and
(ii) a dysbiosis-inducing agent.

The present invention also provides a kit comprising
(i) a microorganism as described herein comprising a nucleic acid comprising a polynucleotide encoding an ATP-hydrolyzing enzyme; and
(ii) a dysbiosis-inducing agent.

The present invention also provides a kit comprising
(i) a viral particle as described herein comprising a nucleic acid comprising a polynucleotide encoding an ATP-hydrolyzing enzyme; and
(ii) a dysbiosis-inducing agent.

In particular, the present invention also provides a kit comprising
(i) a bacterium as described herein comprising a nucleic acid comprising a polynucleotide encoding an ATP-hydrolyzing enzyme; and
(ii) a dysbiosis-inducing agent.

In particular, the bacterium is a recombinant bacterium, which expresses the ATP-hydrolyzing enzyme, preferably apyrase, heterologously.

Accordingly, the present invention provides a kit comprising
(i) a) an ATP-hydrolyzing enzyme as described herein,
   b) a nucleic acid as described herein comprising a polynucleotide encoding an ATP-hydrolyzing enzyme,
   c) a host cell as described herein comprising a nucleic acid comprising a polynucleotide encoding an ATP-hydrolyzing enzyme;
   d) a microorganism as described herein comprising a nucleic acid comprising a polynucleotide encoding an ATP-hydrolyzing enzyme;
   e) a viral particle as described herein comprising a nucleic acid comprising a polynucleotide encoding an ATP-hydrolyzing enzyme; or
   f) a bacterium as described herein comprising a nucleic acid comprising a polynucleotide encoding an ATP-hydrolyzing enzyme; and
(ii) a dysbiosis-inducing agent as described herein

for use in the treatment of dysbiosis or a dysbiosis-related disease.

The detailed description of the ATP-hydrolyzing enzyme, the nucleic acid comprising the polynucleotide encoding the ATP-hydrolyzing enzyme, the host cell, microorganism or viral particle comprising the polynucleotide encoding the ATP-hydrolyzing enzyme (e.g., the bacterium) as provided above applies accordingly for the kit. Likewise, the detailed description of the dysbiosis-inducing agent as provided above applies accordingly for the kit. Moreover, the ATP hydrolyzing enzyme, the nucleic acid comprising the polynucleotide encoding the ATP hydrolyzing enzyme, the host cell comprising the nucleic acid comprising the polynucleotide encoding the ATP hydrolyzing enzyme, the microorganism comprising the nucleic acid comprising the polynucleotide encoding the ATP hydrolyzing enzyme, or the viral particle comprising the nucleic acid comprising the polynucleotide encoding the ATP hydrolyzing enzyme may be comprised in a composition as described above. Similarly, the dysbiosis-inducing agent may be comprised in a composition as described above. The detailed description of the composition as provided above applies accordingly to a composition comprising the dysbiosis-inducing agent. Furthermore, the kit may be used in medicine, in particular for the treatment of dysbiosis or a dysbiosis-related disease, as described herein.

In some embodiments, such a kit comprises (i) the dysbiosis-inducing agent as described above and (ii) an ATP-hydrolyzing enzyme as described above. In some embodiments, such a kit comprises (i) the dysbiosis-inducing agent as described above and (ii) a nucleic acid as described above encoding the ATP-hydrolyzing enzyme. In some embodiments, such a kit comprises (i) the dysbiosis-inducing agent as described above and (ii) a host cell as described above comprising a nucleic acid comprising a polynucleotide encoding the ATP hydrolyzing enzyme. In some embodiments, such a kit comprises (i) the dysbiosis-inducing agent as described above and (ii) a microorganism as described above comprising a nucleic acid comprising a polynucleotide encoding the ATP hydrolyzing enzyme. In some embodiments, such a kit comprises (i) the dysbiosis-inducing agent as described above and (ii) a viral particle as described above comprising a nucleic acid comprising a polynucleotide encoding the ATP hydrolyzing enzyme. Accordingly, the detailed embodiments of the dysbiosis-inducing agent as described above apply accordingly to the kit according to the present invention. Accordingly, the detailed embodiments of the ATP-hydrolyzing enzyme as described above, the nucleic acid as described above encoding the ATP-hydrolyzing enzyme, or the host cell as described above, the microorganism as described above or the viral particle as described above apply accordingly to the kit according to the present invention.

The various components of the kit may be packaged in one or more containers. In some embodiments, the different components; in particular components (i) and (ii), i.e. (i) the dysbiosis-inducing agent as described above and (ii) the ATP hydrolyzing enzyme, the nucleic acid, the host cell, the microorganism or the viral particle as described herein; are provided in distinct containers. The distinct containers with the components may be provided together, e.g. in a box/container. The above components may be provided in a lyophilized or dry form or dissolved in a suitable buffer. For example, the kit may comprise a (pharmaceutical) composition comprising the dysbiosis-inducing agent as described above and a (pharmaceutical) composition comprising any of the ATP-hydrolyzing enzyme as described above, the nucleic acid as described above encoding the ATP-hydrolyzing enzyme, or the host cell as described above, the microorganism as described above or the viral particle as described above, e.g. with each composition in a separate container. The kit may also comprise a (pharmaceutical) composition comprising both, the dysbiosis-inducing agent and any of the ATP-hydrolyzing enzyme as described above, the nucleic acid as described above encoding the ATP-hydrolyzing enzyme, or the host cell as described above, the microorganism as described above or the viral particle as described above.

The kit may also comprise additional reagents including, for instance, buffers for storage and/or reconstitution of the above-referenced components, washing solutions, and the like.

In addition, the kit-of-parts may optionally contain instructions of use. Preferably, the kit further comprises a package insert or label with directions to treat dysbiosis or a dysbiosis-related disease by using a combination of (i) the dysbiosis-inducing agent and (ii) the ATP-hydrolyzing enzyme as described above, the nucleic acid as described above encoding the ATP-hydrolyzing enzyme, or the host cell as described above, the microorganism as described above or the viral particle as described above. For example, the directions to use the combination according to the present invention as described above may include an administration regimen.

### BRIEF DESCRIPTION OF THE FIGURES

In the following a brief description of the appended figures will be given. The figures are intended to illustrate the present invention in more detail. However, they are not intended to limit the subject matter of the invention in any way.

FIG. 1 shows a map of the pHND10 plasmid carrying the phoN2 gene encoding periplasmic ATP-diphosphohydrolase (apyrase).

FIG. 2 shows the amino acid sequence of wild-type phon2 protein (apyrase; SEQ ID NO: 1) and indicates the position of the R192P substitution in the loss-of-function isoform (SEQ ID NO: 2).

FIG. 3 shows the nucleotide sequence of the phoN2 gene (SEQ ID NO: 3) used for generating pHND10 plasmid.

FIG. 4 shows for Example 2 the treatment schedule in a mouse model of antibiotics-induced dysbiosis.

FIG. 5 shows for Example 2 the metagenomic analysis by 16S sequencing of caecal samples from mice treated as described herein. Shannon diversity index at the bacterial family level in caecal samples from control, ABX, ABX+E. coli^{pHND19} and ABX+E. coli^{pAPyr} treated C57BL/6 mice. Means±SEM are shown. Two-tailed Mann-Whitney U-test was used. **p<0.01.

FIG. 6 shows for Example 2 that treatment with bacteria expressing apyrase preserves beta-diversity after induction of dysbiosis. Principle coordinate analysis (PCoA) of bacterial beta-diversity based on an unweighted Unifrac dissimilarity matrix. PERMANOVA was used. p<0.001.

FIG. 7 shows for Example 2 that treatment with bacteria expressing apyrase promotes microbiome recovery from dysbiosis. The heatmap shows bacterial species in caecal microbiota that discriminate the experimental groups: not treated (control); ABX, ABX+E. coli^{pHND19} and ABX+E. coli^{pApyr} treated C57BL/6 mice. Species were selected according to p<0.05 with Wald test using FDR p-value correction following DESeq2 read counts normalization. Each line represents one species, and each column represents an individual mouse. Mean relative abundances (log 10) of species detected in not treated (control), ABX, ABX+E. coli^{pHND19} and ABX+E. coli^{pApyr} are shown.

FIG. 8 shows for Example 2 the p values related to the heatmap shown in FIG. 7, calculated with Wald test using FDR p-value correction following DESeq2 read counts normalization.

FIG. 9 shows for Example 3 the treatment schedule in a mouse model of Citrobacter rodentium infection after induction of dysbiosis.

FIG. 10 shows for Example 3 the percentage body weight variation in mice not treated (control), infected with C. rodentium or pretreated with E. coli^{pHND19} or E. coli^{pApyr} and then infected with C. rodentium. Means±SEM are shown. Two-way ANOVA was used. **p<0.01, ***p<0.001.

FIG. 11 shows for Example 3 the statistical analysis of PMN cells (gated as CD45⁺Gr1⁺CD11b⁺) infiltrates in caecum lamina propria six days after infection, in mice not treated (control), infected with C. rodentium or pretreated with E. coli^{pHND19} or E. coli^{pApyr} and then infected with C. rodentium. Accordingly, PMN cells infiltration in the caecum lamina propria of mice treated with E. coli^{pApyr} and infected with C. rodentium is reduced. Means±SEM are shown. Two-tailed Mann-Whitney U-test was used *p<0.05.

FIG. 12 shows for Example 3 the statistical analysis of inflammatory monocytes (gated as CD45⁺CD11b⁺Ly6c⁺Ly6g⁻) infiltrates in caecum lamina propria 6 days after infection, in mice not treated (control), infected with C. rodentium or pretreated with E. coli^{pHND19} or E. coli^{pApyr} and then infected with C. rodentium. Accordingly, monocytes infiltration in the caecum lamina propria of mice treated with E. coli^{pApyr} and infected with C. rodentium is reduced. Means±SEM are shown. Two-tailed Mann-Whitney U-test was used *p<0.05, **p<0.01.

FIG. 13 shows for Example 4 the treatment schedule in a mouse model of Clostridioides difficile infection after induction of dysbiosis. Dysbiosis was induced by daily oral gavage of ABX for 4 consecutive days. After the antibiotic treatment, during the recovery phase, mice were orally gavaged for 4 days with PBS (control) or 10¹⁰ CFU of E. coli^{pHND19} or E. coli^{PApyr}. At day 4, mice were orally infected with 10⁵ of C. difficile VPI 10463 spores

FIG. 14 shows for Example 4 the percentage body weight loss in mice not treated (control), infected with C. difficile or pretreated with E. coli^{pHND19} or E. coli^{pApyr} and then infected with C. difficile. Accordingly, E. coli^{pApyr} treatment attenuates body weight loss induced by C. difficile intestinal infection. Means±SEM are shown. Two-way ANOVA was used. *p<0.05, ***p<0.001.

FIG. 15 shows for Example 4 the clinical score variations in mice not treated (control), infected with C. difficile or pretreated with E. coli^{pHND19} or E. coli^{pApyr} and then infected with C. difficile. Accordingly, E. coli^{pApyr} treatment ameliorates the clinical score in mice infected with C. difficile. Means±SEM are shown. Two-way ANOVA was used. ***p<0.001

FIG. 16 shows for Example 4 the percent survival of mice not treated (control), infected with C. difficile or pretreated with E. coli^{pHND19} or E. coli^{pAPyr} and then infected with C. difficile. Accordingly, E. coli^{pApyr} treatment improves the survival of mice infected with C. difficile. Log-rank (Mantel-Cox) test was used. *p<0.05, **p<0.01.

FIG. 17 shows for Example 4 the colon length in mice not treated (control), infected with C. difficile or pretreated with E. coli^{pHND19} or E. coli^{pApyr} and then infected with C. difficile. Accordingly, E. coli^{pApyr} treatment attenuates colitis induced by C. difficile infection. Means±SEM are shown. Two-tailed Mann-Whitney U-test was used. *p<0.05, **p<0.01.

FIG. 18 shows for Example 4 fecal Lipocalin-2 levels 72 h post infection in mice not treated (control), infected with C. difficile or pretreated with E. coli^{pHND19} or E. coli^{pAPyr} and then infected with C. difficile. Accordingly, E. coli^{pApyr} treatment attenuates intestinal inflammation induced by C. difficile infection. Means±SEM are shown. Two-tailed Mann-Whitney U-test was used. *p<0.05, **p<0.01.

FIG. 19 shows for Example 4 the serum Lipocalin-2 levels 72 h post infection in mice not treated (control), infected with C. difficile or pretreated with E. coli^{pHND19} or E. Coli^{pApyr} and then infected with C. difficile. Accordingly, E. coli^{pApyr} treatment attenuates the systemic inflammation induced by C. difficile infection. Means±SEM are shown. Two-tailed Mann-Whitney U-test was used. *p<0.05, **p<0.01.

FIG. 20 shows for Example 5 the treatment schedule in a mouse model of Clostridioides difficile infection after induction of dysbiosis with cefoperazone. Dysbiosis was induced by oral gavage of cefoperazone (2.5 mg/mouse) in the evening for 5 days. 10¹⁰ CFU of E. coli^{pHND19} or E. coli^{pApyr} were orally gavaged in the morning concomitantly to cefoperazone treatment. At day 6 the cefoperazone treatment was stopped and E. coli^{pHND19} or E. coli^{pApyr} gavaging was protracted for 3 additional days. Mice were then orally infected with 10⁵ C. difficile VPI 10463 spores. Mice were analysed 72 h post infection in order to evaluate intestinal inflammation.

FIG. 21 shows for Example 5 the survival rates of mice in the dysbiosis/Clostridioides difficile infection challenge model shown in FIG. 20. Prior to infection, mice were daily gavaged with 2.5 mg/mouse of cefoperazone in the evening and 10¹⁰ CFU of E. coli^{pHND19} or E. coli^{pApyr} in the morning. At day 6 the cefoperazone treatment was stopped and only bacterial treatment was performed for additional three consecutive days. Mice were then orally infected with 10⁵ C. difficile VPI 10463 spores. Percent survival of mice not treated (control), infected with C. difficile or pretreated with E. coli^{pHND19} or E. coli^{pApyr} and then infected with C. difficile. The figure shows that E. coli^{pApyr} treatment improves the survival in mice infected with C. difficile. Log-rank (Mantel-Cox) test was used. *p<0.05, ***p<0.001.

FIG. 22 shows for Example 5 the clinical score at 24 h post infection in mice not treated (control), infected with C. difficile or pretreated with E. coli^{pHND19} or E. coli^{pAPyr} and then infected with C. difficile. The data show that E. coli^{pApyr} treatment ameliorates the clinical score in mice infected with C. difficile. Means±SEM are shown. Two-tailed Mann-Whitney U-test was used. Two-tailed Mann-Whitney U-test * p<0.05, ** p<0.01, **** p<0.0001.

FIG. 23 shows for Example 6 the schedule for monocolonization of C57BL/6 GF mice with E. coli^{pBAD28} or E. coli^{pApyr}. Germ free mice were orally gavaged with 5×10⁹ CFU/mouse of E. coli^{pBAD28} or E. coli^{pApyr} and 28 days later small intestine epithelial cells were purified in order to perform transcriptomic analysis.

FIG. 24 shows for Example 6 the gene transcription in intestinal epithelial cells of monocolonized animals. The Volcano plot shows for each gene (dot) the differential expression [log₂ fold-change (log₂FC)] and its associated statistical significance (log₁₀ p-value) in gnotobiotic E. coli^{pApyr} vs E. coli^{pBAD28} WT mice. The dark gray dots indicate those genes with an FDR-corrected p-value<0.05 and |log₂FC|>1. The 79 down- and 53 up-regulated genes (FDR-corrected p-value<10⁻⁵ and |log₂FC|>1.5) are also highlighted by the two rectangles.

FIG. 25 shows for Example 6 the relative expression level (Z-score) of the differentially expressed genes by Gene Ontology (GO) analysis (FDR-corrected p-value<0.05 and log₂FC>1) in E. coli^{pApyr} vs E. coli^{pBAD28} monocolonized mice. z-score was calculated as the number of genes upregulated minus the number of genes downregulated divided for the square root of the total number of genes analyzed;

$$z = \frac{( n up - n down )}{\sqrt{n tot}} .$$

FIG. 26 shows for Example 6 Gene Ontology analysis of the differentially expressed genes (FDR-corrected p-value<0.05 and log₂FC>1) in E. coli^{pApyr} vs E. coli^{pBAD28} intestinal epithelial cells.

FIG. 27 shows for Example 7 blood glucose variation after 4 days of antibiotic treatment and 4 days of recovery (see diagram in FIG. 4) in control (PBS treated), antibiotic (ABX) treated, ABX+E. coli^{pHND19} and ABX+E. coli^{pAPyr} treated C57BL/6 mice. Means±SEM are shown. Two-tailed Mann-Whitney U-test was used. *p<0.05, **p<0.01.

FIG. 28 shows for Example 8 the experimental schedule in a mouse model of cefoperazone mediated dysbiosis and recovery protocol. Dysbiosis was induced by oral gavage of cefoperazone (2.5 mg/mouse) in the evening for 5 days. Concomitantly, 10¹⁰ CFU of E. coli^{pHND19} or E. coli^{pApyr} were orally gavaged in the morning. E. coli^{pHND19} and E. coli^{pApyr} treatments were protracted after cefoperazone treatment as indicated.

FIG. 29 shows for Example 8 the body weight variation at the end of the experiment in control (PBS treated), cefoperazone treated, cefoperazone+E. coli^{pHND19} and cefoperazone+E. coli^{pApyr} treated C57BL/6 mice. Means±SEM are shown. Two-tailed Mann-Whitney U-test was used. *p<0.05, **p<0.01.

FIG. 30 shows for Example 8 Percentage of white adipose tissue deposition normalized for the mouse body weight, in control (PBS treated), cefoperazone treated, cefoperazone+E. coli^{pHND19} and cefoperazone+E. coli^{pApyr} treated C57BL/6 mice. Means±SEM are shown. Two-tailed Mann-Whitney U-test was used. *p<0.05, **p<0.01.

FIG. 31 shows for Example 9 the experimental schedule in a mouse model of antibiotics-induced dysbiosis. Except for control mice, dysbiosis was induced by daily oral gavage of ABX for 4 consecutive days. On the same days as the antibiotic treatment, mice were orally gavaged with PBS or 40 μg of purified recombinant apyrase every 12 h.

FIG. 32 shows for Example 9 blood glucose variation in control (PBS treated), antibiotic (ABX) treated, ABX+apyrase treated C57BL/6 mice. Means±SEM are shown. Two-tailed Mann-Whitney U-test was used. **p<0.01.

FIG. 33 shows for Example 9 percent WAT deposition in control (PBS treated), antibiotic (ABX) treated, ABX+apyrase treated C57BL/6 mice. Means±SEM are shown. Two-tailed Mann-Whitney U-test was used. **p<0.01.

FIG. 34 shows for Example 6 the gene transcription in intestinal epithelial cells of monocolonized Igh-J^{-/-} animals. The Volcano plot shows for each gene (dot) the differential expression [log 2 fold-change (log 2FC)] and its associated statistical significance (logic p-value) in gnotobiotic E. coli^{pApyr} vs E. coli^{pBAD28} Igh-J^{-/-} mice. The two quadrants delineate the regions corresponding to FDR-corrected p value<10-5 and |log₂FC|>1.5 used to highlight most prominently regulated genes in the same experiment performed with WT mice shown in FIG. 24.

FIG. 35 shows for Example 6 body weight variation in wild-type C57BL/6 mice monocolonized with E. coli^{pApyr} or E. coli^{pBAD28}. Means±SEM are shown. Two-way ANOVA test was used. **p<0.01.

FIG. 36 shows for Example 6 fasting blood glucose measured in wild-type C57BL/6 GF mice or monocolonized with E. coli^{pApyr} or E. coli^{pBAD28}. Means±SEM are shown. Two-tailed Mann-Whitney U-test was used. **p<0.01, ***p<0.001.

FIG. 37 shows for Example 6 serum insulin quantification in wild-type C57BL/6 GF mice or monocolonized with E. coli^{pApyr} or E. coli^{pBAD28}. Means±SEM are shown. Two-tailed Mann-Whitney U-test was used. *p<0.05.

FIG. 38 shows for Example 6 the quantification of white adipose tissue (WAT) deposition in wild-type C57BL/6 GF mice or monocolonized with E. coli^{PAPYr} or E. coli^{pBAD28}. Means±SEM are shown. Two-tailed Mann-Whitney U-test was used. *p<0.05; **p<0.01.

FIG. 39 shows for Example 6 glucose homeostasis determined by glucose tolerance test (GTT) in wild-type C57BL/6 GF mice or monocolonized with E. coli^{pApyr} or E. coli^{pBAD28}. Means±SEM are shown. Two-way ANOVA was used. *p<0.05, **p<0.01, **** p<0.0001.

FIG. 40 shows for Example 6 body weight variation in C57Bl/6 Igh-J^{-/-} mice monocolonized with E. coli^{pApyr} or E. coli^{pBAD28}. Means±SEM are shown. Two-way ANOVA test did not reveal any statistically significant difference between the 2 groups.

FIG. 41 shows for Example 6 fasting blood glucose measured in C57Bl/6 Igh-J^{-/-} mice monocolonized with E. coli^{pApyr} or E. coli^{pBAD28}. Means±SEM are shown. Two-tailed Mann-Whitney U-test did not reveal any statistically significant difference between the 2 groups.

FIG. 42 shows for Example 6 glucose homeostasis determined by glucose tolerance test (GTT) in C57Bl/6 Igh-J^{-/-} mice monocolonized with E. coli^{pApyr} or E. coli^{pBAD28}. Means±SEM are shown. Two-way ANOVA test did not reveal any statistically significant difference between the 2 groups.

FIG. 43 shows for Example 7 WAT deposition normalized by total body weight in control, ABX, ABX+E. coli^{pHND19} and ABX+E. coli^{pApyr} treated C57BL/6 mice. Means±SEM are shown. Two-tailed Mann-Whitney U-test was used. *p<0.05, **p<0.01.

FIG. 44 shows for Example 7 blood glucose variation after 4 days of antibiotic treatment and 4 days of recovery in control, ABX, ABX+E. coli^{pHND19} and ABX+E. coli^{pApyr} C57BL/6 Igh-J^{-/-} mice. Means±SEM are shown. Two-tailed Mann-Whitney U-test was used. *p<0.05 FIG. 45 shows for Example 7 WAT deposition normalized by total body weight in control, ABX, ABX+E. coli^{pHND19} and ABX+E. Coli^{pApyr} C57BL/6 Igh-J^{-/-} mice. Means±SEM are shown. Two-tailed Mann-Whitney U-test was used. **p<0.01.

FIG. 46 shows for Example 10 caecum weight normalized by total body weight in control, ABX, ABX+E. coli^{pHND19} and ABX+E. coli^{pApyr} treated C57BL/6 wild-type mice. Means±SEM are shown. Two-tailed Mann-Whitney U-test was used. *p<0.05, **p<0.01.

FIG. 47 shows for Example 10 the colony forming units (CFU) of aerobic bacteria recovered from the MLN of control, ABX, ABX+E. coli^{pHND19} and ABX+E. coli^{pApyr} treated C57BL/6 wild-type mice. Dashed line indicates lower limit of detection. Means±SEM are shown. Two-tailed Mann-Whitney U-test was used. *p<0.05, **p<0.01.

FIG. 48 shows for Example 10 the CFU of anaerobic bacteria recovered from the MLN of control, ABX, ABX+E. coli^{pHND19} and ABX+E. coli^{pApyr} treated C57BL/6 wild-type mice. Dashed line indicates lower limit of detection. Means±SEM are shown. Two-tailed Mann-Whitney U-test was used. *p<0.05, **p<0.01.

FIG. 49 shows for Example 10 caecum weight normalized by total body weight in control, ABX, ABX+E. coli^{pHND19} and ABX+E. coli^{pApyr} treated C57BL/6 Igh-J^{-/-} mice. Means±SEM are shown. Two-tailed Mann-Whitney U-test was used. **p<0.01.

FIG. 50 shows for Example 10 the CFU of aerobic bacteria recovered from the MLN of control, ABX, ABX+E. coli^{pHND19} and ABX+E. coli^{pApyr} treated C57Bl/6 Igh-J^{-/-} mice. Dashed line indicates lower limit of detection. Means±SEM are shown. Two-tailed Mann-Whitney U-test was used. **p<0.01.

FIG. 51 shows for Example 10 the CFU of anaerobic bacteria recovered from the MLN of control, ABX, ABX+E. coli^{pHND19} and ABX+E. coli^{pApyr} treated C57Bl/6 Igh-J^{-/-} mice. Dashed line indicates lower limit of detection. Means±SEM are shown. Two-tailed Mann-Whitney U-test was used. *p<0.05, **p<0.01.

FIG. 52 shows for Example 11 the DNA fragment insertion for the integration of S. flexneri phoN2 gene in EcN genome. malP: EcN gene for maltodextrin phosphorylase; cat: E. coli gene for chloramphenicol acetyltransferase; phoN2: S. flexneri gene for apyrase; malT: EcN gene for the transcriptional activator of the maltose and maltodextrins operon; FRT: Flippase Recognition Target sequence; P_{cot}: promoter of the cat gene; P_{proD}: promoter of the phoN2 gene; BBa_BB0032 RBS: Ribosome Binding Site of the phoN2 gene; T_{phoN2}: transcriptional terminator of the phoN2 gene.

FIG. 53 shows for Example 11 the nucleotide sequence of the EcN malP gene portion (SEQ ID NO: 6). The malP stop codon is indicated in bold.

FIG. 54 shows for Example 11 the nucleotide sequence of the EcN malTgene portion (SEQ ID NO: 7). The malT start codon is indicated in bold.

FIG. 55 shows for Example 11 the nucleotide sequence (SEQ ID NO: 8) of the DNA fragment including the P_{proD} promoter, the BBa_BB0032 RBS, the S. flexneri phoN2 gene and the phoN2 transcriptional terminator. The P_{proD} sequence is underlined. The BBa_BB0032 RBS is shown in italics. The phoN2 start and stop codons are indicated in bold. The phoN2 transcriptional terminator is shown in bold italics.

FIG. 56 shows for Example 11 the nucleotide sequence of the DNA fragment including the E. coli cat gene flanked by the FRT sequences (SEQ ID NO: 9). The cat start and stop codons are indicated in bold. The FRT sequences are shown in italics.

FIG. 57 shows for Example 11 the malP-phoN2-malT recombinant genomic region of EcN::phoN2. malP: EcN gene for maltodextrin phosphorylase; phoN2: S. flexneri gene for apyrase; malT: EcN gene for the transcriptional activator of the maltose and maltodextrins operon; FRT: Flippase Recognition Target sequence; P_{proD}: promoter of the phoN2 gene; BBa_BB0032 RBS: Ribosome Binding Site of the phoN2 gene; T_{phoN2}: transcriptional terminator of the phoN2 gene.

FIG. 58 shows for Example 11 Apyrase detection in recombinant E. coli Nissle (EcN) EcN::phoN2 periplasmic extracts compared to non-recombinant E. coli Nissle (EcN) extracts. EcN and EcN::phoN2 clone 1 (cl 1) bacterial cultures were grown for 2.5 h, in LB medium, at 37° C. and harvested by centrifugation. The periplasmic fraction of each culture was isolated, precipitated with trichloroacetic acid (TCA), solubilized in Laemmli buffer and analyzed by Western blot using a polyclonal anti-apyrase rabbit serum.

FIG. 59 shows for Example 11 the dose-dependent degradation of ATP by EcN::phoN2 periplasmic extract. EcN and EcN::phoN2 clone 1 (cl 1) bacterial cultures were grown for 6 h, in LB medium, at 37° C. and harvested by centrifugation. The periplasmic fraction of each culture was isolated, dialyzed against PBS 1× and serially diluted with PBS 1×. The apyrase activity in periplasmic extracts (PE) was measured as percentage of degradation of 50 μM ATP relative to PBS 1×. Apyrase activity in PE was evaluated by an ATP-dependent bioluminescence assay with recombinant firefly luciferase and its substrate D-luciferin according to the manufacturer's protocol (Life Technologies Europe B.V.).

FIG. 60 shows for Example 12 the experimental layout showing the model of antibiotics-induced dysbiosis. 8-week old C57BL/6 male mice were randomly assigned to 4 different experimental groups: not treated (control), treated with antibiotics (ABX: Vancomycin 1.25 mg, ampicillin 2.5 mg and metronidazole 1.25 mg in 200 μl sterile water per mouse), treated with ABX and 10¹⁰ CFU of EcN and treated with ABX and 10¹⁰ CFU of EcN::phoN2.

FIG. 61 shows for Example 12 blood glucose variation after 4 days of antibiotic treatment and 4 days of recovery (see diagram in FIG. 60) in control, ABX, ABX+EcN or EcN::phoN2 treated C57BL/6 male mice. Means±SEM are shown. Two-tailed Mann-Whitney U-test was used. *p<0.05, **p<0.01.

FIG. 62 shows for Example 12 WAT deposition normalized by total body weight in control, ABX, ABX+EcN or EcN::phoN2 treated C57BL/6 male mice. Means±SEM are shown. Two-tailed Mann-Whitney U-test was used. *p<0.05, **p<0.01, ***p<0.001.

FIG. 63 shows for Example 13 the caecum weight normalized by total body weight in control, ABX, ABX+EcN or EcN::phoN2 treated C57BL/6 male mice. Means±SEM are shown. Two-tailed Mann-Whitney U-test was used. *p<0.05, **p<0.01, ***p<0.001, ****p<0.0001.

FIG. 64 shows for Example 13 CFU of aerobic bacteria recovered from the MLN of control, ABX, ABX+EcN or EcN::phoN2 treated C57BL/6 male mice. Dashed line indicates lower limit of detection. Means±SEM are shown. Two-tailed Mann-Whitney U-test was used. *p<0.05, **p<0.01.

FIG. 65 shows for Example 13 CFU anaerobic bacteria recovered from the MLN of control, ABX, ABX+EcN or EcN::phoN2 treated C57BL/6 male mice. Dashed line indicates lower limit of detection. Means±SEM are shown. Two-tailed Mann-Whitney U-test was used. *p<0.05, **p<0.01, ***p<0.001.

FIG. 66 shows for Example 14 the map of the pNZ-Apyr plasmid carrying the phoN2 gene encoding apyrase used to transform Lactococcus lactis. P_{nisA}, nisin A inducible promoter; SP usp45: signal sequence of usp45 gene; phoN2: S. flexneri apyrase gene; repC: replication gene C; repA: replication gene A; camR (cat): chloramphenicol resistance gene.

FIG. 67 shows for Example 15 a schematic of the components of each diet, expressed as percentages of total calories: normal diet (ND: 20% protein and 15% fat) and a modified diet able to induce dysbiosis (DID: 7% protein and 5% fat).

FIG. 68 shows for Example 15 the experimental layout for DID in 5 weeks old mice. At 5 weeks of age, female C67BL/6 mice were randomized into receiving either a normal diet (ND: 20% protein and 15% fat) or a modified diet able to induce dysbiosis (DID: 7% protein and 5% fat). During this period, DID mice were orally gavaged every day with PBS or 10¹⁰ of L. lactis^{pNZ} or L. lactis^{pNZ-Apyr}. After 8 weeks, mice were sacrificed and analyzed in order to evaluate apyrase effects on DID.

FIG. 69 shows for Example 15 the concentration of FITC in the serum assessed 4 h post dextran-FITC oral administration, after mice were fed the indicated diet for 8 weeks and daily gavaged with PBS or 10¹⁰ of L. lactis^{pNZ} or L. lactis^{pApyr} as indicated. Means±SEM are shown. Two-tailed Mann-Whitney U-test was used. *p<0.05, **p<0.01.

FIG. 70 shows for Example 15 CFU of aerobic bacteria recovered from the MLN of ND, DID, DID+L. lactis^{pNZ} and DID+L. lactis^{pNZ-Apyr} treated adult C57BL/6 mice. Dashed line indicates lower limit of detection. Means±SEM are shown. Two-tailed Mann-Whitney U-test was used. *p<0.05.

FIG. 71 shows for Example 15 CFU of anaerobic bacteria recovered from the MLN of ND, DID, DID+L. lactis^{pNZ} and DID+L. lactis^{pNZ-Apyr} treated adult C57BL/6 mice. Dashed line indicates lower limit of detection. Means±SEM are shown. Two-tailed Mann-Whitney U-test was used. *p<0.05.

FIG. 72 shows for Example 15 fecal LCN-2 concentration measured after mice were fed the indicated diet for 8 weeks and daily gavaged with PBS or 10¹⁰ of L. lactis^{pNZ} or L. lactis^{pApyr}. Means±SEM are shown. Two-tailed Mann-Whitney U-test was used. * p<0.05.

FIG. 73 shows for Example 16 the experimental layout for the neonatal model of DID. At eight weeks of age, female C57BL/6 mice were randomized into receiving either a normal diet (ND: 20% protein and 15% fat) or a modified diet able to induce dysbiosis (DID: 7% protein and 5% fat). After 15 days, ND and DID mice were mated with male mice. Starting immediately after birth, DID pups were orally gavaged with PBS or 10⁸ of L. lactis^{pNZ} or L. lactis^{pNZ-Apyr} two times a week until 21 days after birth. Pups were daily monitored for body weight, tail length and behavior.

FIG. 74 shows for Example 16 the concentration of FITC in the serum assessed 4 h post dextran-FITC oral administration in ND, DID, DID+L. lactis^{pNZ} and DID+L. lactis^{pNZ-Apyr} mice at 21 days after birth. Means±SEM are shown. Two-tailed Mann-Whitney U-test was used. *p<0.05, **p<0.01.

FIG. 75 shows for Example 16 CFU of aerobic bacteria recovered from the MLN of ND, DID, DID+L. lactis^{pNZ} and DID+L. lactis^{pNZ-Apyr} treated 21 days old C57BL/6 mice. Dashed line indicates lower limit of detection. Means±SEM are shown. Two-tailed Mann-Whitney U-test was used. *p<0.05, **p<0.01.

FIG. 76 shows for Example 16 CFU of anaerobic bacteria recovered from the MLN of ND, DID, DID+L. lactis^{pNZ} and DID+L. lactis^{pNZ-Apyr} treated 21 days old C57BL/6 mice. Dashed line indicates lower limit of detection. Means±SEM are shown. Two-tailed Mann-Whitney U-test was used. *p<0.05.

FIG. 77 shows for Example 17 body weight variation assessed at 21 days after birth in ND, DID, DID+L. lactis^{pNZ} and DID+L. lactis^{pNZ-Apyr} treated C57BL/6 mice. Means±SEM are shown. Two-tailed Mann-Whitney U-test was used. **p<0.01, ****p<0.0001.

FIG. 78 shows for Example 17 tail length measured at 21 days after birth in ND, DID, DID+L. lactis^{pNZ} and DID+L. lactis^{pNZ-Apyr} treated C57BL/6 mice. Means±SEM are shown. Two-tailed Mann-Whitney U-test was used. *p<0.05, ****p<0.0001.

FIG. 79 shows for Example 17 small intestine length measured at 21 days after birth in ND, DID, DID+L. lactis^{pNZ} and DID+L. lactis^{pNZ-Apyr} treated C57BL/6 mice. Means±SEM are shown. Two-tailed Mann-Whitney U-test was used. *p<0.05, ***p<0.001, ****p<0.0001.

FIG. 80 shows for Example 17 colon length measured at 21 days after birth in ND, DID, DID+L. lactis^{pNZ} and DID+L. lactis^{pNZ-Apyr} treated C57BL/6 mice. Means±SEM are shown. Two-tailed Mann-Whitney U-test was used. *p<0.05, **p<0.01, ****p<0.0001.

### EXAMPLES

In the following, particular examples illustrating various embodiments and aspects of the invention are presented. However, the present invention shall not to be limited in scope by the specific embodiments described herein. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become readily apparent to those skilled in the art from the foregoing description, accompanying figures and the examples below. All such modifications fall within the scope of the appended claims.

### Example 1: Design and Production of Apyrase-Expressing Bacteria

To obtain bacteria expressing apyrase, full length phoN2::HA fusion, encoding periplasmic ATP-diphosphohydrolase (apyrase) of Shigella flexneri (SEQ ID NO: 1) with a hemagglutinin (HA) fragment as tag were cloned into the polylinker site of plasmid pBAD28 (ATCC 8739387402), under the control of the PBAD L-arabinose inducible promoter. Thereby, plasmid pHND10 was generated, essentially as described in Santapaola, D., Del Chierico, F., Petrucca, A., Uzzau, S., Casalino, M., Colonna, B., Sessa, R., Berlutti, F., and Nicoletti, M. (2006). Apyrase, the product of the virulence plasmid-encoded phoN2 (apy) gene, is necessary for proper unipolar IcsA localization and for efficient intercellular spread. Journal of bacteriology 188, p. 1620-1627.

As control, plasmid pHND19 was produced essentially as described in Scribano, D., Petrucca, A., Pompili, M., Ambrosi, C., Bruni, E., Zagaglia, C., Prosseda, G., Nencioni, L., Casalino, M., Polticelli, F., et al. (2014). Polar localization of PhoN2, a periplasmic virulence-associated factor of Shigella flexneri, is required for proper IcsA exposition at the old bacterial pole. PloS one 9, e90230. In contrast to the pHND10 plasmid, the pHND19 plasmid (control) contains a phoN2_{R192P}::HA fusion, which encodes a loss-of-function isoform of apyrase carrying the R192P substitution.

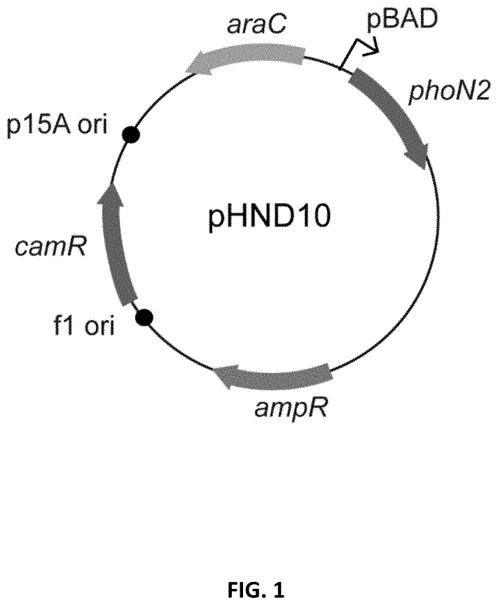

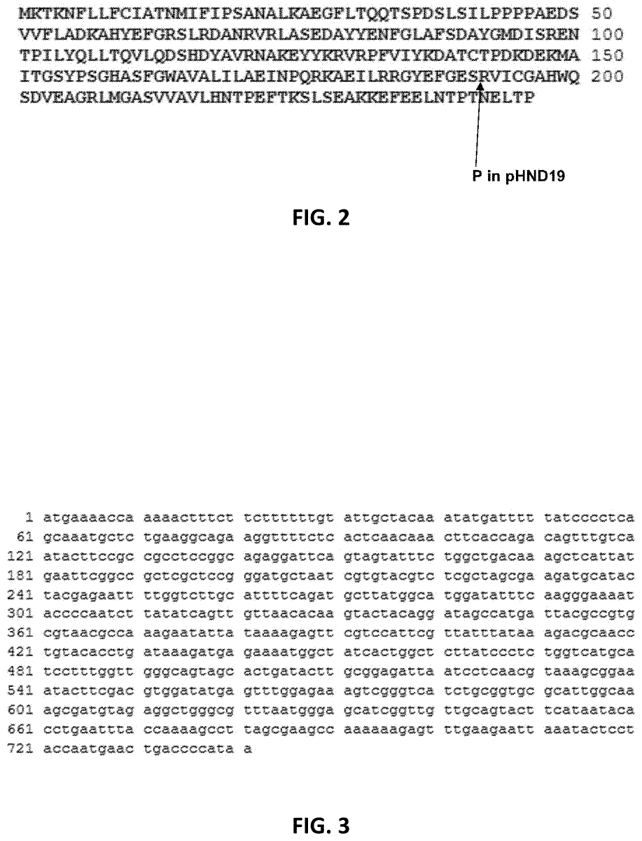

FIG. 1 shows a map of the pHND10 plasmid carrying the phoN2 gene encoding periplasmic ATP-diphosphohydrolase (apyrase). This map applies in general also to the pHND19 control plasmid, with the only difference that the loss-of-function isoform of apyrase carrying the R192P substitution is encoded instead of wild-type apyrase. FIG. 2 shows the amino acid sequence of wild-type phon2 protein (apyrase; SEQ ID NO: 1) and indicates the position of the R192P substitution in the loss-of-function isoform (SEQ ID NO: 2). The nucleotide sequence of the phoN2 gene used for generating pHND10 plasmid is shown in FIG. 3 (SEQ ID NO: 3).

Escherichia coli DH10B were transformed with pHND10 (E. coli^{pApyr}) or pHND19_{R192P} (E. coli^{pHND19}) and grown in LB medium supplemented with L-arabinose (0.03%) and ampicillin (100 μg/ml).

### Example 2: Administration of Bacteria Expressing Apyrase Reduces Dysbiosis-Induced Decreases in Microbiota Diversity

In order to investigate a possible beneficial effect of apyrase in the recovery from dysbiosis, a mouse model of induced dysbiosis was used. Dysbiosis was induced by daily oral gavage of a mix of antibiotics (ABX: Vancomycin 1.25 mg, ampicillin 2.5 mg and metronidazole 1.25 mg) for 4 days. After the antibiotic treatment, during the recovery phase, mice were orally gavaged for 4 days with PBS (control) or 10¹⁰ CFU (colony forming unit) of E. coli^{pApyr} or E. coli expressing the loss-of-function isoform of apyrase with the R192P amino acid substitution as described in Example 1 (E. coli^{pHND19})

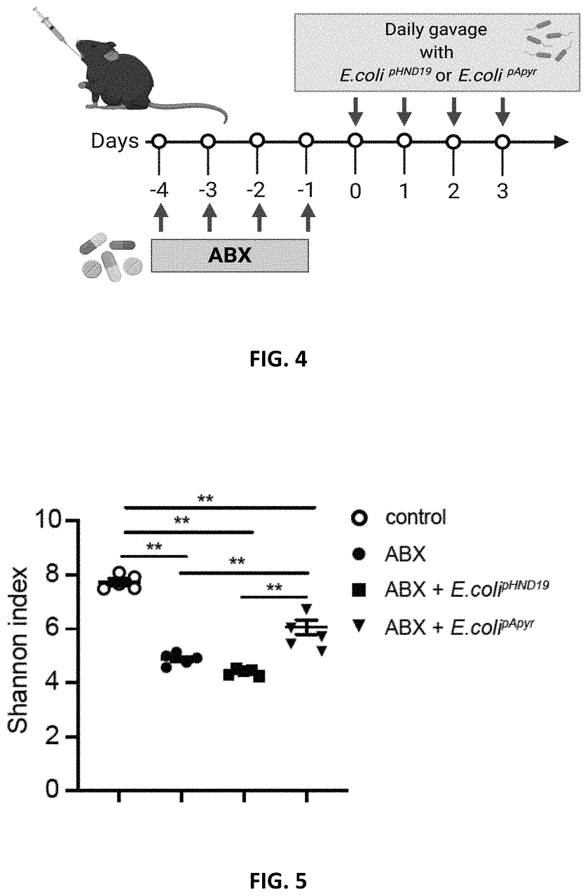

The treatment schedule is shown in FIG. 4. 8-week old C57BL/6 mice were randomly assigned to 4 different experimental groups: not treated (control), treated with antibiotics (ABX: Vancomycin 1.25 mg, ampicillin 2.5 mg and metronidazole 1.25 mg; in 200 μl sterile water per mouse), treated with ABX and 10¹⁰ CFU of E. coli^{pHND19}; and treated with ABX and 10¹⁰ CFU of E. coli^{pApyr}. At the end of the experiment, mice were sacrificed by CO₂ inhalation and caecal samples were collected.

Extraction, lysis and DNA isolation was done by using the Fast DNA Stool Mini Kit (Qiagen) according to manufacturer's recommendation. Bead beating was run on a fastprep24 instrument (MPBiomedicals; 4 cycles of 45 s at speed 4 followed) in 2 ml screwcap tubes containing 0.6 g 0.1 mm glass beads. 200 μl of raw extract was prepared for DNA-isolation. Concentration of the isolated DNA was assessed with PicoGreen measurement (Quant-iTT PicoGreenT dsDNA Assay Kit, Thermo Fisher) and integrity was checked by agarose gel electrophoresis for a random sample.

For the amplification of the bacterial 16S rRNA gene, a primer set specific for the V3-V4 hypervariable regions was used (Fw: 5′-CCT ACG GGN GGC WGC AG-3′ (SEQ ID NO: 4); and Rev: 5′-GAC TAC HVG GGT ATC TAA TCC-3′ (SEQ ID NO: 5)). Subsequently, the Illumina MiSeq platform and a v2 500 cycles kit were used to sequence the PCR libraries. The produced paired-end reads, which passed Illumina's chastity filter, were subjected to de-multiplexing and trimming of Illumina adaptor residuals using Illumina's real time analysis software included in the MiSeq reporter software v2.6 (no further refinement or selection). The quality of the reads was checked with the software FastQC version 0.11.8. The sequences were analyzed through the Qiime2 virtual environment (Bolyen E, Rideout J R, Dillon M R, et al. 2019. Reproducible, interactive, scalable and extensible microbiome data science using QIIME 2. Nature Biotechnology 37: 852-857. doi.org/10.1038/s41587-019-0209-9). The raw sequences were in total 4′896′770 (median=71942, mean=72′011.3, SD=15′891.2). The trimming step on the first 7 and the last 25 bases and the reads filtration have allowed to obtain excellent quality sequences (Phred>30). A denoising algorithm (DADA2 algorithm; Callahan B J, McMurdie P J, Rosen M J, Han A W, Johnson A J, Holmes S P. DADA2: High-resolution sample inference from Illumina amplicon data. Nat Methods. 2016; 13(7):581-583. doi:10.1038/nmeth.3869) was performed on these high quality sequences. The overlapping regions R1 and R2 were joined to obtain the non-chimeric reads used in the project. These were in total 1′145′671 (median=16′277, mean=16′848.1, SD=3′897.6).

The taxonomy assignment was performed by BLAST feature-classifier. It performs BLAST+ local alignment between query and reference reads. Then, it assigns consensus taxonomy to each query sequence on the last database version of Greengene (gg_12_10).

A rooted tree was constructed based on IQ-TREE stochastic algorithm that allows maximum likelihood analysis of large phylogenetic data (Nguyen L T, Schmidt H A, von Haeseler A, Minh B Q. IQ-TREE: a fast and effective stochastic algorithm for estimating maximum-likelihood phylogenies. Mol Biol Evol. 2015; 32(1):268-274. doi:10.1093/molbev/msu300).

Alpha diversity (Shannon-index; within-sample richness) was calculated using the main indexes to allow an exploration of data in term of richness and evenness. Alpha-diversity estimates were computed using the phyloseq R package (McMurdie P J, Holmes S. phyloseq: an R package for reproducible interactive analysis and graphics of microbiome census data. PLoS One. 2013; 8(4):e61217. Published 2013 Apr. 22. doi:10.1371/journal.pone.0061217). Statistically significant changes in the alpha diversity were determined through the Mann-Whitney signed-rank test.

Results are shown in FIG. 5. The metagenomic analysis revealed a strong reduction of alpha diversity, expressed as Shannon index, in control mice treated with ABX and ABX+E. coli^{pHND19}, indicating strong dysbiosis. However, treatment with E. coli^{pApyr} after dysbiosis induction resulted in a significant improvement of this parameter.

In order to determine similarity in bacterial composition in the different experimental groups, beta-diversity (between-sample dissimilarity) was analyzed in a principle coordinate analysis (PCoA) using a dissimilarity table obtained by Unweighted Unifrac algorithms (Lozupone C, Knight R. UniFrac: a new phylogenetic method for comparing microbial communities. Appl Environ Microbiol. 2005; 71(12):8228-8235. doi:10.1128/AEM.71.12.8228-8235.2005). Beta-diversity estimates were computed using the phyloseq R package (McMurdie P J, Holmes S. phyloseq: an R package for reproducible interactive analysis and graphics of microbiome census data. PLoS One. 2013; 8(4):e61217. Published 2013 Apr. 22. doi:10.1371/journal.pone.0061217). Permutational MANOVA (PERMANOVA) was performed on the unweighted UniFrac distance using the adonis( ) function of the vegan R package with 999 permutations.

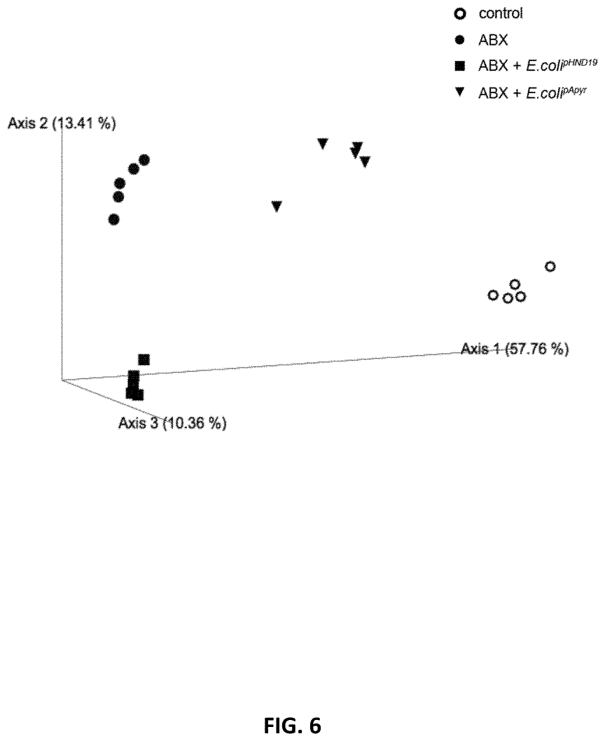

Results are shown in FIG. 6. Despite each antibiotic treatment group clustered separately from the untreated control (PERMANOVA<0.001), E. coli^{pAPyr} treated mice clustered closer to the control group, indicating an improved recovery of the physiological microbiota composition.

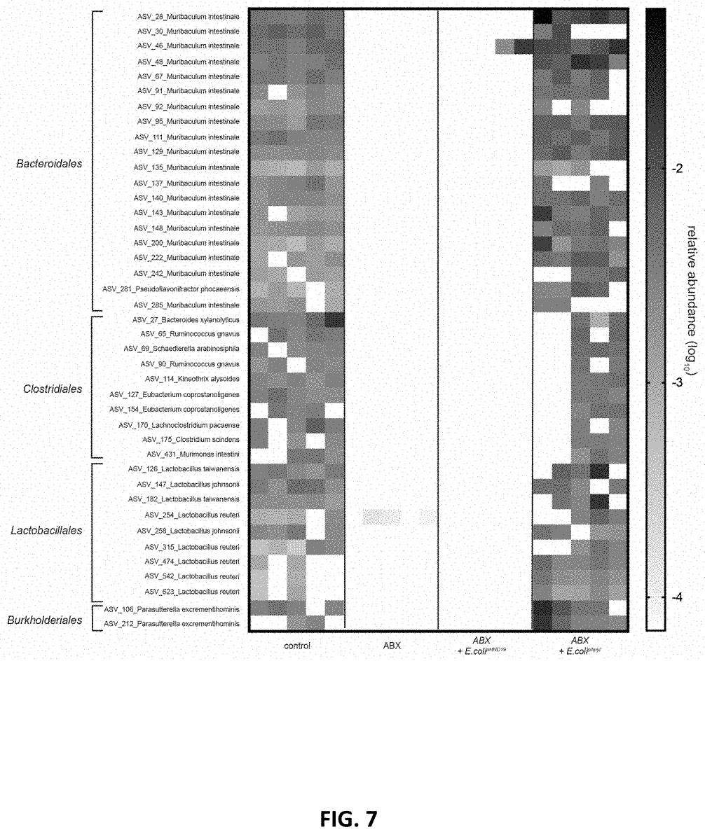

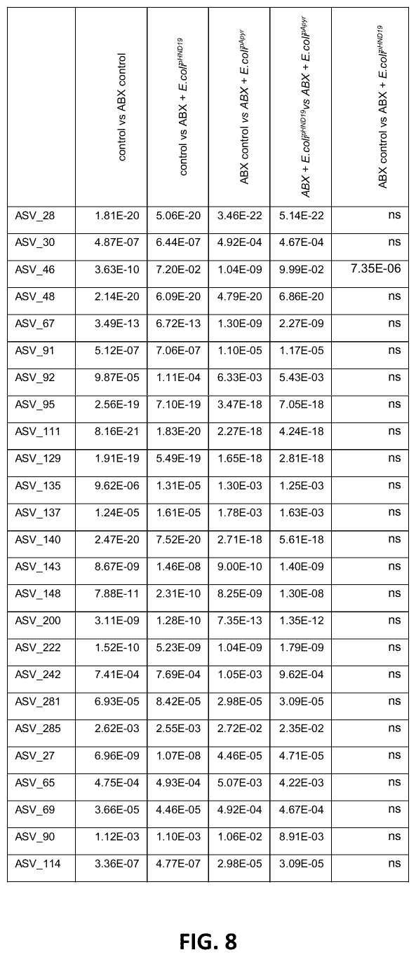

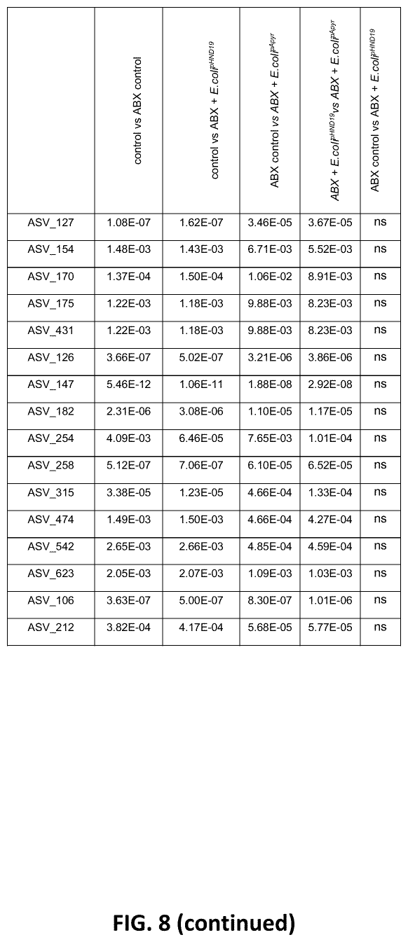

Differences in microbiota composition between all the populations were determined using Wald test using FDR p-value correction following DESeq2 read counts normalization (counts divided by sample-specific size factors determined by median ratio of gene counts relative to geometric mean per gene; Anders, S., Huber, W. Differential expression analysis for sequence count data. Genome Biol 11, R106 (2010). doi.org/10.1186/gb-2010-11-10-r106). Microbiota species were selected according to p<0.05 with Wald test using FDR p-value correction following DESeq2 read counts normalization. FIG. 7 shows a heatmap of differentially represented amplicon sequence variants (ASVs) that discriminate the caecal microbiota of the distinct experimental groups: control, ABX treated, ABX+E. coli^{pHND19} and ABX+E. coli^{pAPyr} treated C57BL/6 mice. FIG. 8 shows the p values of differentially represented ASVs, calculated with Wald test using FDR p-value correction following DESeq2 read counts normalization.

These results reveal that administration of apyrase expressing bacteria resulted in an improved microbial community structure with the selective preservation of 41 species belonging to the families of Bacteroidales, Clostridiales, Lactobacillales and Burkholderiales. Among Bacteroidales, Muribaculum intestinale was detected by multiple ASVs. The reduction of this bacterial species was shown to correlate with higher susceptibility to ileitis (Dobranowski, P. A., Tang, C., Sauve, J. P., Menzies, S. C., and Sly, L. M. (2019). Compositional changes to the ileal microbiome precede the onset of spontaneous ileitis in SHIP deficient mice. Gut Microbes 10, 578-598). E. coli^{pApyr} administration favored the preservation of Clostridium scindens, a bacterium that was shown to protect from C. difficile infection through the generation of secondary bile acids deoxycholic acid (DCA) and lithocholic acid (LCA). Reconstitution with C. scindens alone or within a bacterial consortium protected antibiotic treated mice from C. difficile intestinal colonization (Buffie, C. G., Bucci, V., Stein, R. R., McKenney, P. T., Ling, L., Gobourne, A., No, D., Liu, H., Kinnebrew, M., Viale, A., et al. (2015). Precision microbiome reconstitution restores bile acid mediated resistance to Clostridium difficile. Nature 517, 205-208). Different species belonging to Lactobacillales family were also significantly enriched in E. coli^{pApyr} treated mice. In particular, Lactobacillus johnsonii and Lactobacillus reuteri were significantly enriched. These two strains are commonly used as probiotics and were shown to confer protection against Citrobacter rodentium (Mackos, A. R., Eubank, T. D., Parry, N. M., and Bailey, M. T. (2013). Probiotic Lactobacillus reuteri attenuates the stressor-enhanced severity of Citrobacter rodentium infection. Infect Immun 81, 3253-3263) and Campylobacter jejuni (Bereswill, S., Ekmekciu, I., Escher, U., Fiebiger, U., Stingl, K., and Heimesaat, M. M. (2017). Lactobacillus johnsonii ameliorates intestinal, extra-intestinal and systemic pro-inflammatory immune responses following murine Campylobacter jejuni infection. Sci Rep 7, 2138) infection.

### Example 3: Administration of Bacteria Expressing Apyrase Reduces Effects of C. rodentium Infection after Induction of Dysbiosis

The gastrointestinal tract of mammals is colonized by hundreds of microbial species that confer colonization resistance against intestinal pathogens. Dysbiosis results in the loss of colonization resistance and susceptibility to enteric infections. Enterohemorrhagic Escherichia coli (EHEC), enteropathogenic E. coli (EPEC) and Citrobacter rodentium are Enterobacteriaceae that belong to the family of attaching and effacing (A/E) lesion-forming bacteria. EHEC and EPEC can cause severe intestinal inflammation and diarrhea. In addition, EHEC strains expressing the highly potent Shiga toxin (Stx) cause nephrotoxicity resulting in severe cases in the death of infected individuals (Collins, J. W., Keeney, K. M., Crepin, V. F., Rathinam, V. A., Fitzgerald, K. A., Finlay, B. B., and Frankel, G. (2014). Citrobacter rodentium: infection, inflammation and the microbiota. Nat Rev Microbiol 12, 612-623). Since human EHEC and EPEC only induce modest pathogenicity in antibiotic treated adult mice, C. rodentium is frequently used to mimic these infections in mice (Collins, J. W., Keeney, K. M., Crepin, V. F., Rathinam, V. A., Fitzgerald, K. A., Finlay, B. B., and Frankel, G. (2014). Citrobacter rodentium: infection, inflammation and the microbiota. Nat Rev Microbiol 12, 612-623; Bhinder, G., Sham, H. P., Chan, J. M., Morampudi, V., Jacobson, K., and Valiance, B. A. (2013). The Citrobacter rodentium mouse model: studying pathogen and host contributions to infectious colitis. J Vis Exp, e50222; Mallick, E. M., McBee, M. E., Vanguri, V. K., Melton-Celsa, A. R., Schlieper, K., Karalius, B. J., O'Brien, A. D., Butterton, J. R., Leong, J. M., and Schauer, D. B. (2012). A novel murine infection model for Shiga toxin-producing Escherichia coli. J Clin Invest 122, 4012-4024).

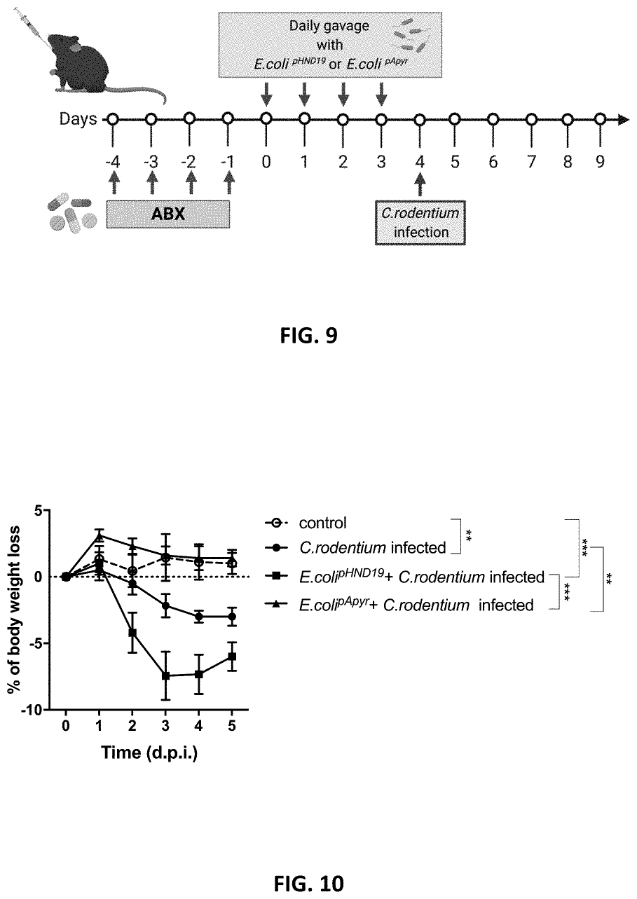

To investigate if the microbiota community structure induced by apyrase expressing bacteria could confer protection from C. rodentium infection, ABX was administered to C57BL/6 mice for 4 days, as described in Example 2. The treatment schedule is shown in FIG. 9. 8-week old C57BL/6 mice were randomly assigned to 4 different experimental groups: not treated (control), treated with antibiotics (ABX: Vancomycin 1.25 mg, ampicillin 2.5 mg and metronidazole 1.25 mg; in 200 μl sterile water per mouse), treated with ABX and 10¹⁰ CFU of E. coli^{pHND19}; and treated with ABX and 10¹⁰ CFU of E. coli^{pApyr}. After the antibiotic treatment, mice were orally gavaged for 4 days with PBS (control); or 10¹⁰ CFU of E. coli^{pHND19}; or E. coli^{pApyr}, similarly as in Example 2.

Thereafter, mice were orally infected with 10⁸ CFU/mouse of Citrobacter rodentium (except for the untreated control group). For infection experiments, Citrobacter rodentium ATCC®51459 (DBS100 strain) was cultured on LB agar plates and then expanded in Luria broth overnight at 37° C.

On days 0, 1, 2, 3, 4 and 5 post infection, the body weight of the animals was assessed. Results are shown in FIG. 10. Analysis of the percentage of body weight loss following C. rodentium infection revealed reduced body weight loss in mice orally gavaged with E. coli^{pAPyr} as compared to the groups treated with ABX alone or in combination with E. coli^{pHND19}, thus showing that administration of apyrase expressing bacteria improves mice resistance to C. rodentium infection.

In order to evaluate intestinal inflammation, mice were sacrificed 6 days post infection. Caecum was removed, opened longitudinally, delicately separated by caecal content and washed trice with ice cold PBS. The caecum was digested at 37° C. for 30 min with RPMI added with 5 mM EDTA for two times. The filtrated fragments were then digested in RPMI 5% FBS (fetal bovine serum), 1 mg/ml collagenase type II, 40 μg/ml DNase-I for 40 minutes. The filtered suspension, containing the caecum lamina propria cells, was centrifuged for 5 min at 1500 rpm and resuspended in RPMI complete medium. Single-cell suspensions from caecal lamina propria were stained with labelled antibodies diluted in PBS with 2% FBS for 20 min on ice. The following mouse antibodies (mAbs) were used for experiments: FITC conjugated anti mouse CD45 (1:200, clone: 30F11, Cat. #: 103108, Biolegend), PeCy7 conjugated anti-mouse CD11B (1:200, clone: N418, Cat. #:117318, Biolegend), biotinylated anti-mouse Ly6C (1:200, clone: HK1.4, Cat. #:128004, Biolegend), Pacific Blue conjugated anti-mouse Ly6G (1:200, clone: 1A8, Cat. #:127612 Biolegend), PE conjugated anti-mouse Gr1 (1:200, clone: RB6-8C5, Cat. #:50-5931-U100, TOMBO Bioscience), Streptavidin APC (1:200, Cat. #:405207, Biolegend). Samples were acquired on an LSR Fortessa (BD Biosciences, Franklin Lakes N.J., USA) flow cytometer. Data were analyzed using the FlowJo software (TreeStar, Ashland, Oreg., USA) or FACS Diva software (BD Biosciences, Franklin Lakes N.J., USA).

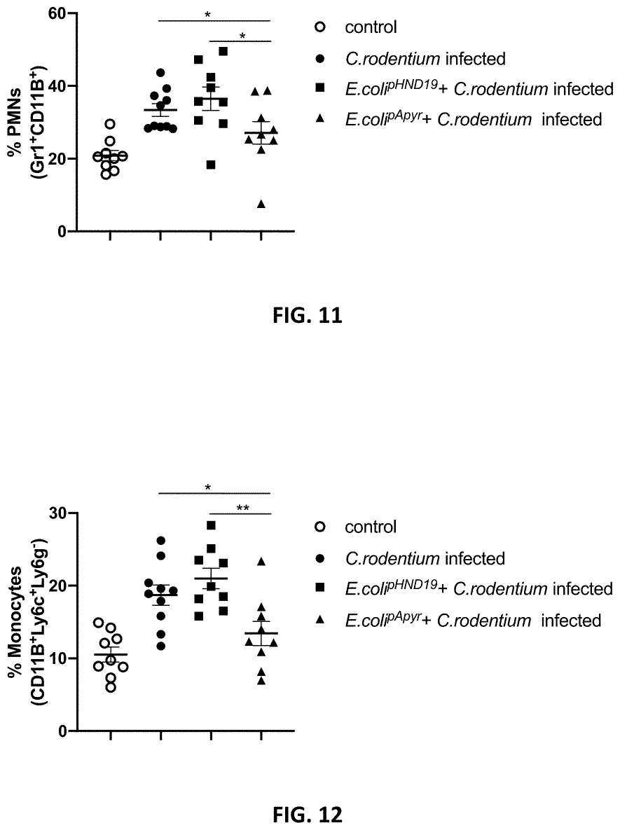

Statistical analysis of the frequencies of CD45⁺Gr1⁺CD11b⁺ polymorphonucleated (PMN) cells in caecum lamina propria of the different experimental groups was performed. Results are shown in FIG. 11. These data show a significant reduction of PMN cells in mice treated with ABX and gavaged with E. coli^{pApyr} as compared to the groups treated with ABX alone or ABX in combination with E. coli^{pHND19}. This result indicates that E. coli^{pApyr} administration attenuates dysbiosis-mediated infiltration of PMN in response to C. rodentium infection.

In addition, statistical analysis of the frequencies of CD45⁺CD11b⁺Ly6c⁺Ly6g⁻ inflammatory monocytes in caecum lamina propria of the different experimental groups was performed. Results are shown in FIG. 12. These data show a significant reduction of inflammatory monocytes in mice treated with ABX and gavaged with E. coli^{pApyr} as compared to the groups treated with ABX alone or ABX in combination with E. coli^{pHND19}. Analogously to the observation of reduced PMN infiltration, this result indicates that E. coli^{pApyr} administration attenuates dysbiosis-mediated inflammatory infiltration of intestinal lamina propria in response to C. rodentium infection.

### Example 4: Administration of Bacteria Expressing Apyrase Reduces Effects of C. difficile Infection after Induction of Dysbiosis

Clostridioides difficile is a major cause of antibiotic-associated diarrhea and has been shown to be associated with gut microbial dysbiosis, including reduced bacterial community diversity and depletion of key taxa.

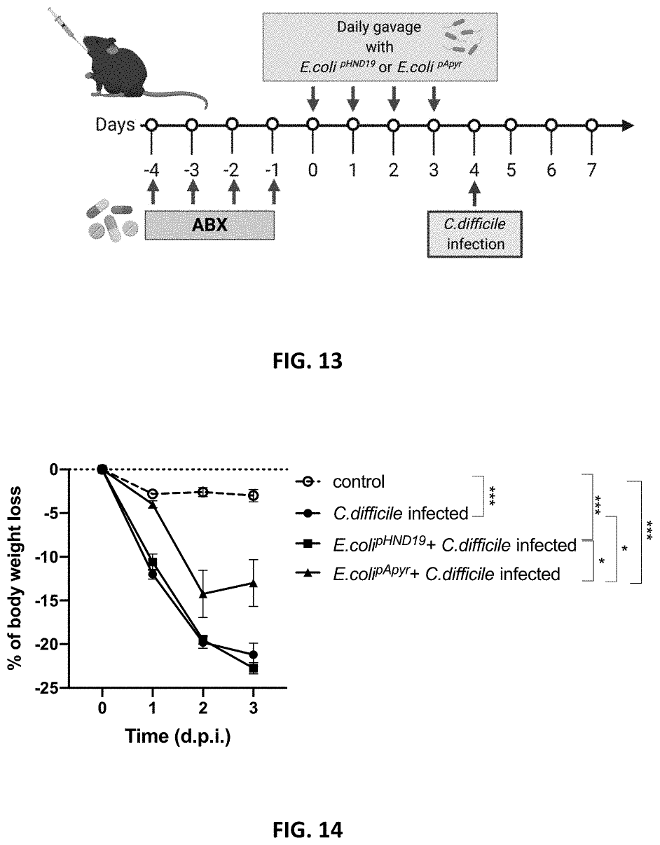

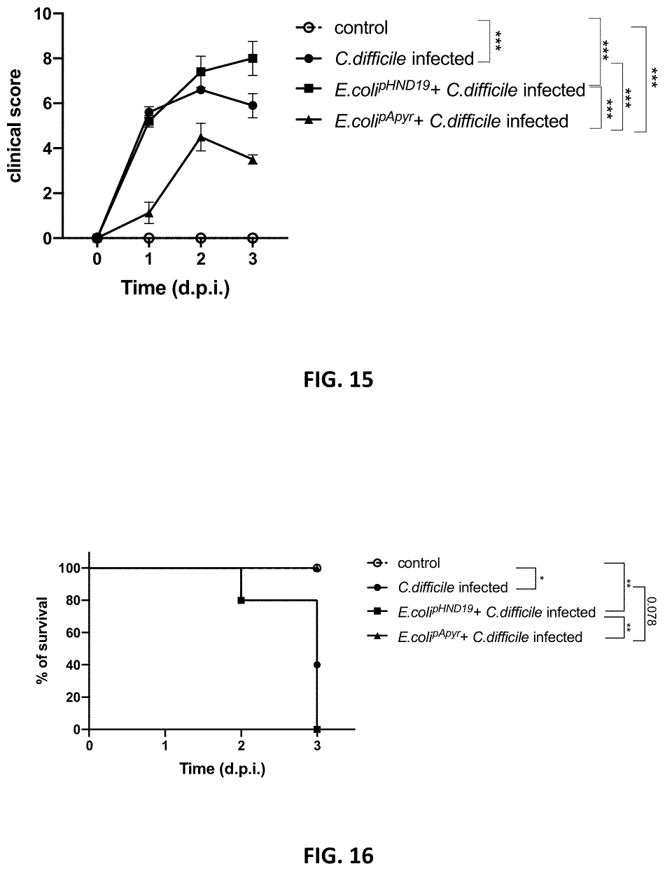

To investigate whether the microbiota community structure induced by apyrase expressing bacteria could counteract intestinal invasion by C. difficile, ABX was administered to C57BL/6 mice for 4 days to induce microbiota depletion and dysbiosis, as described in Examples 2 and 3. The treatment schedule is shown in FIG. 13. 8-week old C57BL/6 mice were randomly assigned to 4 different experimental groups: not treated (control), treated with antibiotics (ABX: Vancomycin 1.25 mg, ampicillin 2.5 mg and metronidazole 1.25 mg; in 200 μl sterile water per mouse), treated with ABX and 10¹⁰ CFU of E. coli^{pHND19}; and treated with ABX and 10¹⁰ CFU of E. coli^{pApyr}. After the antibiotic treatment, mice were orally gavaged for 4 days with PBS (control); or 10¹⁰ CFU of E. coli^{pHND19}; or E. coli^{pApyr}, similarly as in Examples 2 and 3.

Thereafter, mice were orally infected with 10 s C. difficile VPI 10463 spores (except for the untreated control group). To this end, Clostridioides difficile ATCC® 43255TM (VPI 10463 A⁺B⁺CDT⁻) spores were stocked at 10⁸/ml at −80° C. in PBS+1% BSA. Spores titres were confirmed by plating serial dilutions of the stocks on brain heart infusion (BD Biosciences) agar plates supplemented with 5 g/I yeast extract and 0.1% taurocholate to induce germination. Plates were kept at least 24 h in airtight canisters equipped with Oxoid Anaerogen™.

On days 0, 1, 2 and 3 post infection, the body weight, clinical scores and survival of the animals was assessed. The clinical score used in Clostridioides difficile infection is shown in Table 1:

TABLE 1
clinical score used in Clostridioides difficile infection.

| | | |
|---|---|---|
| body weight loss | from 0% to 5% | 0 |
| | from 6% to 10% | 1 |
| | from 11% to 15% | 2 |
| | more than 15% | 3 |
| dehydration | no | 0 |
| | mild | 1 |
| | severe | 2 |
| perianal appearance | clean | 0 |
| | lightly dirty | 1 |
| | wet tail | 2 |
| activity | normal | 0 |
| | reduced (hunched) | 1 |
| | absent (hunched) | 2 |

Results are shown in FIGS. 14 (body weight), 15 (clinical score) and 16 (survival). Analysis of the percentage of body weight loss following C. difficile infection revealed a significant reduction of body weight loss in mice orally gavaged with E. coli^{pApyr} as compared to the groups treated with ABX alone or in combination with E. coli^{pHND19}, thus indicating that administration of apyrase expressing bacteria protects mice from C. difficile infection. Evaluation of the clinical score over time during C. difficile infection confirmed that mice treated with ABX and gavaged with E. coli^{pApyr} were less affected by C. difficile infection compared to mice treated with ABX alone or ABX in combination with E. coli^{pHND19}. Analysis of survival in the different experimental groups revealed improved survival in mice treated with ABX and gavaged with E. coli^{pApyr} compared to mice treated with ABX alone or ABX in combination with E. coli^{pHND19}. This result indicates that E. coli^{pApyr} treatment profoundly attenuates the invasivity of C. difficile following antibiotic treatments.

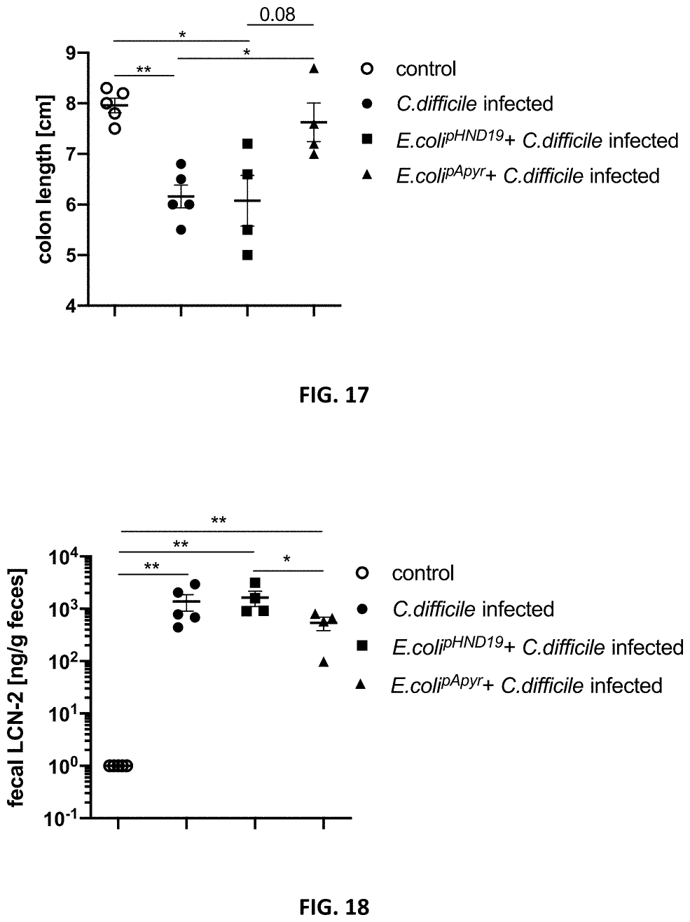

In order to evaluate intestinal inflammation, mice were sacrificed 72 h post infection and colon length was measured. Results are shown in FIG. 17. The measurement of colon length, an important parameter to score colitis, in mice treated with ABX and gavaged with E. coli^{pApyr} showed similar values to non-infected mice (control), whereas in mice treated with ABX alone or in combination with E. coli^{pHND19}, the colon length was drastically reduced. These data indicate that treatment with apyrase expressing bacteria attenuates colitis induced by C. difficile infection. C. difficile induced colitis was evaluated also by measuring fecal and serum lipocalin 2 (LCN-2), a marker of intestinal inflammation linked to epithelial damage and neutrophil infiltration, at 72 h post-infection, before sacrifice. The inflammation status of mice was evaluated by measuring the levels of Lipocalin-2 (LCN-2) in fecal supernatants via ELISA assay (R&D systems, DuoSet ELISA Mouse Lipocalin-2/NGAL). Briefly, feces were resuspended 0.01 g in 100 μl in PBS, centrifuged for 10 min at maximum speed and diluted before performing the ELISA assay, according to manufacturer's instructions. For the measurement of serum lipocalin 2 (LCN-2) levels, mice were bled and LCN-2 levels in the serum were assessed by ELISA assay as above.

Results for fecal LCN-2 levels are shown in FIG. 18. Mice treated with ABX and gavaged with E. coli^{pApyr} were characterized by lower levels of LCN-2 as compared to mice treated with ABX alone or ABX in combination with E. coli^{pHND19} further confirming that E. coli^{pApyr} can mitigate C. difficile mediated intestinal inflammation.

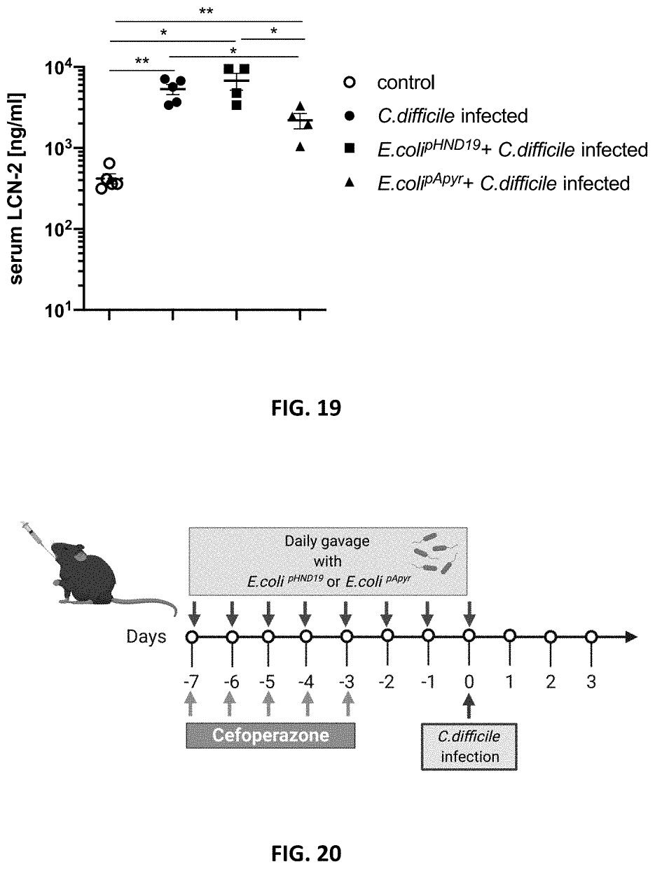

Results for serum LCN-2 levels are shown in FIG. 19. Quantification of serum LCN-2 in the different experimental groups revealed lower levels of LCN-2 in mice treated with ABX and gavaged with E. coli^{pApyr} as compared to mice treated with ABX alone or in combination with E. coli^{pHND19} This result indicates that E. coli^{pApyr} treatment limits the systemic dissemination of the pathogen.

### Example 5: Administration of Bacteria Expressing Apyrase Reduces Effects of C. difficile Infection after Induction of Dysbiosis in a Distinct Challenge Model

To address the possible effect of bacteria expressing apyrase also in distinct challenge model of C. difficile infection, dysbiosis was induced by daily gavaging the antibiotic cefoperazone in the evening (2.5 mg/mouse) for 5 consecutive days. On the same days, 10¹⁰ CFU of E. coli^{pHND19} or E. coli^{pApyr} were orally gavaged in the morning. At day 6 the cefoperazone treatment was stopped and E. coli^{pHND19} or E. coli^{pApyr} treatment was continued for three additional days. Mice were then orally infected with 10′ of C. difficile VPI 10463 spores essentially as described above (Example 4). The treatment schedule is shown in FIG. 20.

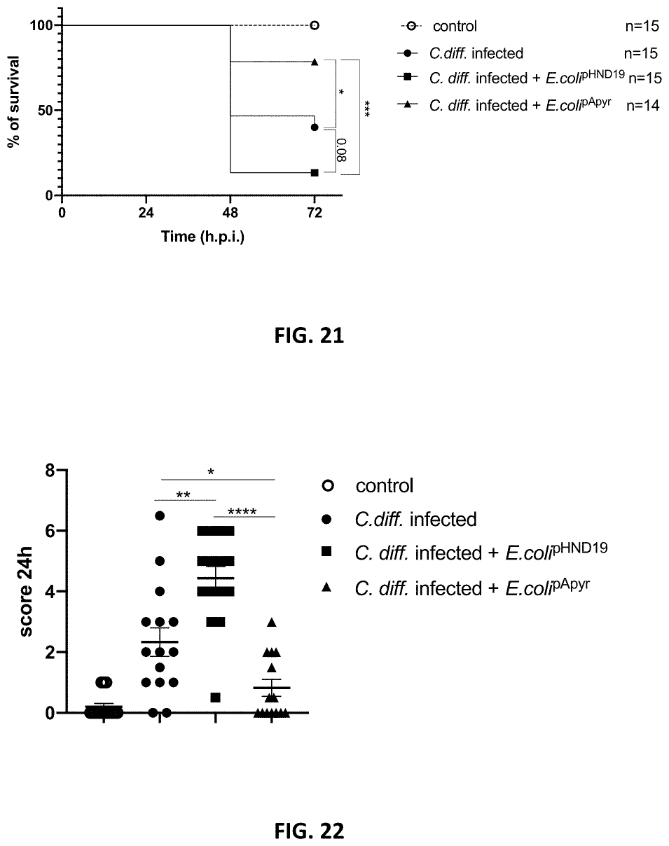

FIG. 21 shows the survival rates of mice treated as shown in FIG. 20. Analysis of mice survival in the different experimental groups revealed that oral gavaging with E. coli^{pApyr} resulted in the reduction of mortality compared to mice treated with cefoperazone alone or in combination with E. coli^{pHND19}. This result further supports the idea that administration of bacteria expressing apyrase effectively protects from C. difficile mediated mortality.

Clinical scores were assessed at 24 h post C. difficile infection as described above (Example 4, Table 1). Results are shown in FIG. 22. The analysis of clinical scores at 24 h post C. difficile infection showed that mice gavaged with E. coli^{pApyr} were characterized by less severe signs of infection compared to mice treated with cefoperazone alone or in combination with E. coli^{pHND19} Accordingly, administration of bacteria expressing apyrase ameliorates the clinical score in mice infected with C. difficile.

### Example 6: Monocolonization of Germ-Free Mice with Bacteria Expressing Apyrase

The transcriptional regulation in intestinal epithelial cells (IECs) plays a prominent role in the modulation of the composition of microbiota and host metabolic homeostasis, in a complex interplay between the immune system, the intestinal epithelium and the gut microbiota (Shulzhenko, N., Morgun, A., Hsiao, W., Battle, M., Yao, M., Gavrilova, O., Orandle, M., Mayer, L., Macpherson, A. J., McCoy, K. D., et al. (2011). Crosstalk between B lymphocytes, microbiota and the intestinal epithelium governs immunity versus metabolism in the gut. Nat Med 17, 1585-1593).

Germ-free (GF) mice monocolonized with E. coli^{pApyr} showed significantly reduced ATP in the intestine compared with mice monocolonized with bacteria carrying an empty vector (Perruzza, L., Gargari, G., Proietti, M., Fosso, B., D'Erchia, A. M., Faliti, C. E., Rezzonico-Jost, T., Scribano, D., Mauri, L., Colombo, D., et al. (2017). T Follicular Helper Cells Promote a Beneficial Gut Ecosystem for Host Metabolic Homeostasis by Sensing Microbiota-Derived Extracellular ATP. Cell Rep 18, 2566-2575). Consistent with a role of endoluminal ATP in regulating T follicular helper (Tfh) cell number and germinal center (GC) reaction in the Peyer's patches (PPs) of the small intestine (Proietti, M., Cornacchione, V., Rezzonico Jost, T., Romagnani, A., Faliti, C. E., Perruzza, L., Rigoni, R., Radaelli, E., Caprioli, F., Preziuso, S., et al. (2014). ATP-gated ionotropic P2X7 receptor controls follicular T helper cell numbers in Peyer's patches (PP) to promote host-microbiota mutualism. Immunity 41, 789-801), both Tfh and GC B cells were increased in animals colonized with apyrase-expressing bacteria. Moreover GF mice monocolonized with E. coli^{pApyr} showed higher amounts of E. coli specific IgA compared to E. coli^{pBAD28} monocolonized mice (Perruzza, L., Gargari, G., Proietti, M., Fosso, B., D'Erchia, A. M., Faliti, C. E., Rezzonico-Jost, T., Scribano, D., Mauri, L., Colombo, D., et al. (2017). T Follicular Helper Cells Promote a Beneficial Gut Ecosystem for Host Metabolic Homeostasis by Sensing Microbiota-Derived Extracellular ATP. Cell Rep 18, 2566-2575.). These data indicate that extracellular ATP released by commensal microbiota limits the secretory IgA response in the small intestine.

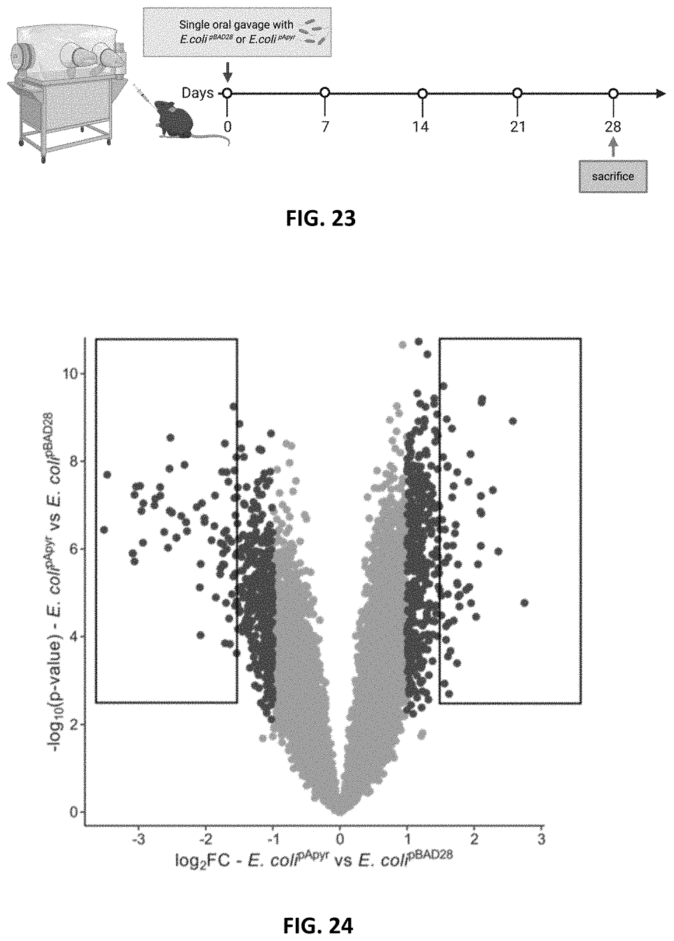

To investigate whether apyrase could affect host metabolism by regulating gene transcription in intestinal epithelial cells (IECs), monocolonized mice conditioned by apyrase were generated. To this end, germ-free (GF) mice were orally gavaged with E. coli^{pApyr} or E. Coli^{pBAD}28 once in order to generate monocolonized mice either conditioned by apyrase or not. Twenty-eight days later, mice were sacrificed and a genome-wide expression profiling was performed to compare ex vivo isolated IECs of differently colonized animals. The experimental schedule is shown in FIG. 23.

IECs were isolated by the method as described in Romagnani, A., Vettore, V., Rezzonico-Jost, T., Hampe, S., Rottoli, E., Nadolni, W., Perotti, M., Meier, M. A., Hermanns, C., Geiger, S., et al. (2017). TRPM7 kinase activity is essential for T cell colonization and alloreactivity in the gut. Nat Commun 8, 1917. Total RNA was extracted from IECs through Trizol precipitation (Invitrogen, Carlsbad, Calif.) and then digested with DNase I at 37° C. for 15 min to remove any contaminating DNA. The quality of total RNA was first assessed using an Agilent Bioanalyzer 2100 (Agilent Technologies, Palo Alto, Calif.). Biotin-labelled cDNA targets were synthesized starting from 150 ng of total RNA. Double stranded cDNA synthesis and related cRNA was performed with GeneChip® WT Plus Kit (Affymetrix, Santa Clara, Calif.). The sense strand cDNA was synthesized before with the same kit to be fragmented and labelled. All steps of the labelling protocol were performed as described by the kit's manufacturer, starting from 5.5 μg of ssDNA. Each eukaryotic GeneChip® probe array contained probe sets for several B. subtilis genes that were absent in the samples analyzed (lys, phe, thr, and dap). The GeneChip® Poly-A RNA Control Kit contained in vitro synthesized, polyadenylated transcripts for these B. subtilis genes that are pre-mixed at staggered concentrations to allow GeneChip® probe array users to assess the overall success of the assay. Poly-A RNA controls final concentration in each target were lys 1:100,000, phe 1:50,000, thr 1:25,000 and dap 1:6,667. Hybridization was performed using the GeneChip® Hybridization, Wash and Stain Kit containing a mix for target dilution, DMSO at a final concentration of 7% and pre-mixed biotin-labelled control oligo B2 and bioB, bioC, bioD and cre controls (Affymetrix cat #900299) at a final concentration of 50 pM, 1.5 pM, 5 pM, 25 pM and 100 pM, respectively. Fragmented and labelled sscDNA were diluted in hybridization buffer at a concentration of 23 ng/μl for a total of 2.3 μg and denatured at 99° C. for 5 min incubated at 45° C. for 5 min and centrifuged at maximum speed for 1 min prior to introduction into the GeneChip® cartridge. A single GeneChip® Mouse Clariom S was then hybridized with each biotin-labelled sense target. Hybridizations were performed for 16 h at 45° C. in a rotisserie oven. GeneChip® cartridges were washed and stained with GeneChip® Hybridization Wash and Stain Kit in the Affymetrix Fluidics Station 450 following the FS450_0007 standard protocol. The GeneChip® arrays were scanned using an Affymetrix GeneChip® Scanner 3000 7G using default parameters. Affymetrix GeneChip® Command Console software (AGCC) was used to acquire GeneChip® images and generate .DAT and .CEL files, which were used for subsequent analysis with proprietary software. Raw data was normalized using the quantile normalization of robust multiarray average (RMA) method. The identification of the differentially expressed transcripts and the hierarchical cluster analysis with Euclidean distance was performed using the commercial software Partek Genomics Suite (v6.6).

Results of the differential expression analysis are shown in FIG. 24. Differential expression analysis performed in gnotobiotic mice monocolonized with E. coli^{pApyr} or E. coli^{pBAD28} resulted in a transcriptional signature of 53 upregulated and 79 downregulated genes (highlighted by the rectangles in FIG. 24) in IECs isolated from E. coli^{pApyr} with respect to E. coli^{pBAD28} WT mice (FDR≤5% and absolute fold change≥1.5), suggesting that the absence of extracellular ATP (eATP) significantly influenced gene transcription in IECs.

Next, relative expression level (Z-score) of the differentially expressed genes were determined by Gene Ontology (GO) analysis. To this end, the list of differentially expressed genes were loaded into DAVID Bioinformatics Resources (v6.8; Huang da, W., Sherman, B. T., and Lempicki, R. A. (2009). Systematic and integrative analysis of large gene lists using DAVID bioinformatics resources. Nat Protoc 4, 44-57) for GO enrichment analysis and visualized using the R package GOplot Walter, W., Sanchez-Cabo, F., and Ricote, M. (2015). GOplot: an R package for visually combining expression data with functional analysis. Bioinformatics 31, 2912-2914) and gplots. Z-score was calculated as the number of genes upregulated minus the number of gene downregulated divided for the square root of the total number of genes analysed;

$$z = \frac{( n up - n down )}{\sqrt{n tot}} .$$

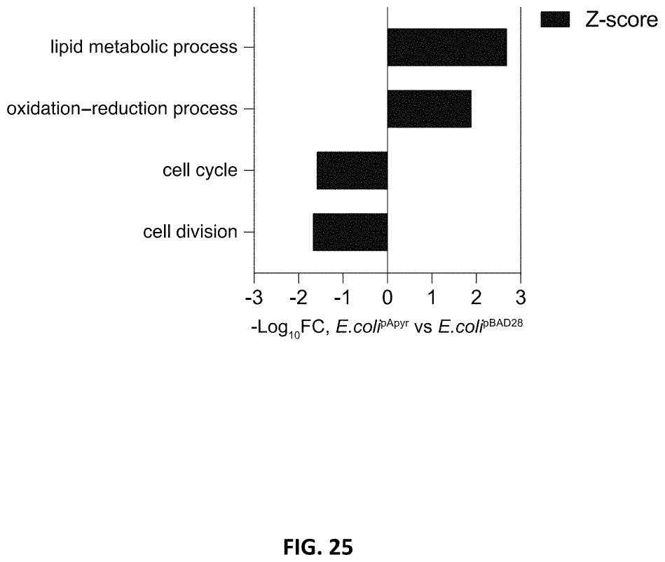

Results are shown in FIG. 25. Genes relative expression levels measured as Z-score, showed that E. coli^{pAPyr} treatment induced the downregulation of genes related to cell cycle and division and upregulation of genes related to lipid metabolic and oxidation-reduction processes. These two groups of genes are important for absorption of nutrients, and protection against chemical-induced oxidant injury. Moreover, upregulation of genes related to oxidation-reduction processes are important to preserve an environment that supports physiological processes and orchestrates networks of enzymatic reactions against oxidative stress (Circu, M. L., and Aw, T. Y. (2011). Redox biology of the intestine. Free Radic Res 45, 1245-1266).

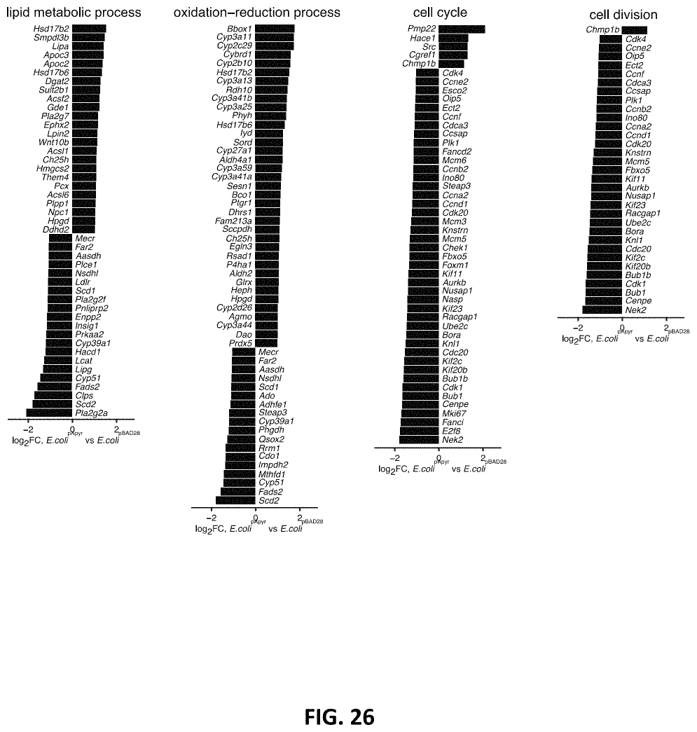

FIG. 26 shows the Gene Ontology analysis of differentially expressed genes in E. coli^{pApyr} vs E. coli^{pBAD28} intestinal epithelial cells of monocolonized mice. Functional over-representation analysis revealed that gene sets associated to DNA replication were enriched in IECs from E. Coli^{pBAD28} mice, whereas signature of metabolic functions governing principally lipids, fatty acids and vitamin A metabolism, solute carriers for carnitine, small peptides and ions were enriched in E. Coli^{pApyr} mice. E. Coli^{pApyr} monocolonized mice showed also an upregulation of genes belonging to the CYP family, that were shown to be important not only for drug metabolism but also in the oxidative, peroxidative and reductive metabolism of endogenous compounds such as steroids, bile acids, fatty acids, prostaglandins, biogenic amines and retinoids (Chang, G. W., and Kam, P. C. (1999). The physiological and pharmacological roles of cytochrome P450 isoenzymes. Anaesthesia 54, 42-50; Thelen, K., and Dressman, J. B. (2009). Cytochrome P450-mediated metabolism in the human gut wall. J Pharm Pharmacol 61, 541-558). These results suggest that depletion of eATP in the intestinal lumen modifies the interaction of colonizing bacteria with enterocytes, thereby conditioning their function and host metabolism.

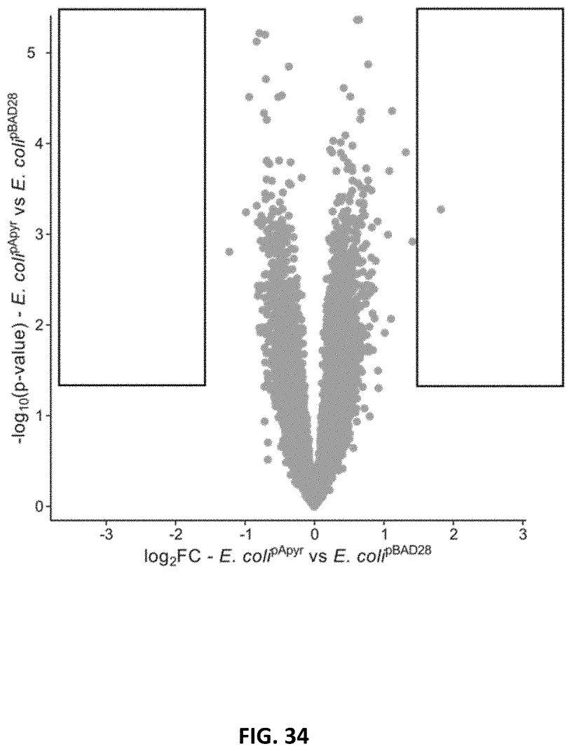

To evaluate the role of apyrase-mediated enhanced IgA generation on the observed conditioning of the transcriptional activity of the intestinal epithelium, genome wide transcription in the intestinal epithelium from monocolonized mice deficient in the generation of antibody response because of J segment deletion in Ig heavy chain locus (Igh-J^{-/-} mice) was analyzed. Strikingly, as shown by the volcano plot of FIG. 34, minimal differences in gene expression were detected between E. coli^{pApyr} and E. coli^{pBAD28} monocolonized Igh-J^{-/-} mice. These data indicate that enhanced IgA generation in wild-type mice monocolonized with E. coli^{pApyr} was responsible of favouring metabolic over immune functions in the enterocyte.

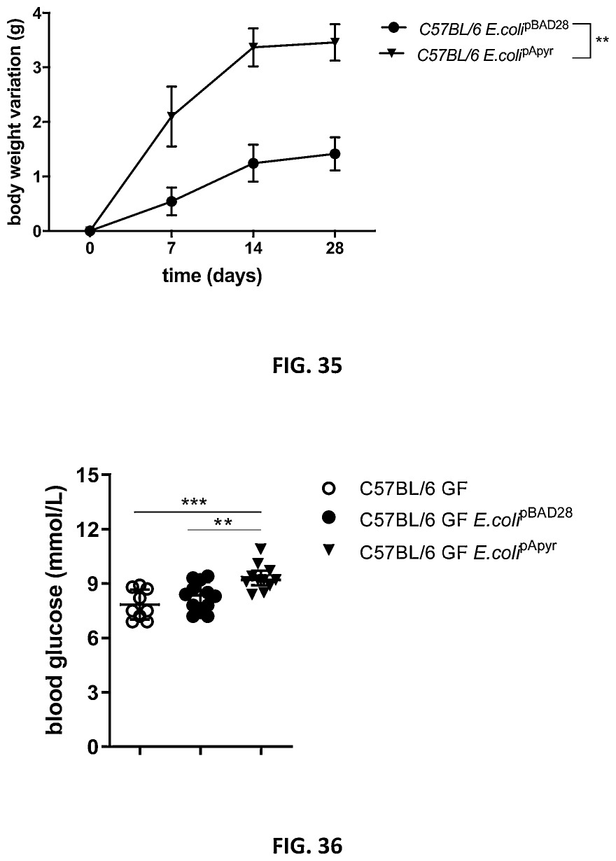

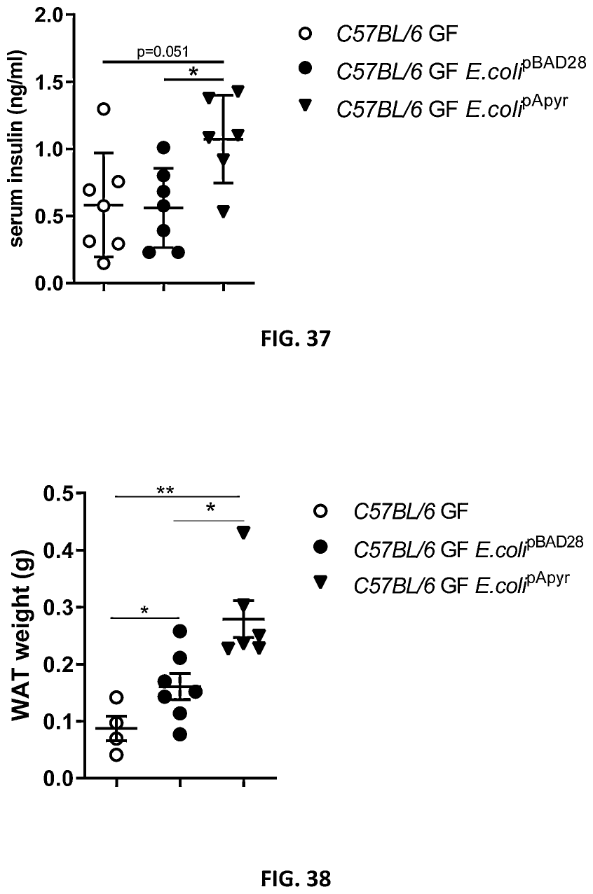

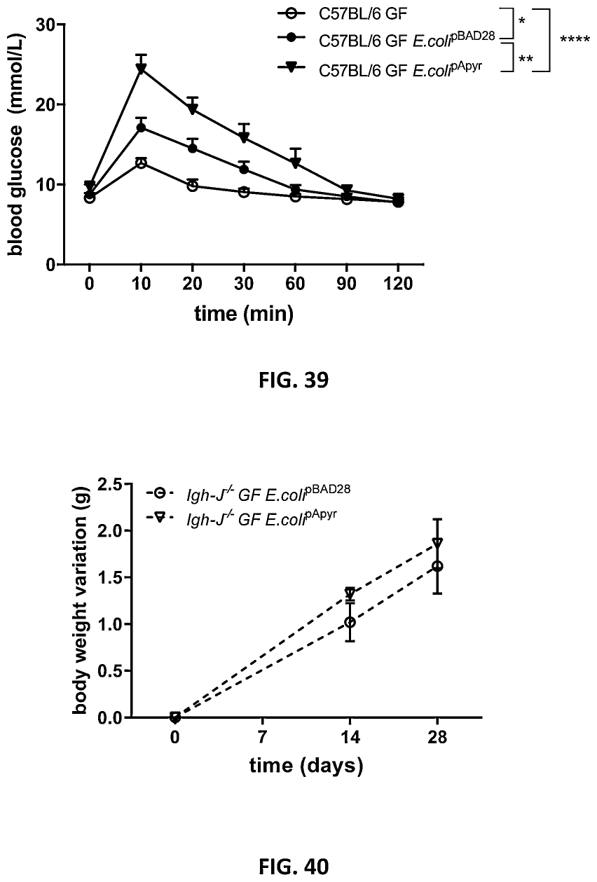

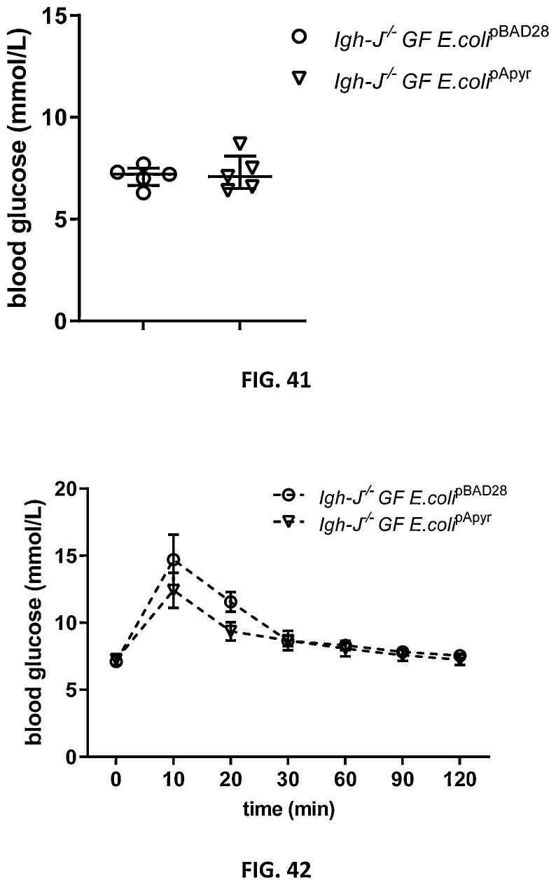

Accordingly, wild-type mice monocolonized with E. coli^{PAPYr} showed increased body weight variation (FIG. 35), blood glucose (FIG. 36), serum insulin (FIG. 37) and white adipose tissue (WAT) deposition (FIG. 38), as well as improvement of glucose tolerance test (FIG. 39) compared to mice monocolonised with E. coli^{pBAD28}. In contrast, no differences in body weight variation (FIG. 40), blood glucose (FIG. 41) and response in glucose tolerance test (FIG. 42) were observed in Igh-J^{-/-} mice monocolonized either with E. coli^{pBAD28} or E. Coli^{pApyr}. These data suggest that modulation of endoluminal ATP by apyrase can promote metabolic adaptation of the host via secretory IgA conditioning of commensal microbes.

### Example 7: Administration of Bacteria Expressing Apyrase Reduces Dysbiosis-Induced Hypoglycemia

To investigate the effects of apyrase on hypoglycemia due to antibiotics-mediated dysbiosis, dysbiosis was induced and bacteria expressing apyrase were administered as described in Example 2 (experimental schedule as shown in FIG. 4). Blood glucose was analyzed after 4 days of antibiotic treatment and 4 days of recovery (see FIG. 4).

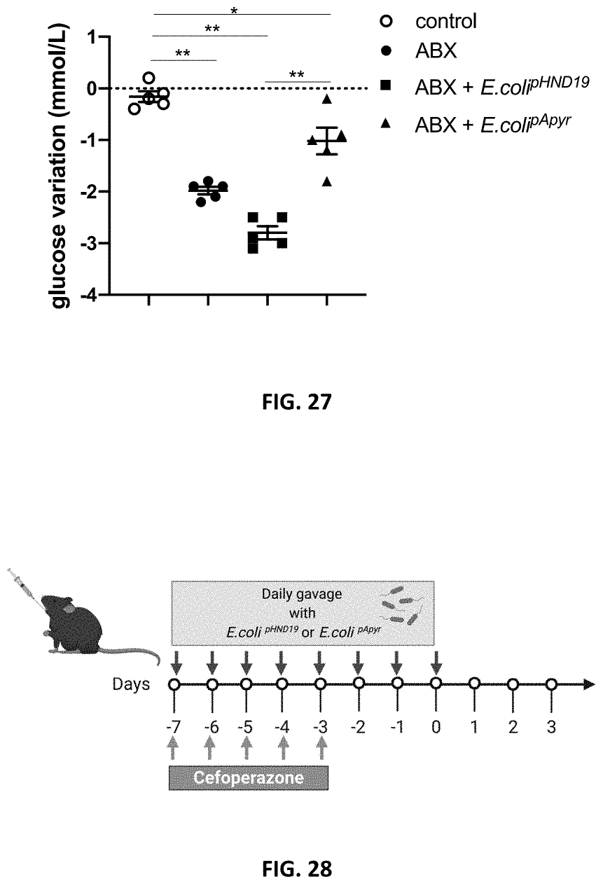

Results are shown in FIG. 27. Dysbiosis induced by antibiotics treatment resulted in a pronounced decrease in blood glucose (hypoglycemia). However, mice treated with apyrase expressing bacteria showed higher serum glucose levels as compared to ABX or ABX+E. coli^{pHND19} treated mice. These data indicate that E. coli^{pApyr} administration mitigates the antibiotic-mediated induction of hypoglycemia.

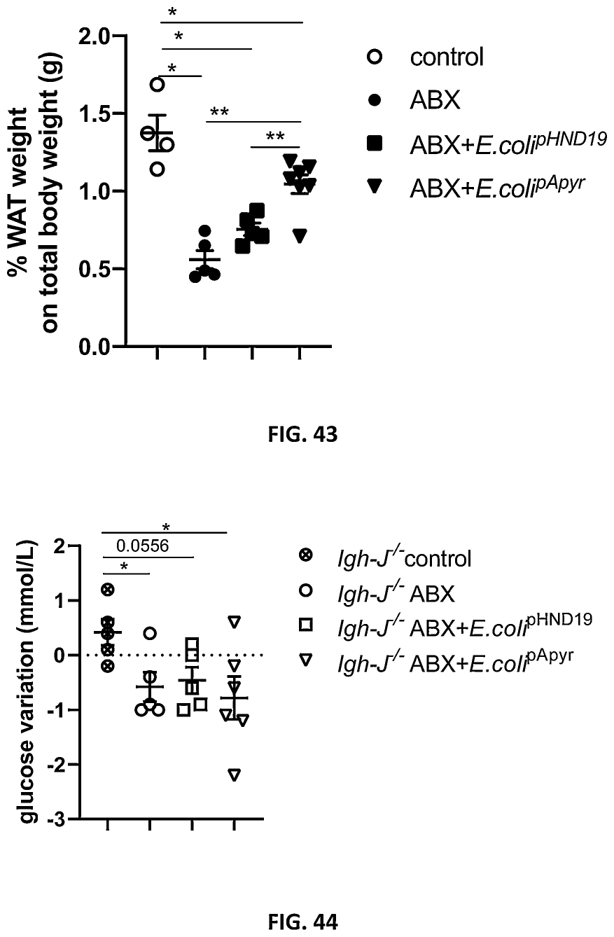

Moreover, white adipose tissue (WAT) was collected and quantified in order to evaluate the effect of apyrase on antibiotics-mediated WAT loss. Results are shown in FIG. 43. Quantification of WAT as percentage of total body weight revealed a significant reduction of WAT in ABX-induced dysbiosis (in both mice treated with ABX alone or in association with E. coli^{pHND19}) that was significantly attenuated by administration of E. coli^{PAPYr}.

No improvements in blood glucose levels (FIG. 44) and WAT deposition (FIG. 45) after ABX treatment were observed in Igh-J^{-/-} mice treated with E. Coli^{pApyr} as compared to the counterpart treated with ABX or the combination of ABX and E. coli^{pHND19}, consistent with the function of apyrase in conditioning host metabolism during antibiotic treatment via regulation of the secretory IgA response induced by commensal bacteria.

### Example 8: Mouse Model of Cefoperazone-Mediated Dysbiosis and Recovery

To further assess the efficacy of apyrase treatment in counteracting the metabolic impairment due to dysbiosis, dysbiosis was induced in a mouse model by daily administration of cefoperazone for five consecutive days. The experimental schedule is shown in FIG. 28.

To induce dysbiosis, mice were treated in the evening with cefoperazone (2.5 mg/mouse) for 5 consecutive days. Concomitantly, 10¹⁰ CFU of E. coli^{pHND19} or E. coli^{pApyr} were orally gavaged in the morning. At the end of cefoperazone treatment E. coli^{pHND19} or E. coli^{pApyr} administration was continued for 3 additional days.

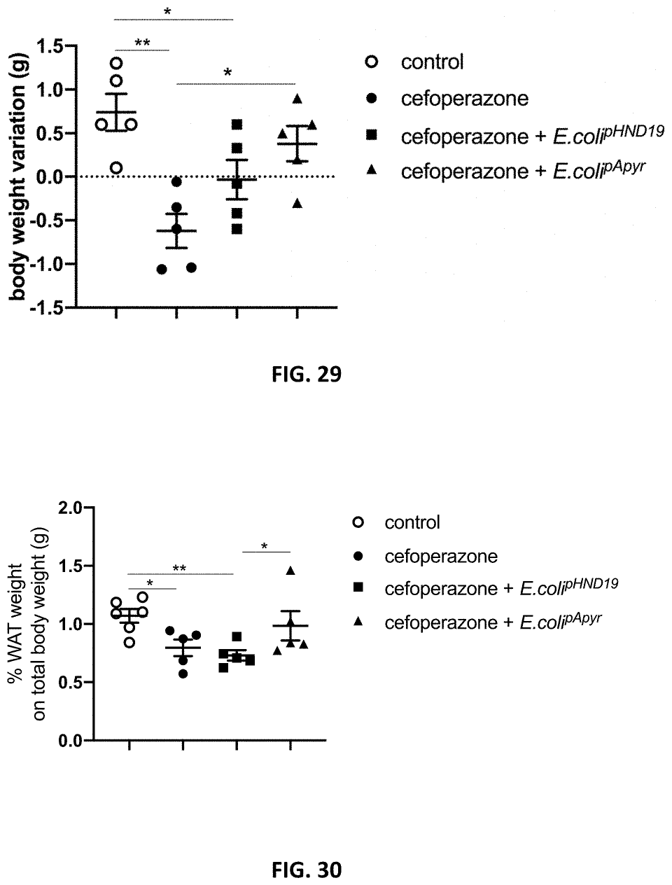

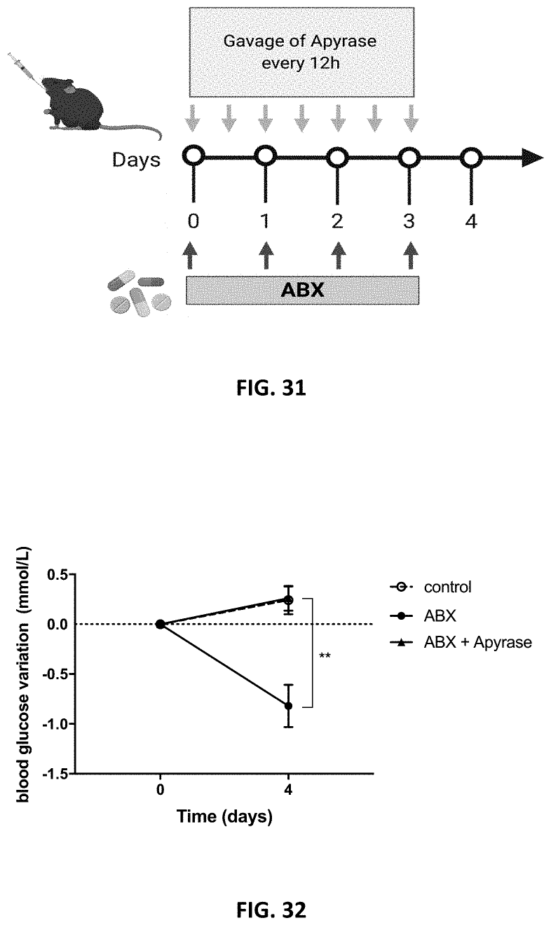

At the end of the experiment, the body weight of the animals was assessed. Results are shown in FIG. 29. The data reveal that cefoperazone-induced dysbiosis results in significantly decreased body weight (control mice compared to mice treated with cefoperazone alone or in association with E. coli^{pHND19}). In contrast, mice treated with cefoperazone and bacteria expressing apyrase (E. Coli^{PAPYr}) showed body weight gains that were not significantly different from untreated animals, while they were significantly higher with respect to mice treated with cefoperazone alone or in association with E. coli^{pHND19}. These data indicate that E. coli^{pApyr} administration attenuates body weight loss induced by cefoperazone mediated dysbiosis.

Mice were sacrificed at the end of the experiment and white perigonadal adipose tissue was collected and quantified. Results are shown in FIG. 30. Quantification of white adipose tissue (WAT) as percentage of total body weight revealed a significant reduction of WAT in cefoperazone-induced dysbiosis (control mice compared to mice treated with cefoperazone alone or in association with E. Coli^{pHND19}), while no significant variation was observed between control mice and mice treated with cefoperazone and bacteria expressing apyrase (E. Coli^{PAPYr}). Accordingly, E. coli^{pApyr} treatment attenuates WAT reduction induced by cefoperazone mediated dysbiosis.

### Example 9: Effects of Apyrase Administration in a Mouse Model of Antibiotics-Induced Dysbiosis

Next, the effects of administration of apyrase were investigated in a mouse model of antibiotics-induced dysbiosis. To this end, dysbiosis was induced by daily oral gavage of ABX for 4 consecutive days. Together with the antibiotic treatment, mice were orally gavaged with PBS or 40 μg of purified recombinant apyrase every 12 h.

At the end of the experiment, blood glucose levels were analyzed. Results are shown in FIG. 32. The analysis of blood glucose in mice orally gavaged with ABX and apyrase after four days of antibiotic treatment revealed values comparable to untreated mice and significantly higher blood glucose levels than mice treated with ABX without apyrase. Accordingly, the administration of the apyrase protein is sufficient to mitigate this metabolic modification in antibiotics-induced dysbiosis and apyrase attenuates the induction of hypoglycemia caused by antibiotic mediated dysbiosis.

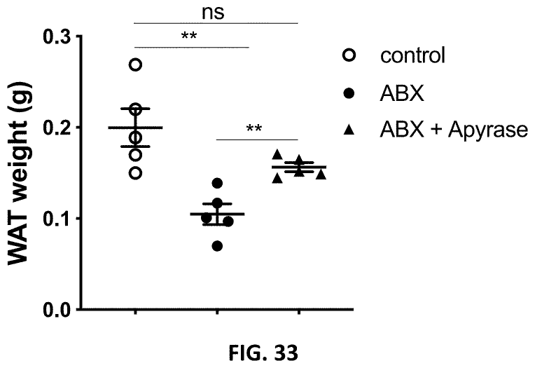

Mice were sacrificed at the end of the experiment and white perigonadal adipose tissue was collected and quantified. Results are shown in FIG. 33. Quantification of the WAT weight showed a significant decrease due to antibiotics-induced dysbiosis, while this effect was no longer observed in animals treated with apyrase, which show significantly higher WAT weight than animals suffering from antibiotics-induced dysbiosis without apyrase treatment. Accordingly, Apyrase treatment attenuates WAT reduction induced by antibiotics-induced dysbiosis.

### Example 10: Apyrase Attenuates Caecum Enlargement and Bacterial Translocation to the Mesenteric Lymph Node (MLN) Caused by Antibiotics-Mediated Dysbiosis

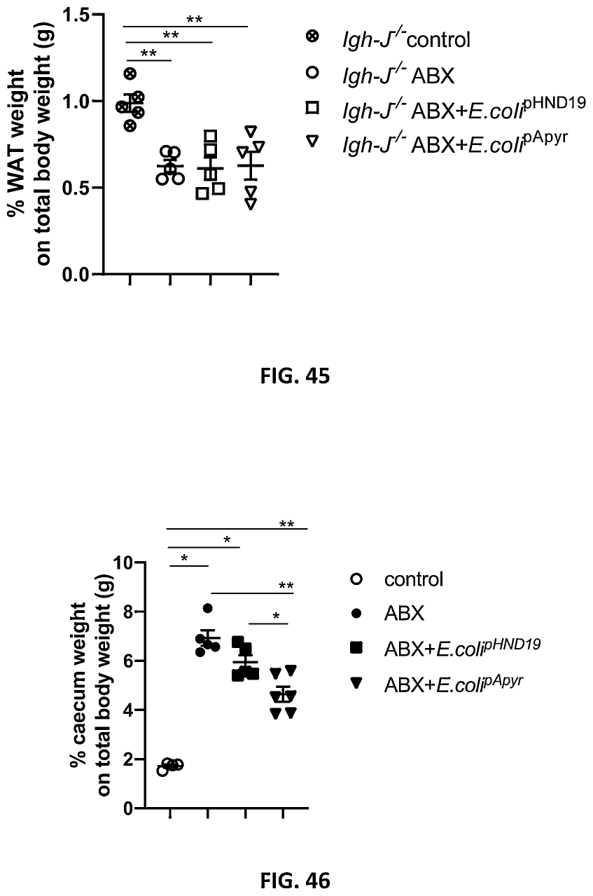

Intestinal dysbiosis caused by antibiotic treatment is characterized by a reduction in bacterial load and diversity, altered microbiota composition and impaired intestinal barrier integrity. Host features indicative of reduced bacterial load after antibiotic treatment could be examined by assessing cecum size, length and weight, which are reported to be grossly enlarged in germ-free animals (Devkota, S., Wang, Y., Musch, M. W., Leone, V., Fehlner-Peach, H., Nadimpalli, A., Antonopoulos, D. A., Jabri, B., and Chang, E. B. (2012). Dietary-fat-induced taurocholic acid promotes pathobiont expansion and colitis in Il10−/− mice. Nature 487, 104-108; Poteres, E., Hubert, N., Poludasu, S., Brigando, G., Moore, J., Keeler, K., Isabelli, A., Ibay, I. C. V., Alt, L., Pytynia, M., et al. (2020). Selective Regional Alteration of the Gut Microbiota by Diet and Antibiotics. Front Physiol 11, 797). To investigate the effect of apyrase on caecum enlargement caused by antibiotics-mediated dysbiosis, bacteria expressing apyrase were administered as described in Example 2 (experimental schedule as shown in FIG. 4). Caecum weight was analysed after 4 days of antibiotic treatment and 4 days of recovery (see FIG. 4). Results are shown in FIG. 46. Dysbiosis induced by antibiotics resulted in a pronounced increase in caecum weight. However, mice treated with E. coli^{pApyr} showed significantly reduced caecum weight as compared to mice treated with ABX or ABX and E. coli^{pHN19}. These data indicate that E. coli^{pApyr} administration mitigates antibiotics-induced caecum enlargement.

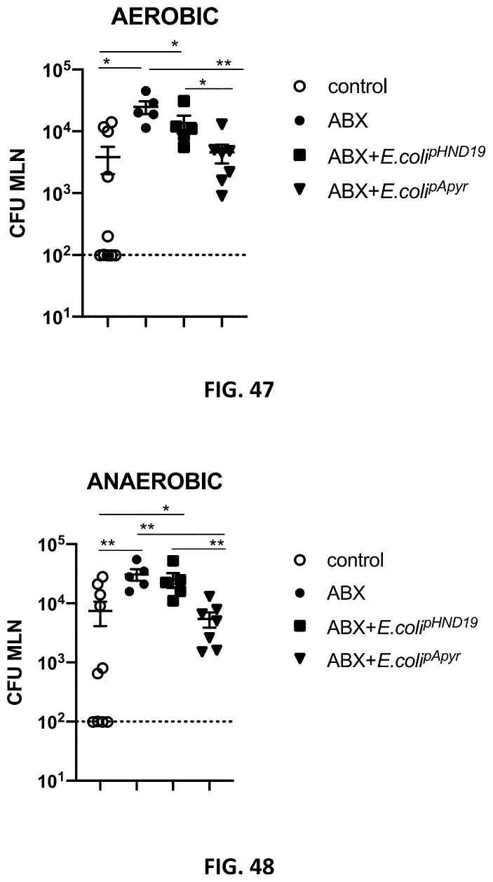

Antibiotic treatment induces impairment of gut barrier integrity and translocation of live commensal bacteria to the mesenteric lymph node (MLN) boosting an inflammatory response (Knoop K A, McDonald K G, Kulkarni D H, Newberry R D. Antibiotics promote inflammation through the translocation of native commensal colonic bacteria. (2016) Gut 65, 1100-9. doi: 10.1136/gutjnl-2014-309059). In order to investigate the effect of apyrase on antibiotics mediated bacterial translocation to the MLN, mice were sacrificed at the end of the experiment (see FIG. 4), MLN were harvested aseptically into RPMI and mechanically homogenized. Dilutions of homogenates were plated onto Schaedler agar (BD Biosciences). Plates were grown under aerobic or anaerobic culture conditions at 37° C. for 24-72 hours before enumeration of colonies. Results are shown in FIG. 47 and FIG. 48. Quantification of CFU from the MLN, both in aerobic and anaerobic conditions, revealed a significant increase in mice treated with ABX or ABX and E. coli^{pHN19} compared to untreated animals. However, mice treated with the combination of ABX and E. Coli^{pApyr} showed a number of CFU in the MLN that was not significantly different from untreated animals, while significantly lower with respect to mice treated with ABX alone or in association with E. coli^{pHN19}. These data indicate that E. coli^{pApyr} administration attenuates intestinal bacterial translocation induced by antibiotic-mediated dysbiosis.

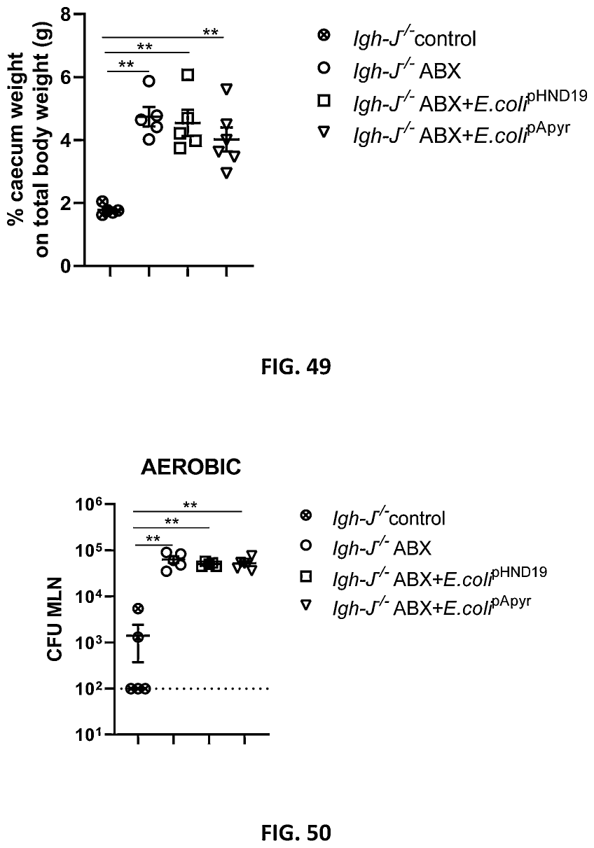

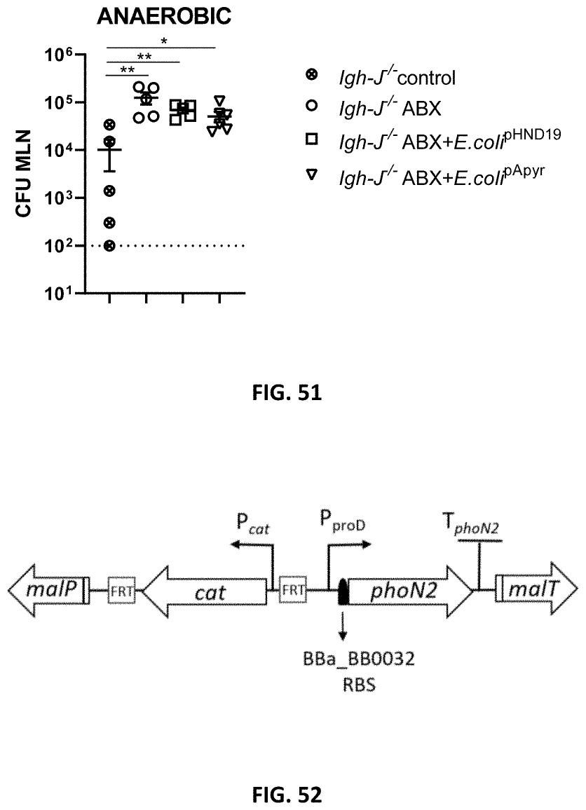

In order to understand whether secretory IgA were involved in apyrase mediated intestinal adaptation to dysbiosis by antibiotics, the same experiment as shown in FIG. 4 was performed using Igh-J^{-/-} mice. Caecum weight was analysed after 4 days of antibiotic treatment and 4 days of recovery. Results are shown in FIG. 49. Dysbiosis induced by antibiotics resulted in a pronounced increase in caecum weight in Igh-J^{-/-} mice treated with ABX, ABX+E. coli^{pHN19} and ABX+E. coli^{pApyr} compared to untreated animals. Quantification of CFU in the MLN of Igh-J^{-/-} mice, both in aerobic and anaerobic conditions, revealed the prominent translocation of bacteria in all the three different groups compared to the untreated group (FIG. 50 and FIG. 51). Strikingly, no amelioration of this feature was observed in animals treated with ABX in combination with E. coli^{pApyr} as compared to Igh-J^{-/-} mice treated with ABX or ABX+E. coli^{pHN19}. These data show the importance of secretory IgA elicited by Apyrase in controlling gut barrier impairment mediated by ABX treatment.

### Example 11: Generation of Recombinant Bacteria Heterologously Expressing Apyrase, which Carry the Apyrase Gene Integrated in their Genome (EcN::Phon2)

The apyrase expressing bacteria designed and produced as described in Example 1 above were obtained by transforming bacteria with plasmids encoding apyrase. Such plasmids may contain antibacterial resistance for the selection of the transformants. Such bacterial transformants typically bear multiple copies of the apyrase-encoding plasmid (and may be selected for antibiotic resistance). To investigate whether similar effects can be obtained in recombinant bacteria encoding apyrase in a heterologous manner in a single copy in their genome instead of multiple copies of extrachromosomal plasmids, bacteria having a single copy of the (heterologous) apyrase (phoN2) gene in the bacterial chromosome (non-transmissible) (without antibiotic resistance) were created.

To this end, the chromosomal integration of the Shigella flexneri phoN2 apyrase-encoding gene in the EcN genome (GenBank accession number CP007799.1) was performed by the λ Red recombineering approach (Datsenko K. A. and Wanner B. L. 2000 One-step inactivation of chromosomal genes in Escherichia coli K-12 using PCR products. Proc Natl Acad Sci USA. 97, 6640).

FIG. 52 schematically shows the DNA fragment used for the recombineering, including:
A portion of the EcN malP gene, coding for the maltodextrin phosphorylase enzyme;
The E. coli cat gene, which codes for the chloramphenicol acetyltransferase enzyme conferring resistance to the chloramphenicol antibiotic, flanked by the Flippase Recognition Target (FRT) sequences;
The Shigella flexneri phoN2 apyrase-encoding gene fused upstream with the P_{proD} synthetic promoter (Davis J. H., Rubin A. J. and Sauer R. T. 2011 Design, construction and characterization of a set of insulated bacterial promoters. Nucleic Acids Res. 9, 1131) and the BBa_BB0032 Ribosome Binding Site (RBS; iGEM Parts Registry), and downstream with the phoN2 transcriptional terminator;
A portion of the EcN malT gene, coding for the transcriptional activator of the maltose and maltodextrins operon.

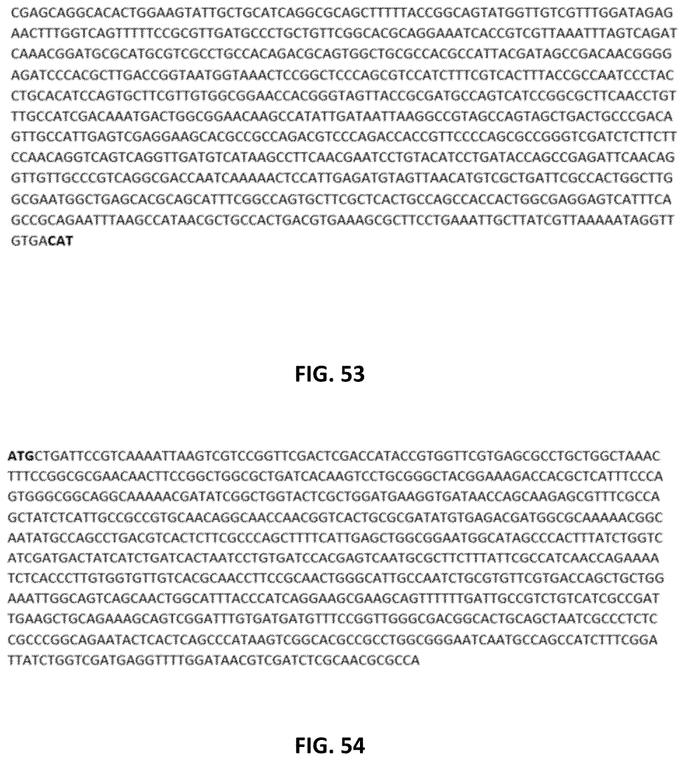

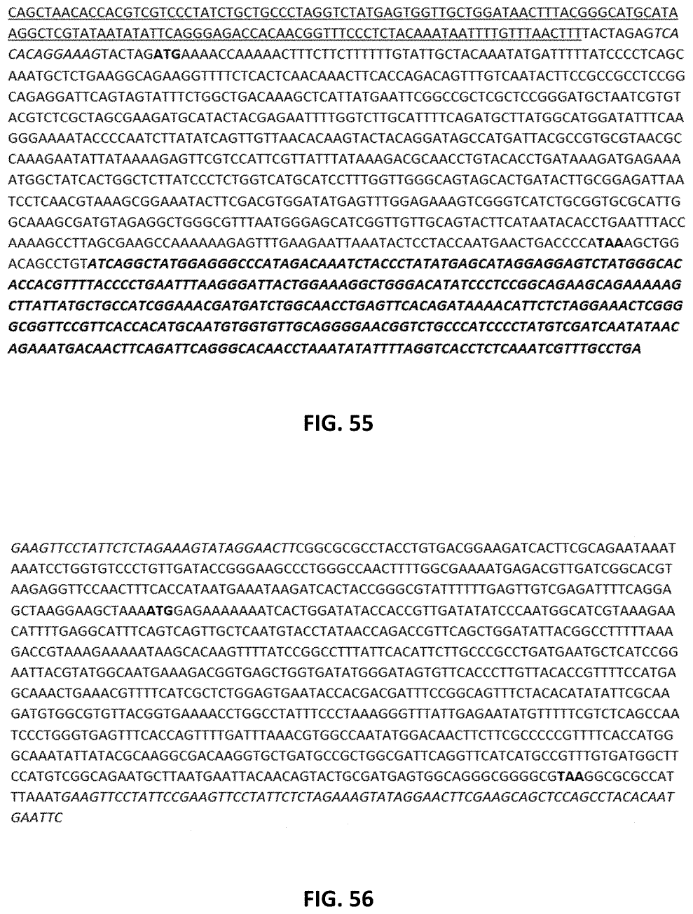

FIGS. 53 and 54 show the nucleotide sequences of EcN malP and malTgene portions, respectively (SEQ ID NOs 6 and 7, respectively). FIG. 55 shows the nucleotide sequence of the DNA fragment, including the P_{proD} promoter, the BBa_BB0032 RBS, the S. flexneri phoN2 gene and the phoN2 transcriptional terminator (SEQ ID NO: 8). FIG. 56 shows the nucleotide sequence of the DNA fragment, including the E. coli cat gene flanked by the FRT sequences (SEQ ID NO: 9).

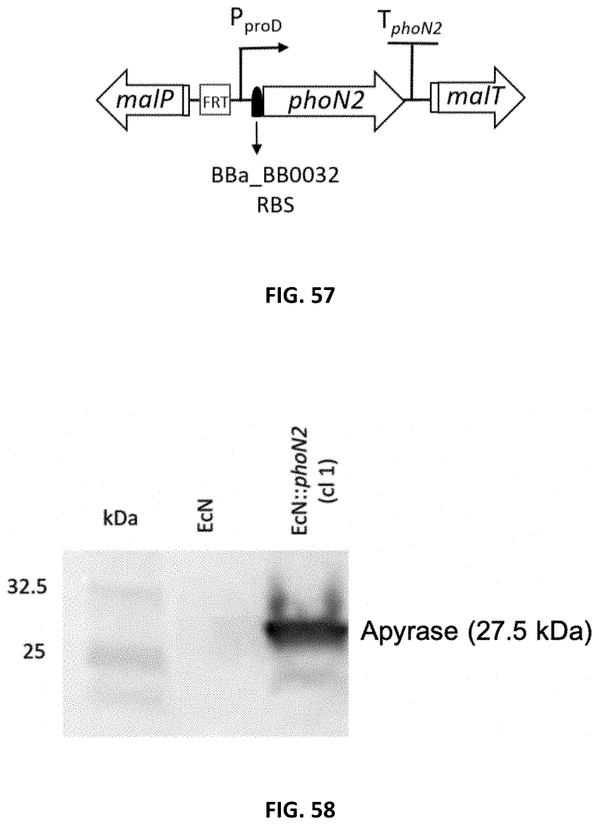

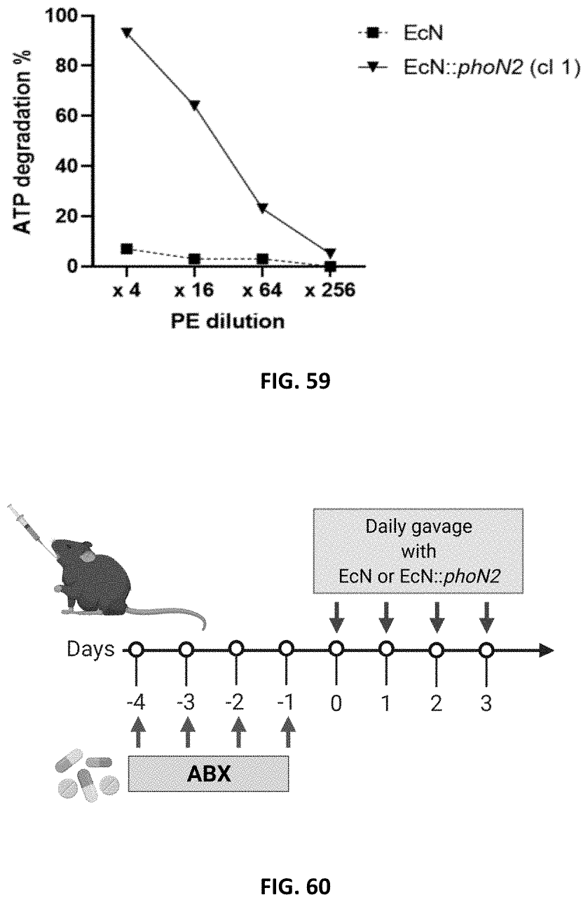

To perform the recombineering in the EcN genome, the insertion DNA fragment was transformed in an EcN strain carrying the pKD46 plasmid, which expresses the phage λ Red recombinase. The λ Red-mediated homology recombination at the malP and malT sites promoted the integration of the insertion DNA fragment in the malP-malT intergenic region of EcN. After pKD46 removal, the EcN clones carrying the insertion DNA fragment in the genome were selected for chloramphenicol resistance and checked by PCR for the correct integration in the genome. The EcN clones selected for the correct integration of the insertion DNA fragment were transformed with the pCP20 plasmid, which expresses the yeast Flp recombinase (Flippase), to excise the chloramphenicol resistance cassette from the genome. After pCP20 removal, the EcN recombinant clones not carrying the chloramphenicol cassette in the genome were selected for chloramphenicol sensitivity and checked by PCR for the correct excision of the cassette from the genome. The resulting EcN recombinant clones carrying the S. flexneri phoN2 gene in the malP-malT intergenic region were named EcN::phoN2. FIG. 57 schematically shows the malP-phoN2-malT recombinant genomic region of the obtained EcN::phoN2 clones. FIG. 58 shows the expression of apyrase in one selected EcN::phoN2 clone (cl 1) in a Western-Blot of periplasmic extracts. In addition, the activity of the enzyme in EcN::phoN2 cl 1 was verified. FIG. 59 shows the dose-dependent degradation of ATP by EcN::phoN2 cl 1 periplasmic extract in an in vitro ATP-degradation assay. In both assays, the EcN wild type strain (EcN) was used as negative control. The EcN wild type and EcN::phoN2 bacterial strains were grown in LB medium.

### Example 12: Recombinant Bacteria Encoding Apyrase in their Genome for Heterologous Expression Improve Dysbiosis-Induced Hypoglycemia and WAT Weight Loss

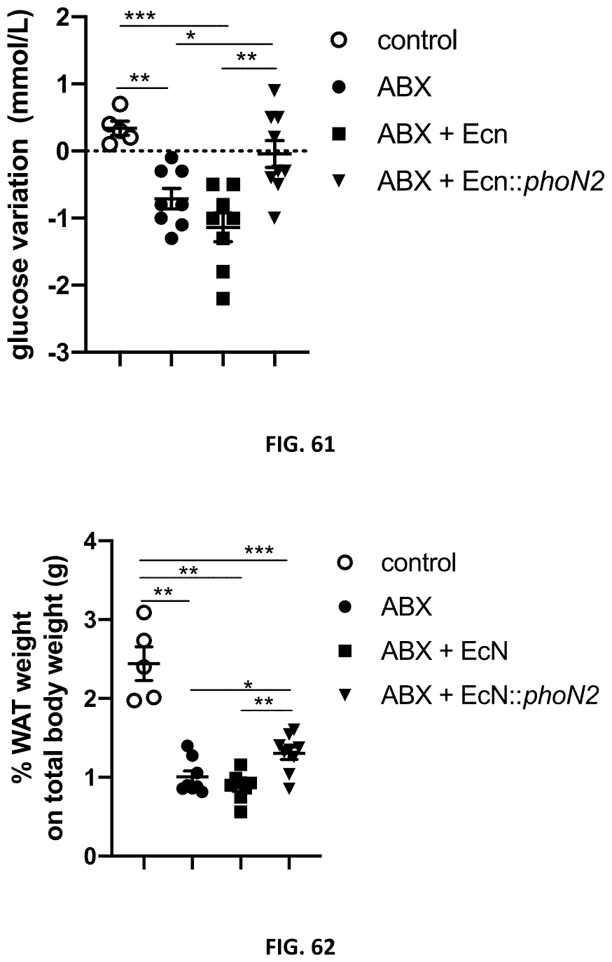

To investigate whether administration of E. coli Nissle 1917 (EcN) probiotic bacteria with phoN2 gene integrated in the genome (obtained as described above, Example 11) were effective in ameliorating hypoglycemia due to antibiotics-mediated dysbiosis, antibiotics (Vancomycin 1.25 mg, ampicillin 2.5 mg and metronidazole 1.25 mg in 200 μl sterile water per mouse) were administered to C57BL/6 mice that were subsequently gavaged with EcN or EcN::phoN2 strain (experimental schedule as shown in FIG. 60). Blood glucose was analysed after 4 days of antibiotic treatment and 4 days of recovery (day −4 and 3, respectively, in FIG. 60). As shown in FIG. 61, antibiotic treatment resulted in a significant decrease in blood glucose (hypoglycemia). Notably, mice treated with EcN::phoN2 showed serum glucose levels similar to untreated mice and significantly higher as compared to ABX and ABX+EcN treated mice. These data indicate that EcN::phoN2 administration restores blood glucose levels compromised by antibiotic treatments.

In order to evaluate the effect of EcN::phoN2 on antibiotics-mediated loss of WAT, white adipose tissue was collected and quantified. As shown in FIG. 62, WAT weight was significantly decreased due to antibiotics-induced dysbiosis, while this effect was attenuated in animals treated with EcN::phoN2, which showed significantly increased WAT weight than animals treated with ABX or ABX+EcN. These data indicate that EcN::phoN2 administration mitigates the antibiotics-induced WAT loss.

### Example 13: Recombinant Bacteria Encoding Apyrase in their Genome for Heterologous Expression Attenuate Caecum Enlargement and Bacterial Translocation to the Mesenteric Lymph Node (MLN) Caused by Dysbiosis

In order to investigate a possible beneficial effect of EcN::phoN2 in the recovery from dysbiosis, a mouse model of antibiotics-induced dysbiosis was used. The treatment schedule is shown in FIG. 60. 8-week old C57BL/6 male mice were randomly assigned to 4 different experimental groups: not treated (control), treated with antibiotics (ABX: Vancomycin 1.25 mg, ampicillin 2.5 mg and metronidazole 1.25 mg in 200 μl sterile water per mouse), treated with ABX and 10¹⁰ CFU of EcN and treated with ABX and 10¹⁰ CFU of EcN::phoN2. At the end of the experiment, mice were sacrificed by CO₂ inhalation and caecum and mesenteric lymph node (MLN) were harvested and analysed.

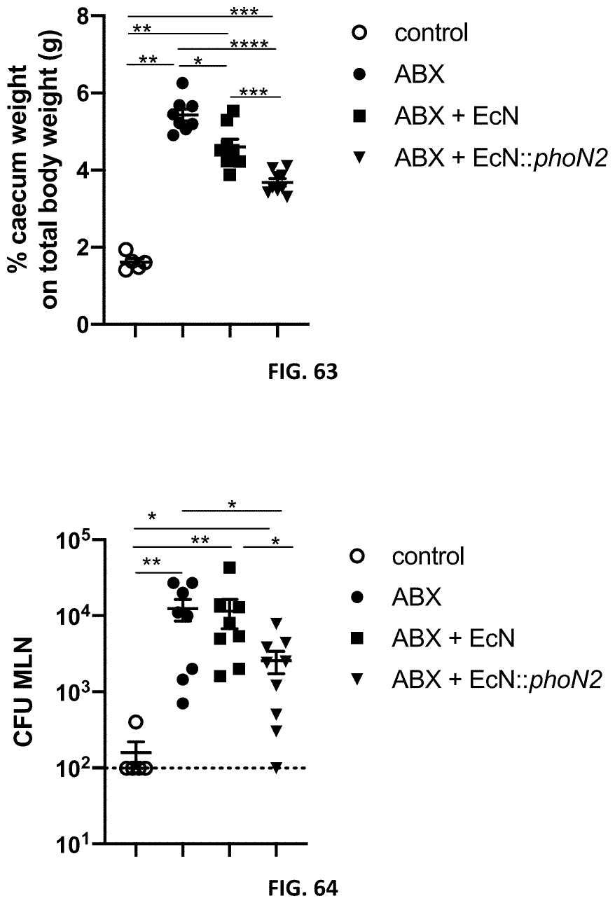

Caecum weight was analysed after 4 days of antibiotic treatment and 4 days of recovery (see FIG. 60). Results are shown in FIG. 63. Dysbiosis induced by antibiotics treatment resulted in a pronounced increased in caecum weight. However, mice treated with Ecn::phoN2 showed significantly lower caecum weight as compared to mice treated with ABX or ABX+EcN. These data indicate that EcN::phoN2 administration mitigates the antibiotic-mediated induction of caecum enlargement.

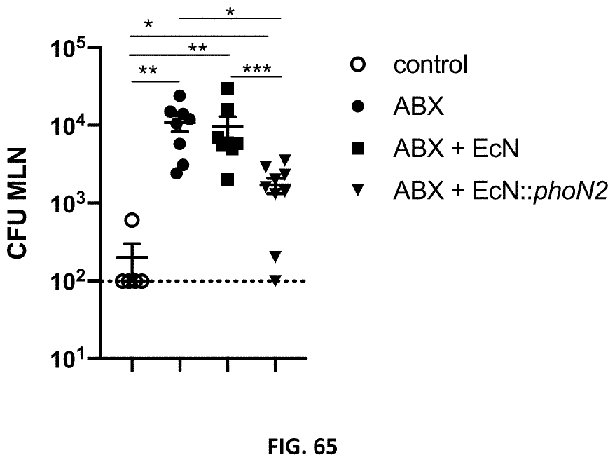

In order to investigate the effect of EcN::phoN2 in controlling bacterial translocation, MLN were harvested aseptically into RPMI and mechanically homogenized. Dilutions were plated onto Schaedler agar (BD Biosciences). Plates were grown under aerobic or anaerobic culture conditions at 37° C. for 24-72 h before enumeration of colonies. Results are shown in FIG. 64 and FIG. 65. Quantification of CFU from the MLN, both in aerobic and anaerobic conditions, revealed a significant increase in ABX and ABX+EcN treated mice compared to control animals. However, mice treated with EcN::phoN2 showed significantly reduced CFU both in aerobic and anaerobic conditions, compared to ABX and ABX+EcN treated mice. These data indicate that EcN::phoN2 administration attenuates bacterial translocation induced by antibiotics-mediated dysbiosis.

### Example 14: Design and Production of Apyrase Expressing Lactococcus lactis

To further expand our platform of apyrase expressing biotherapeutics, we selected Lactococcus lactis, as a Gram-positive strain. Due to its noninvasive and nonpathogenic characteristics, L. lactis has been demonstrated to be a promising candidate for the intestinal delivery of functional proteins (Varma, N. R., Toosa, H., Foo, H. L., Alitheen, N. B., Nor Shamsudin, M., Arbab, A. S., Yusoff, K., and Abdul Rahim, R. (2013). Display of the Viral Epitopes on Lactococcus lactis: A Model for Food Grade Vaccine against EV71. Biotechnol Res Int 2013, 431315). Genetically engineered L. lactis expressing interleukin-10 (IL-10) was used for the treatment of inflammatory bowel diseases (IBD) (Braat, H., Rottiers, P., Hommes, D. W., Huyghebaert, N., Remaut, E., Remon, J. P., van Deventer, S. J., Neirynck, S., Peppelenbosch, M. P., and Steidler, L. (2006). A phase I trial with transgenic bacteria expressing interleukin-10 in Crohn's disease. Clin Gastroenterol Hepatol 4, 754-759). Moreover, a recombinant L. lactis strain expressing the Pancreatitis-associated Protein (PAP) has been shown to efficiently preserve gut homeostasis in chemotherapy induced mucositis (Carvalho, R., Vaz, A., Pereira, F. L., Dorella, F., Aguiar, E., Chatel, J. M., Bermudez, L., Langella, P., Fernandes, G., Figueiredo, H., et al. (2018). Gut microbiome modulation during treatment of mucositis with the dairy bacterium Lactococcus lactis and recombinant strain secreting human antimicrobial PAP. Sci Rep 8, 15072).

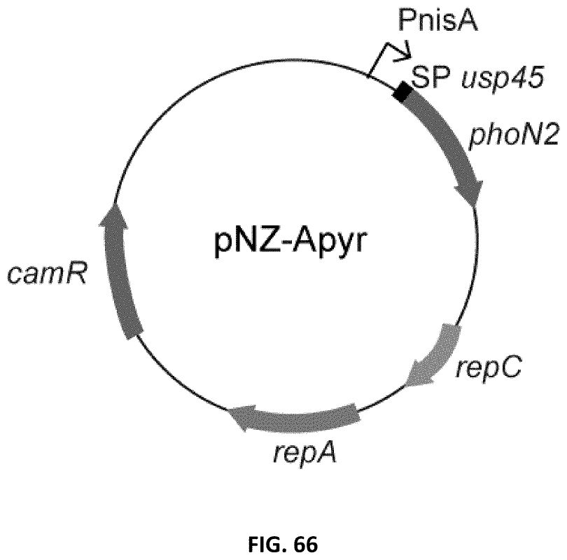

For the expression of Shigella flexneri apyrase in the Lactococcus lactis NZ900 strain, the apyrase encoding gene phoN2 was PCR amplified from the S. flexneri genome and cloned into the pNZ8123 plasmid, generating the pNZ-Apyr plasmid (FIG. 66). Apyrase expression in the pNZ-Apyr plasmid is controlled by the P_{nisA} promoter, which is inducible by the nisin anti-microbial peptide. The phoN2 gene was in-frame cloned with the signal sequence of the L. lactis major secreted protein Usp45 to allow apyrase secretion. L. lactis^{pNZ} and L. lactis^{pNZ-Apyr} strains were grown in M17 medium supplemented with glucose (0.5% w/v) and nisin (4 ng/ml).

### Example 15: Administration of L. lactis^{pNZ-Apyr} Counteracts Intestinal Barrier Disruption and Bacterial Translocation to MLN Caused by Diet Induced Dysbiosis in Adult Mice

The intestinal barrier defines the morpho-functional unit responsible for the defence of the intestinal mucosa and consists of the intestinal microbiota, intestinal epithelial cells (IECs) and mucosal immunity tightly linked through a complex network of cytokines, antimicrobial peptides (AMPs), metabolic products, and numerous regulatory molecules (Meng, M., Klingensmith, N. J., and Coopersmith, C. M. (2017). New insights into the gut as the driver of critical illness and organ failure. Curr Opin Crit Care 23, 143-148). The intestinal mucosa is the largest body surface at risk of infectious threats, the anatomic and functional homeostasis of the intestinal barrier is a key step in the anti-infectious defence of the human organism. The intestinal microbiota represents the first line of defence of the intestinal barrier. The microbiota entails millions of microorganisms colonizing the gastrointestinal tract most of which are bacteria. This large number of microorganisms withstands the unfavourable intestinal habitat thanks to the symbiotic relationships with the human organism. These symbiotic host-commensal relationships develop after birth and enable the metabolic, immune and anti-infectious processes through which the microbiota contributes to gut homeostasis (O'Hara, A. M., and Shanahan, F. (2006). The gut flora as a forgotten organ. EMBO Rep 7, 688-693). The structural and functional stability of commensal populations is regulated through numerous signaling molecules (quorum sensing) and cellular regulators (miRNAs) as well as through other physiologic and pathologic factors. Qualitative or quantitative alterations of this microbial community broadly defined as dysbiosis impair the relationships between the host and commensal species, modify the balance between commensals and pathogens, decrease the intestinal barrier protection and favour infectious pathogens (McDonald, D., Ackermann, G., Khailova, L., Baird, C., Heyland, D., Kozar, R., Lemieux, M., Derenski, K., King, J., Vis-Kampen, C., et al. (2016). Extreme Dysbiosis of the Microbiome in Critical Illness. mSphere 1: e00199-16. doi: 10.1128/mSphere.00199-16). Diet is a major element affecting the intestinal microbiota. Natural variations in food intake cause transient changes in microbial composition, although predominant components such as meat, fish, and fibers have durable effects on the microbiota and leave typical signatures characterized by shifts in specific bacterial groups (Scott, K. P., Gratz, S. W., Sheridan, P. O., Flint, H. J., and Duncan, S. H. (2013). The influence of diet on the gut microbiota. Pharmacol Res 69, 52-60). Changing food composition as well as food shortage or oversupply affect the gut microbiota. The absence of nutrients in the gut occurring in parenteral feeding increases the levels of Proteobacteria, which promote inflammation at the mucosal wall and eventually cause a breakdown of the epithelial barrier (Demehri, F. R., Barrett, M., and Teitelbaum, D. H. (2015). Changes to the Intestinal Microbiome With Parenteral Nutrition: Review of a Murine Model and Potential Clinical Implications. Nutr Clin Pract 30, 798-806). The influence of diet on the composition of the microbiota has been shown during the initial colonization phase: breast fed infants have higher levels of Bifidobacteria spp. while formula fed infants have higher levels of Bacteroides spp., as well as increased Clostridium coccoides and Lactobacillus spp. (Fallani, M., Young, D., Scott, J., Norin, E., Amarri, S., Adam, R., Aguilera, M., Khanna, S., Gil, A., Edwards, C. A., et al. (2010). Intestinal microbiota of 6-week-old infants across Europe: geographic influence beyond delivery mode, breast-feeding, and antibiotics. J Pediatr Gastroenterol Nutr 51, 77-84). Beyond the postnatal period, the microbiota was suspected to be relatively stable throughout life. However, several recent studies have shown that dietary factors alter the microbial community resulting in biological changes to the host. In fact, the composition of the gut microbiota strongly correlates with diet as demonstrated by a study assessing the relative contributions of host genetics and diet in shaping the gut microbiota and modulating metabolic syndrome phenotypes in mice (Zhang, C., Zhang, M., Wang, S., Han, R., Cao, Y., Hua, W., Mao, Y., Zhang, X., Pang, X., Wei, C., et al. (2010). Interactions between gut microbiota, host genetics and diet relevant to development of metabolic syndromes in mice. ISME J 4, 232-241).

Food is not only a source of nutrients but may also modulate some physiological functions of the body. This is especially true for the intestinal tract because of the continuous interaction of the intestine with dietary antigens (Ulluwishewa, D., Anderson, R. C., McNabb, W. C., Moughan, P. J., Wells, J. M., and Roy, N.C. (2011). Regulation of tight junction permeability by intestinal bacteria and dietary components. J Nutr 141, 769-776). Recent studies demonstrated the effects of the interaction between food and IECs. In fact, dietary antigens are able to modulate transporter activity, tight junction permeability, metabolic enzyme expression, immune functions, and microbiota (Shimizu, M. (2010). Interaction between food substances and the intestinal epithelium. Biosci Biotechnol Biochem 74, 232-241). Food entering the gastrointestinal tract provides nutrition to the organism. In addition, there are many metabolites produced by enzymatic conversion of nutrients, either by the host enzymes or by the gut microbiota, or by stimulating release of non-enzymatic molecules that influence diverse functions including alterations in the intestinal barrier.

The metabolites produced in the lumen may enter the bloodstream and reach sufficient concentrations to affect the functions of body organs (Dodd, D., Spitzer, M. H., Van Treuren, W., Merrill, B. D., Hryckowian, A. J., Higginbottom, S. K., Le, A., Cowan, T. M., Nolan, G. P., Fischbach, M. A., et al. (2017). A gut bacterial pathway metabolizes aromatic amino acids into nine circulating metabolites. Nature 551, 648-652). These interactions between diet, digestion, microbiota, and the intestinal barrier may impact gut homeostasis (Farre, R., Fiorani, M., Abdu Rahiman, S., and Matteoli, G. (2020). Intestinal Permeability, Inflammation and the Role of Nutrients. Nutrients 12:1185. doi: 10.3390/nu12041185).

Diet induced dysbiosis trigger also mechanisms that unbalance the intestinal homeostasis and cause inflammation. The translocation of bacteria across the gut epithelium increases in dysbiosis (Sato, J., Kanazawa, A., Ikeda, F., Yoshihara, T., Goto, H., Abe, H., Komiya, K., Kawaguchi, M., Shimizu, T., Ogihara, T., et al. (2014). Gut dysbiosis and detection of “live gut bacteria” in blood of Japanese patients with type 2 diabetes. Diabetes Care 37, 2343-2350). Small numbers of translocated commensal bacteria, as they occur in a healthy human gut, are removed by the action of Th1 and Th17 cells that are particularly induced by polysaccharides of Bacteroides spp. (Mazmanian, S. K., and Kasper, D. L. (2006). The love-hate relationship between bacterial polysaccharides and the host immune system. Nat Rev Immunol 6, 849-858) and mucosa-adherent segmented filamentous bacteria (SFB) (Ivanov, I I, Atarashi, K., Manel, N., Brodie, E. L., Shima, T., Karaoz, U., Wei, D., Goldfarb, K. C., Santee, C. A., Lynch, S. V., et al. (2009). Induction of intestinal Th17 cells by segmented filamentous bacteria. Cell 139, 485-498). Conversely, high numbers of invading bacteria activate TLRs and elicit an overexpression of pro-inflammatory cytokines, which damage the gut epithelium and lead to chronic intestinal inflammation (Karczewski, J., Poniedzialek, B., Adamski, Z., and Rzymski, P. (2014). The effects of the microbiota on the host immune system. Autoimmunity 47, 494-504). Disruption of the intestinal barrier, increased intestinal permeability and translocation of bacterial antigens towards metabolically active tissue would result in a chronic inflammatory state and impaired metabolic functions such as insulin resistance, hepatic fat deposition, excessive adipose tissue development (Tehrani, A. B., Nezami, B. G., Gewirtz, A., and Srinivasan, S. (2012). Obesity and its associated disease: a role for microbiota? Neurogastroenterol Motil 24, 305-311; Camilleri, M. (2019). Leaky gut: mechanisms, measurement and clinical implications in humans. Gut 68, 1516-1526), rheumatoid arthritis (Guerreiro, C. S., Calado, A., Sousa, J., and Fonseca, J. E. (2018). Diet, Microbiota, and Gut Permeability—The Unknown Triad in Rheumatoid Arthritis. Front Med (Lausanne) 5, 349) and ulcerative colitis (Den Hond, E., Hiele, M., Evenepoel, P., Peeters, M., Ghoos, Y., and Rutgeerts, P. (1998). In vivo butyrate metabolism and colonic permeability in extensive ulcerative colitis. Gastroenterology 115, 584-590).

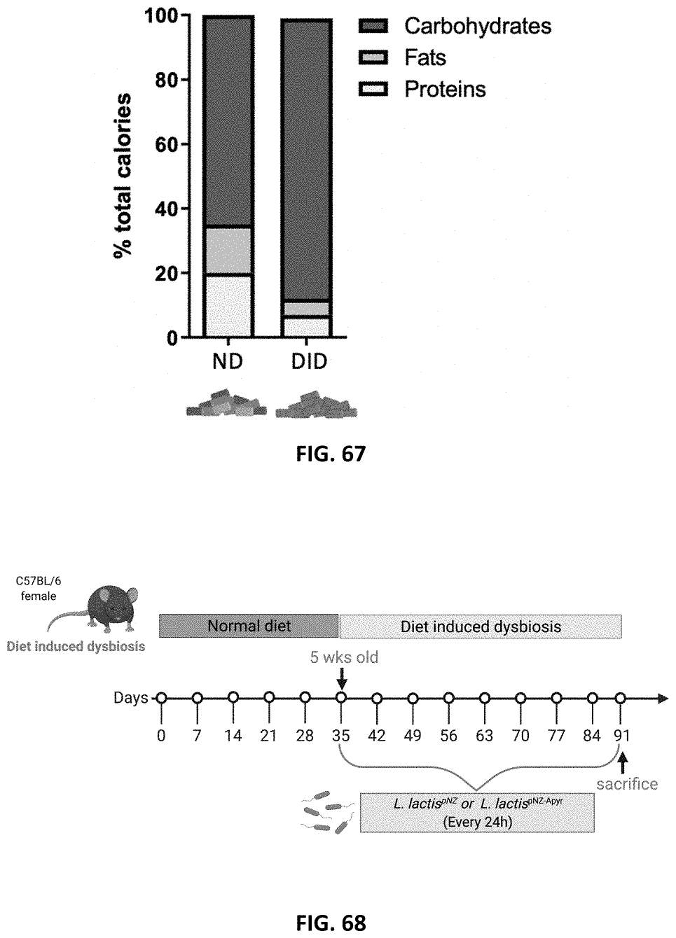

A mouse model of diet induced dysbiosis (DID) was created to investigate possible beneficial effects of apyrase on this condition. Dysbiosis was induced by feeding mice with a diet characterized by 7% protein, 5% fat and 88% carbohydrates. Normal diet (ND) characterized by 20% protein, 15% fat and 65% carbohydrate was used as control. A schematic of the components of each diet is shown in FIG. 67. Experimental layout showed in FIG. 68 shows the DID model in 5 weeks old mice. At 5 weeks of age, female C67BL/6 mice were randomized into receiving either ND or DID diet. During this period, DID diet fed mice were orally gavaged every day with 10¹⁰ of L. lactis^{pNZ} or L. lactis^{pNZ-Apyr}. After 8 weeks, mice were sacrificed and analyzed in order to evaluate the effects of the different diets.

DID is characterized by a modification of gut microbiota that can impair the gut barrier functions of the intestinal mucosa, leading to enhanced mucosa permeability and subsequent translocation of commensal bacteria and or bacterial products into blood circulation (Fukui, H. (2019). Role of Gut Dysbiosis in Liver Diseases: What Have We Learned So Far? Diseases 7:58 doi: 10.3390/diseases7040058).

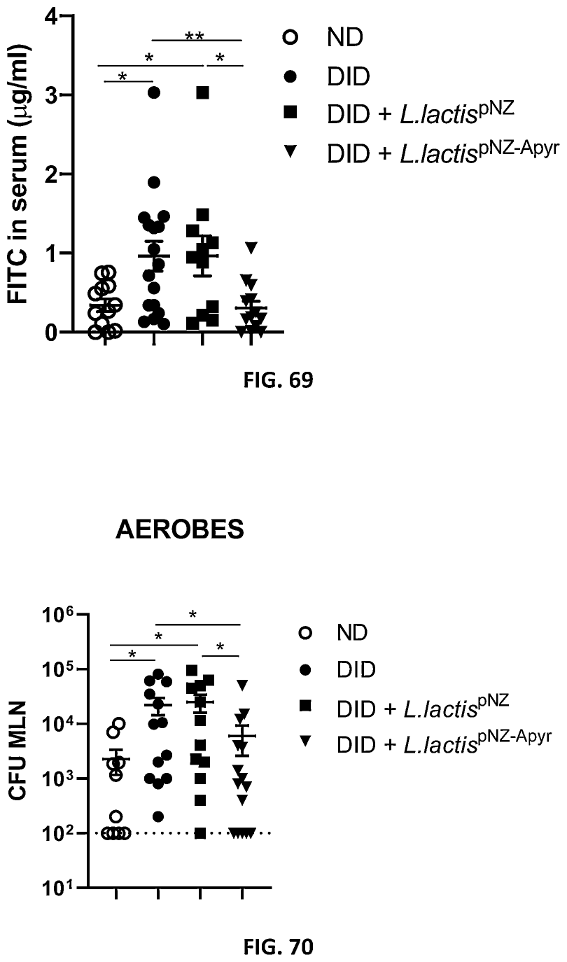

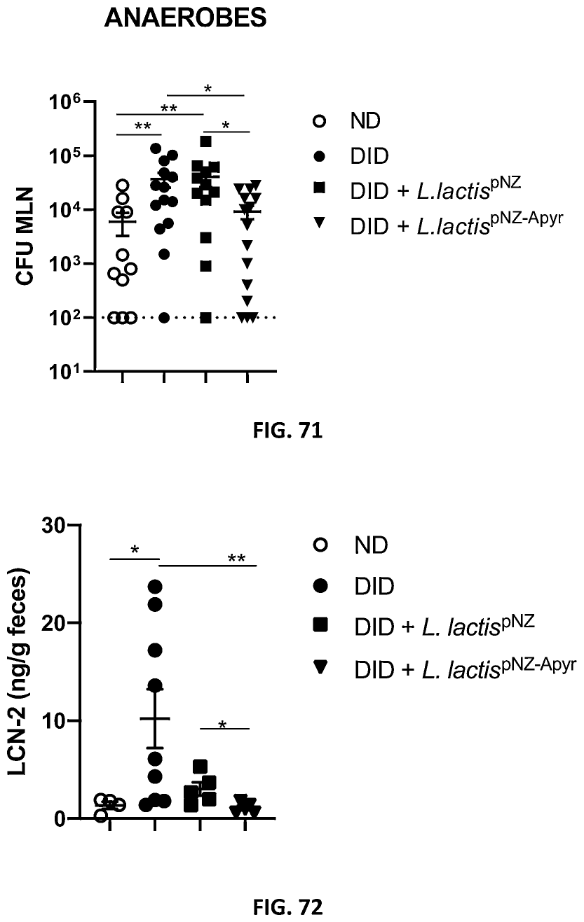

In order to investigate if L. lactis^{pNZ-Apyr} could preserve intestinal integrity, dysbiosis was induced and bacteria expressing apyrase were administered as described above (experimental schedule as shown in FIG. 68). After 8 weeks of ND or DID diet, mice were orally gavaged with fluorescein isothiocyanate (FITC)-labeled dextran and subsequently FITC levels were measured in the serum. Results are shown in FIG. 69. DID mice either treated or not with L. lactis^{pNZ} were characterized by significantly higher concentrations of FITC in serum compared to ND mice. In contrast, mice fed with DID diet and treated with L. lactis^{pApyr} showed levels of FITC in serum that were comparable to ND animals and significantly lower with respect to mice fed with stand-alone DID diet or in association with L. lactis^{pNZ}. These data indicate that L. lactis^{pNZ-Apyr} administration attenuates gut barrier disruption caused by DID. Moreover, in order to investigate the effect of apyrase in the mitigation of bacterial translocation to the MLN, mice were sacrificed at the end of the experiment (see FIG. 68), MLN were harvested aseptically into RPMI and mechanically homogenized. Dilutions were plated onto Schaedler agar (BD Biosciences). Plates were grown under aerobic or anaerobic culture conditions at 37° C. for 24-72 h before enumeration of colonies. Results are shown in FIG. 70 and FIG. 71. Quantification of CFU from the MLN, both in aerobic and anaerobic conditions, revealed a significant increase of MLN CFU in DID diet or DID diet+L. lactis^{pNZ} treated mice compared to ND animals. In contrast, DID mice treated with L. lactis^{pNZ-Apyr} showed a number of CFU in the MLN that was not significantly different from ND animals and significantly lower with respect to DID diet and DID diet+L. lactis^{pNZ} mice. These data indicate that L. lactis^{pNZ-Apyr} administration attenuates bacterial translocation induced by DID.

Concomitantly, DID and DID+L. lactis^{pNZ} mice developed mild signs of intestinal inflammation quantified as increased levels of lipocalin-2 (LCN-2) in the stools compared to ND mice. Strikingly, mice fed with DID diet and treated with L. lactis^{pNZ-Apyr} showed levels of fecal LCN-2 that were not significantly different from ND animals and significantly lower with respect to mice fed with stand-alone DID diet or in association with L. lactis^{pNZ}. These data indicate that L. lactis^{pNZ-Apyr} administration attenuates intestinal inflammation caused by DID (results are shown in FIG. 72).

### Example 16: Administration of L. lactis^{pNZ-Apyr} Counteracts Intestinal Barrier Disruption and Bacterial Translocation to MLN Caused by Diet Induced Dysbiosis in Neonatal Mice

Transmission of metabolic diseases from mother to child is multifactorial and includes genetic, epigenetic and environmental influences. Evidence in rodents, humans and non-human primates support the scientific premise that exposure to diet induced dysbiosis during pregnancy creates a long-lasting metabolic signature on the infant immune system and juvenile microbiota, which predisposes the offspring to obesity and metabolic diseases. In neonates, gastrointestinal microbes introduced through the mother are noted for their ability to serve as direct inducers/regulators of the infant immune system. Neonates have a limited capacity to initiate an immune response. Thus, disruption of microbial colonization during the early neonatal period results in disrupted postnatal immune responses. Although the mechanisms are poorly understood, increasing evidence suggests that diet induced dysbiosis during pregnancy influences the development and modulation of the infant microbiota composition, liver and other organs through direct communication via the portal system, metabolite production, alterations in gut barrier integrity and the hematopoietic immune cell axis (Collado, M. C., Isolauri, E., Laitinen, K., and Salminen, S. (2010). Effect of mother's weight on infant's microbiota acquisition, composition, and activity during early infancy: a prospective follow-up study initiated in early pregnancy. Am J Clin Nutr 92, 1023-1030; Collado, M. C., Laitinen, K., Salminen, S., and Isolauri, E. (2012). Maternal weight and excessive weight gain during pregnancy modify the immunomodulatory potential of breast milk. Pediatr Res 72, 77-85).

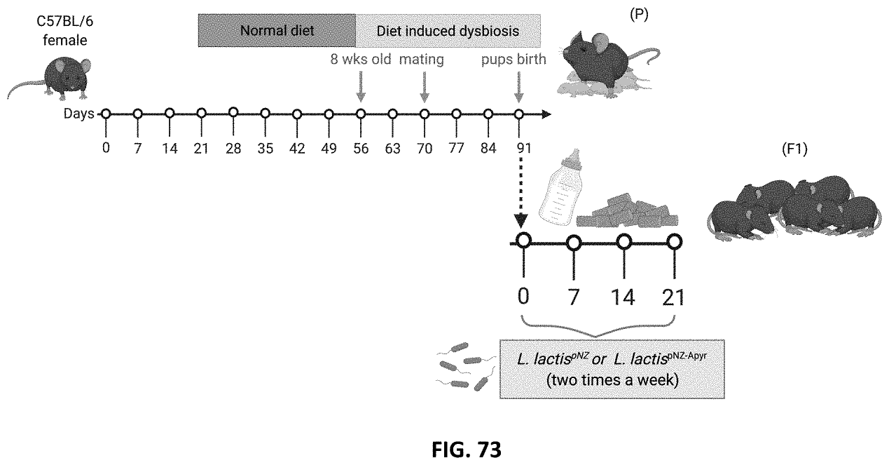

A neonatal model of diet-induced dysbiosis (DID) was created to investigate possible beneficial effect of apyrase on DID in neonates. Dysbiosis was induced starting from the mothers that were fed with a diet characterized by 7% protein, 5% fat and 88% carbohydrates. A normal diet (ND) characterized by 20% protein, 15% fat and 65% carbohydrate was used as control. A schematic of the components of each diet is shown in FIG. 67. Experimental layout in FIG. 73 shows the neonatal model of DID. At eight weeks of age, female C57BL/6 mice were randomized into receiving either a ND or a DID diet. After 15 days, ND and DID female C57BL/6 mice were mated with a ND male mice. Starting immediately after birth, DID pups were orally gavaged with 10⁸ of L. lactis^{pNZ} or L. lactis^{pNZ-Apyr} two times a week until 21 days after birth. Pups were daily monitored for body weight, tail length and behavior until 21 days after birth.

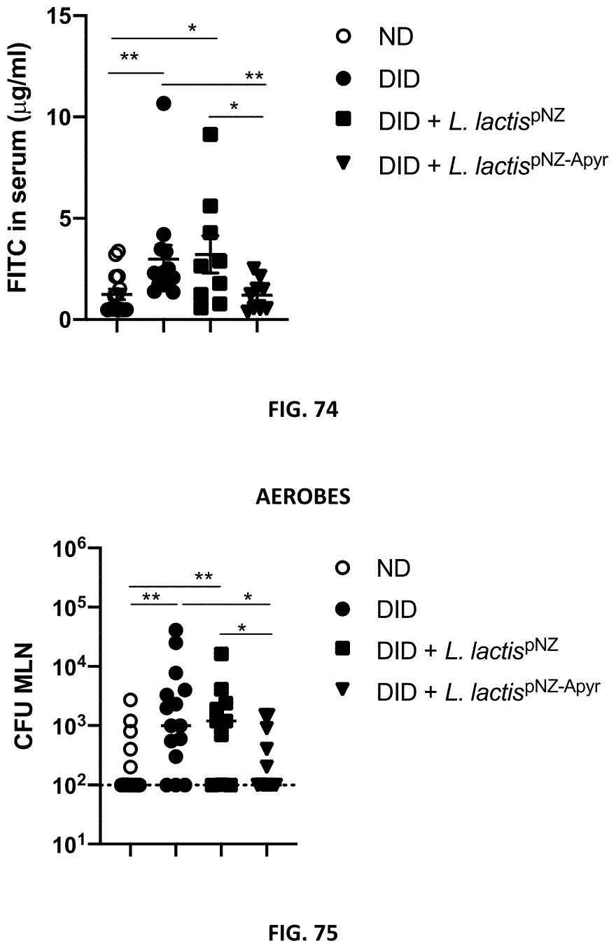

In order to investigate if L. lactis^{pNZ-Apyr} could preserve intestinal integrity, 21 days old DID or ND mice were orally gavaged with fluorescein isothiocyanate (FITC)-labeled dextran and subsequently FITC levels were measured in the serum. Results are shown in FIG. 74. DID and DID+L. lactis^{pNZ} mice were characterized by higher concentration of FITC in serum compared to ND mice. In contrast, mice fed with DID diet and treated with L. lactis^{pNZ-Apyr} showed levels of FITC in serum that were not significantly different from ND animals, while they were significantly lower with respect to mice fed with stand-alone DID diet or in association with L. lactis^{pNZ}. These data indicate that L. lactis^{pNZ-Apyr} administration attenuates gut barrier disruption caused by diet induced dysbiosis.

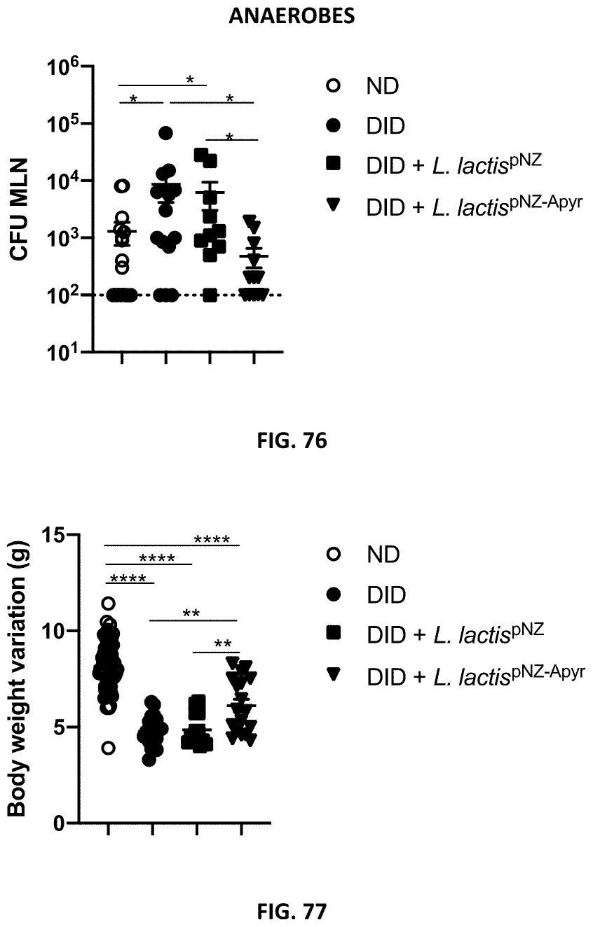

Moreover, in order to investigate the effect of apyrase in the mitigation of bacterial translocation to the MLN, mice were sacrificed at the end of the experiment (see FIG. 73), MLN were harvested aseptically into RPMI and mechanically homogenized. Dilutions were plated onto Schaedler agar (BD Biosciences). Plates were grown under aerobic or anaerobic culture conditions at 37° C. for 24-72 h before enumeration of colonies. Results are shown in FIG. 75 and FIG. 76. Quantification of CFU in the MLN, both in aerobic and anaerobic conditions, revealed a significant increase of CFU in DID or DID+L. lactis^{pNZ} mice compared to the ND animals. In contrast, DID mice treated with L. lactis^{pNZ-Apyr} showed a number of CFU in the MLN that was at all similar to control animals and significantly lower with respect to DID and DID+L. lactis^{pNZ} mice. These data indicate that L. lactis^{pNZ-Apyr} administration counteracts bacterial translocation induced by diet induced dysbiosis.

### Example 17: Administration of L. lactis^{pNZ-Apyr} Improves Growth Parameters Affected by Diet Induced Dysbiosis in Neonatal Mice

Maternal protein deficiency causes severe dysbiosis that results in fetal growth retardation and predisposition to diseases in adult life (Rees, W. D., Hay, S. M., Buchan, V., Antipatis, C., and Palmer, R. M. (1999). The effects of maternal protein restriction on the growth of the rat fetus and its amino acid supply. Br J Nutr 81, 243-250.).

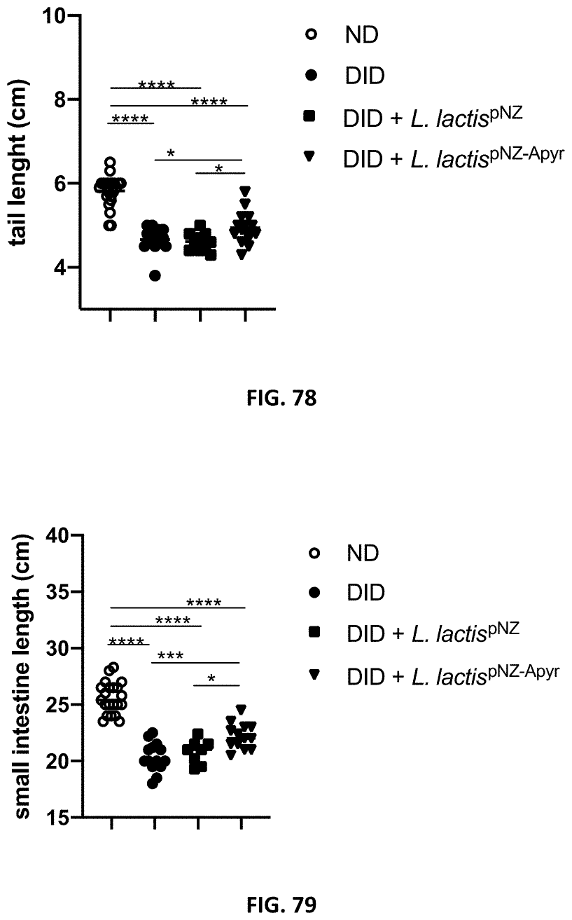

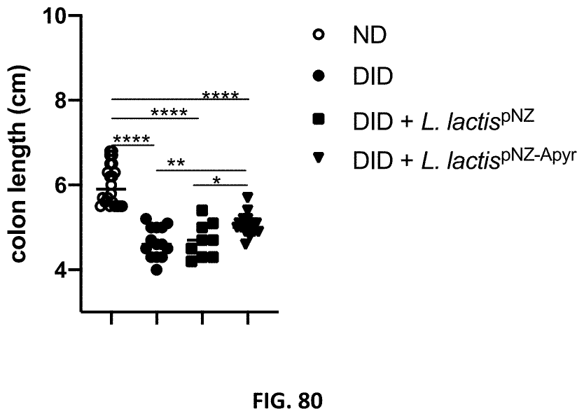

In order to understand if apyrase could have an impact on growth parameters in the offspring, neonates born from ND and DID dams were orally gavaged with 10⁸ of L. lactis^{pNZ} or L. lactis^{pNZ-Apyr} two times a week until 21 days after birth (see FIG. 73). At day 21 after birth, body weight, tail length, small intestine and colon length were evaluated. Diet induced dysbiosis in the mothers significantly affected body weight variation (FIG. 77), tail length (FIG. 78), small intestine (FIG. 79) and colon length (FIG. 80) in both DID and DID+L. lactis^{pNZ} neonates. Notably, DID neonates treated with L. lactis^{pNZ-Apyr} showed an amelioration of all the different growth parameters compared to the others DID groups. Therefore, L. lactis^{pNZ-Apyr} administration improves the growth retardation caused by DID.

TABLE OF
SEQUENCES AND SEQ ID NUMBERS (SEQUENCE LISTING):

| SEQ ID NO | Sequence | Remarks |
|---|---|---|
| SEQ ID NO: 1 | MKTKNFLLFCIATNMIFIPSANALKAEGFLTQQTSPDSLSI | Apyrase |
| | LPPPPAEDSVVFLADKAHYEFGRSLRDANRVRLASEDAY | |
| | YENFGLAFSDAYGMDISRENTPILYQLLTQVLQDSHDYA | |
| | VRNAKEYYKRVRPFVIYKDATCTPDKDEKMAITGSYPSG | |
| | HASFGWAVALILAEINPQRKAEILRRGYEFGESRVICGAH | |
| | WQSDVEAGRLMGASVVAVLHNTPEFTKSLSEAKKEFEEL | |
| | NTPTNELTP | |
| SEQ ID NO: 2 | MKTKNFLLFCIATNMIFIPSANALKAEGFLTQQTSPDSLSI | Loss-of- |
| | LPPPPAEDSVVFLADKAHYEFGRSLRDANRVRLASEDAY | function |
| | YENFGLAFSDAYGMDISRENTPILYQLLTQVLQDSHDYA | isoform |
| | VRNAKEYYKRVRPFVIYKDATCTPDKDEKMAITGSYPSG | of apyrase |
| | HASFGWAVALILAEINPQRKAEILRRGYEFGESPVICGAH | |
| | WQSDVEAGRLMGASVVAVLHNTPEFTKSLSEAKKEFEEL | |
| | NTPTNELTP | |
| SEQ ID NO: 3 | ATGAAAACCAAAAACTTTCTTCTTTTTTGTATTGCTACA | phoN2 gene |
| | AATATGATTTTTATCCCCTCAGCAAATGCTCTGAAGGC | encoding |
| | AGAAGGTTTTCTCACTCAACAAACTTCACCAGACAGTT | apyrase |
| | TGTCAATACTTCCGCCGCCTCCGGCAGAGGATTCAGT | |
| | AGTATTTCTGGCTGACAAAGCTCATTATGAATTCGGCC | |
| | GCTCGCTCCGGGATGCTAATCGTGTACGTCTCGCTAG | |
| | CGAAGATGCATACTACGAGAATTTTGGTCTTGCATTTT | |
| | CAGATGCTTATGGCATGGATATTTCAAGGGAAAATAC | |
| | CCCAATCTTATATCAGTTGTTAACACAAGTACTACAGG | |
| | ATAGCCATGATTACGCCGTGCGTAACGCCAAAGAATA | |
| | TTATAAAAGAGTTCGTCCATTCGTTATTTATAAAGACG | |
| | CAACCTGTACACCTGATAAAGATGAGAAAATGGCTAT | |
| | CACTGGCTCTTATCCCTCTGGTCATGCATCCTTTGGTT | |
| | GGGCAGTAGCACTGATACTTGCGGAGATTAATCCTCA | |
| | ACGTAAAGCGGAAATACTTCGACGTGGATATGAGTTT | |
| | GGAGAAAGTCGGGTCATCTGCGGTGCGCATTGGCAA | |
| | AGCGATGTAGAGGCTGGGCGTTTAATGGGAGCATCG | |
| | GTTGTTGCAGTACTTCATAATACACCTGAATTTACCAA | |
| | AAGCCTTAGCGAAGCCAAAAAAGAGTTTGAAGAATTA | |
| | AATACTCCTACCAATGAACTGACCCCATAA | |
| SEQ ID NO: 4 | CCTACGGGNGGCWGCAG | forward primer |
| SEQ ID NO: 5 | GACTACHVGGGTATCTAATCC | reverse primer |
| SEQ ID NO: 6 | CGAGCAGGCACACTGGAAGTATTGCTGCATCAGGCG | EcN malP gene |
| | CAGCTTTTTACCGGCAGTATGGTTGTCGTTTGGATAGA | portion |
| | GAACTTTGGTCAGTTTTTCCGCGTTGATGCCCTGCTGT | |
| | TCGGCACGCAGGAAATCACCGTCGTTAAATTTAGTCA | |
| | GATCAAACGGATGCGCATGCGTCGCCTGCCACAGACG | |
| | CAGTGGCTGCGCCACGCCATTACGATAGCCGACAACG | |
| | GGGAGATCCCACGCTTGACCGGTAATGGTAAACTCCG | |
| | GCTCCCAGCGTCCATCTTTCGTCACTTTACCGCCAATC | |
| | CCTACCTGCACATCCAGTGCTTCGTTGTGGCGGAACC | |
| | ACGGGTAGTTACCGCGATGCCAGTCATCCGGCGCTTC | |
| | AACCTGTTTGCCATCGACAAATGACTGGCGGAACAAG | |
| | CCATATTGATAATTAAGGCCGTAGCCAGTAGCTGACT | |
| | GCCCGACAGTTGCCATTGAGTCGAGGAAGCACGCCG | |
| | CCAGACGTCCCAGACCACCGTTCCCCAGCGCCGGGTC | |
| | GATCTCTTCTTCCAACAGGTCAGTCAGGTTGATGTCAT | |
| | AAGCCTTCAACGAATCCTGTACATCCTGATACCAGCCG | |
| | AGATTCAACAGGTTGTTGCCCGTCAGGCGACCAATCA | |
| | AAAACTCCATTGAGATGTAGTTAACATGTCGCTGATTC | |
| | GCCACTGGCTTGGCGAATGGCTGAGCACGCAGCATTT | |
| | CGGCCAGTGCTTCGCTCACTGCCAGCCACCACTGGCG | |
| | AGGAGTCATTTCAGCCGCAGAATTTAAGCCATAACGC | |
| | TGCCACTGACGTGAAAGCGCTTCCTGAAATTGCTTATC | |
| | GTTAAAAATAGGTTGTGACAT | |
| SEQ ID NO: 7 | ATGCTGATTCCGTCAAAATTAAGTCGTCCGGTTCGACT | EcN malT gene |
| | CGACCATACCGTGGTTCGTGAGCGCCTGCTGGCTAAA | portion |
| | CTTTCCGGCGCGAACAACTTCCGGCTGGCGCTGATCA | |
| | CAAGTCCTGCGGGCTACGGAAAGACCACGCTCATTTC | |
| | CCAGTGGGCGGCAGGCAAAAACGATATCGGCTGGTA | |
| | CTCGCTGGATGAAGGTGATAACCAGCAAGAGCGTTTC | |
| | GCCAGCTATCTCATTGCCGCCGTGCAACAGGCAACCA | |
| | ACGGTCACTGCGCGATATGTGAGACGATGGCGCAAA | |
| | AACGGCAATATGCCAGCCTGACGTCACTCTTCGCCCA | |
| | GCTTTTCATTGAGCTGGCGGAATGGCATAGCCCACTTT | |
| | ATCTGGTCATCGATGACTATCATCTGATCACTAATCCT | |
| | GTGATCCACGAGTCAATGCGCTTCTTTATTCGCCATCA | |
| | ACCAGAAAATCTCACCCTTGTGGTGTTGTCACGCAACC | |
| | TTCCGCAACTGGGCATTGCCAATCTGCGTGTTCGTCCA | |
| | GCTAGCGAATTCGCTGGAAATTGGCAGTCAGCAACTG | |
| | GCATTTACCCATCAGGAAGCGAAGCAGTTTTTTGATT | |
| | GCCGTCTGTCATCGCCGATTGAAGCTGCAGAAAGCAG | |
| | TCGGATTTGTGATGATGTTTCCGGTTGGGCGACGGCA | |
| | CTGCAGCTAATCGCCCTCTCCGCCCGGCAGAATACTCA | |
| | CTCAGCCCATAAGTCGGCACGCCGCCTGGCGGGAATC | |
| | AATGCCAGCCATCTTTCGGATTATCTGGTCGATGAGG | |
| | TTTTGGATAACGTCGATCTCGCAACGCGCCA | |
| SEQ ID NO: 8 | CAGCTAACACCACGTCGTCCCTATCTGCTGCCCTAGGT | DNA fragment |
| | CTATGAGTGGTTGCTGGATAACTTTACGGGCATGCAT | including |
| | AAGGCTCGTATAATATATTCAGGGAGACCACAACGGT | the P_{proD} |
| | TTCCCTCTACAAATAATTTTGTTTAACTTTTACTAGAGT | promoter, |
| | CACACAGGAAAGTACTAGATGAAAACCAAAAACTTTC | the BBa_BB0032 |
| | TTCTTTTTTGTATTGCTACAAATATGATTTTTATCCCCTC | RBS, the |
| | AGCAAATGCTCTGAAGGCAGAAGGTTTTCTCACTCAA | S. flexneri |
| | CAAACTTCACCAGACAGTTTGTCAATACTTCCGCCGCC | phoN2 gene |
| | TCCGGCAGAGGATTCAGTAGTATTTCTGGCTGACAAA | and the phoN2 |
| | GCTCATTATGAATTCGGCCGCTCGCTCCGGGATGCTA | transcriptional |
| | ATCGTGTACGTCTCGCTAGCGAAGATGCATACTACGA | terminator |
| | GAATTTTGGTCTTGCATTTTCAGATGCTTATGGCATGG | |
| | ATATTTCAAGGGAAAATACCCCAATCTTATATCAGTTG | |
| | TTAACACAAGTACTACAGGATAGCCATGATTACGCCG | |
| | TGCGTAACGCCAAAGAATATTATAAAAGAGTTCGTCC | |
| | ATTCGTTATTTATAAAGACGCAACCTGTACACCTGATA | |
| | AAGATGAGAAAATGGCTATCACTGGCTCTTATCCCTCT | |
| | GGTCATGCATCCTTTGGTTGGGCAGTAGCACTGATAC | |
| | TTGCGGAGATTAATCCTCAACGTAAAGCGGAAATACT | |
| | TCGACGTGGATATGAGTTTGGAGAAAGTCGGGTCATC | |
| | TGCGGTGCGCATTGGCAAAGCGATGTAGAGGCTGGG | |
| | CGTTTAATGGGAGCATCGGTTGTTGCAGTACTTCATA | |
| | ATACACCTGAATTTACCAAAAGCCTTAGCGAAGCCAA | |
| | AAAAGAGTTTGAAGAATTAAATACTCCTACCAATGAA | |
| | CTGACCCCATAAAGCTGGACAGCCTGT [symbol] | |
| | [symbol] [symbol] [symbol] | |
| | [symbol] [symbol] | |
| | [symbol] [symbol] [symbol] | |
| | [symbol] [symbol] [symbol] | |
| | [symbol] [symbol] [symbol] | |
| | [symbol] [symbol] | |
| | [symbol] [symbol] [symbol] | |
| | [symbol] [symbol] [symbol] | |
| | [symbol] [symbol] [symbol] | |
| | [symbol] [symbol] | |
| SEQ ID NO: 9 | GAAGTTCCTATTCTCTAGAAAGTATAGGAACTTCGGC | DNA fragment |
| | GCGCCTACCTGTGACGGAAGATCACTTCGCAGAATAA | including the |
| | ATAAATCCTGGTGTCCCTGTTGATACCGGGAAGCCCT | E. coli cat |
| | GGGCCAACTTTTGGCGAAAATGAGACGTTGATCGGCA | gene flanked |
| | CGTAAGAGGTTCCAACTTTCACCATAATGAAATAAGA | by the |
| | TCACTACCGGGCGTATTTTTTGAGTTGTCGAGATTTTC | FRT sequences |
| | AGGAGCTAAGGAAGCTAAAATGGAGAAAAAAATCAC | |
| | TGGATATACCACCGTTGATATATCCCAATGGCATCGTA | |
| | AAGAACATTTTGAGGCATTTCAGTCAGTTGCTCAATGT | |
| | ACCTATAACCAGACCGTTCAGCTGGATATTACGGCCTT | |
| | TTTAAAGACCGTAAAGAAAAATAAGCACAAGTTTTAT | |
| | CCGGCCTTTATTCACATTCTTGCCCGCCTGATGAATGC | |
| | TCATCCGGAATTACGTATGGCAATGAAAGACGGTGAG | |
| | CTGGTGATATGGGATAGTGTTCACCCTTGTTACACCGT | |
| | TTTCCATGAGCAAACTGAAACGTTTTCATCGCTCTGGA | |
| | GTGAATACCACGACGATTTCCGGCAGTTTCTACACATA | |
| | TATTCGCAAGATGTGGCGTGTTACGGTGAAAACCTGG | |
| | CCTATTTCCCTAAAGGGTTTATTGAGAATATGTTTTTC | |
| | GTCTCAGCCAATCCCTGGGTGAGTTTCACCAGTTTTGA | |
| | TTTAAACGTGGCCAATATGGACAACTTCTTCGCCCCCG | |
| | TTTTCACCATGGGCAAATATTATACGCAAGGCGACAA | |
| | GGTGCTGATGCCGCTGGCGATTCAGGTTCATCATGCC | |
| | GTTTGTGATGGCTTCCATGTCGGCAGAATGCTTAATG | |
| | AATTACAACAGTACTGCGATGAGTGGCAGGGCGGGG | |
| | CGTAAGGCGCGCCATTTAAATGAAGTTCCTATTCCGA | |
| | AGTTCCTATTCTCTAGAAAGTATAGGAACTTCGAAGCA | |
| | GCTCCAGCCTACACAATGAATTC | |

1. An ATP hydrolyzing enzyme, a nucleic acid comprising a polynucleotide encoding the ATP hydrolyzing enzyme, a host cell comprising the nucleic acid, a microorganism comprising the nucleic acid, a (recombinant) bacterium comprising the nucleic acid, or a viral particle comprising the nucleic acid
for use in the treatment of dysbiosis or a dysbiosis-related disease.

2. An ATP hydrolyzing enzyme, a nucleic acid comprising a polynucleotide encoding the ATP hydrolyzing enzyme, a host cell comprising the nucleic acid, a microorganism comprising the nucleic acid, a (recombinant) bacterium comprising the nucleic acid, or a viral particle comprising the nucleic acid
for use in restoring or improving the microbiome balance during or after dysbiosis.

3. An ATP hydrolyzing enzyme for use in the treatment of dysbiosis or a dysbiosis-related disease.

4. The ATP hydrolyzing enzyme for use according to any one of claims 1 to 3, wherein the ATP hydrolyzing enzyme is a soluble ATP hydrolyzing enzyme.

5. The ATP hydrolyzing enzyme for use according to any one of the previous claims, wherein the ATP hydrolyzing enzyme is apyrase.

6. The ATP hydrolyzing enzyme for use according to claim 5, wherein the apyrase is a bacterial apyrase or a plant apyrase.

7. The ATP hydrolyzing enzyme for use according to any one of the previous claims, wherein the ATP hydrolyzing enzyme comprises an amino acid sequence as set forth in SEQ ID NO: 1 or a sequence variant thereof having at least 70%, 80% or 90% sequence identity.

8. A nucleic acid comprising a polynucleotide encoding the ATP hydrolyzing enzyme as defined in any one of claims 4-7 for use in the treatment of dysbiosis or a dysbiosis-related disease.

9. The nucleic acid for use according to claim 1, 2 or 7, wherein the nucleic acid comprising the polynucleotide encoding the ATP hydrolyzing enzyme is a vector.

10. The nucleic acid for use according to any one of claims 1, 2, 7 or 8, wherein the nucleic acid further comprises heterologous elements for (heterologous) expression of the ATP hydrolyzing enzyme.

11. A host cell comprising the nucleic acid as defined in any one of claims 8-10 for use in the treatment of dysbiosis or a dysbiosis-related disease.

12. The host cell for use according to claim 1, 2 or 11, wherein the host cell is a prokaryotic or a eukaryotic cell.

13. The host cell for use according to claim 1, 2, 11 or 12, wherein the host cell is a recombinant host cell heterologously expressing the ATP hydrolyzing enzyme.

14. A microorganism comprising the nucleic acid as defined in any one of claims 8-10 for use in the treatment of dysbiosis or a dysbiosis-related disease.

15. The microorganism for use according to claim 1, 2 or 14, wherein the microorganism is selected from archaea, bacteria and eukaryotes.

16. The microorganism for use according to claim 1, 2, 14 or 15, wherein the microorganism is selected from the group consisting of Escherichia spp., Salmonella spp., Yersinia spp., Vibrio spp., Listeria spp., Lactococcus spp., Shigella spp., Cyanobacteria, and Saccharomyces spp.

17. The microorganism for use according to any one of claims 1, 2 and 14 to 16, wherein the microorganisms are provided as probiotics.

18. The microorganism for use according to any one of claims 1, 2 and 14 to 17, wherein the virulence of the microorganism is attenuated.

19. The microorganism for use according to any one of claims 1, 2 and 14 to 17, wherein the microorganism is a recombinant microorganism heterologously expressing the ATP hydrolyzing enzyme.

20. A (recombinant) bacterium comprising the nucleic acid as defined in any one of claims 8-10 for use in the treatment of dysbiosis or a dysbiosis-related disease.

21. The bacterium for use according to claim 1, 2 or 20, wherein the bacterium heterologously expresses the ATP hydrolyzing enzyme.

22. The bacterium for use according to any one of claims 1, 2, 20 or 21, wherein the bacterium is selected from Gram-positive bacteria, Gram-negative bacteria and Cyanobacteria.

23. The bacterium for use according to any one of claims 1, 2 and 20 to 22, wherein the bacterium is selected from the group consisting of Escherichia coli, Salmonella typhi, Salmonella typhimurium, Yersinia enterocolitica, Vibrio cholerae, Listeria monocytogenes, Lactococcus lactis and Shigella flexneri.

24. The bacterium for use according to claim 23, wherein the bacterium is E. coli of the strain Nissle 1917.

25. A viral particle comprising the nucleic acid as defined in any one of claims 8-10 for use in the treatment of dysbiosis or a dysbiosis-related disease.

26. The viral particle for use according to claim 1, 2 or 25, wherein the viral particle is a bacteriophage.

27. The ATP hydrolyzing enzyme, the nucleic acid, the host cell, the microorganism, the bacterium, or the viral particle for use according to any one of the previous claims, wherein dysbiosis is induced by an antibiotic agent, a chemotherapeutic agent, a diet or by maternal dysbiosis.

28. The ATP hydrolyzing enzyme, the nucleic acid, the host cell, the microorganism, the bacterium, or the viral particle for use according to any one of the previous claims, wherein the ATP hydrolyzing enzyme, the nucleic acid, the host cell, the microorganism, the bacterium, or the viral particle is comprised in a composition.

29. The ATP hydrolyzing enzyme, the nucleic acid, the host cell, the microorganism, the bacterium, or the viral particle for use according to claim 28, wherein the composition is a pharmaceutical composition further comprising a pharmaceutically acceptable carrier, diluent and/or excipient.

30. The ATP hydrolyzing enzyme, the nucleic acid, the host cell, the microorganism, the bacterium, or the viral particle for use according to any one of the previous claims, wherein the ATP hydrolyzing enzyme, the nucleic acid, the host cell, the microorganism, the bacterium, or the viral particle is administered via an enteral route of administration, preferably via oral administration.

31. The ATP hydrolyzing enzyme, the nucleic acid, the host cell, the microorganism, the bacterium, or the viral particle for use according to any one of the previous claims, wherein the dysbiosis-related disease is selected from inflammatory diseases, gastrointestinal tract-related disorders, metabolic disorders, CNS-related disorders, cancers and autoimmune diseases.

32. The ATP hydrolyzing enzyme, the nucleic acid, the host cell, the microorganism, the bacterium, or the viral particle for use according to any one of the previous claims, wherein the dysbiosis-related disease is selected from inflammatory bowel disease, irritable bowel syndrome, obesity, diabetes, metabolic syndrome, coeliac disease, colorectal cancer, Clostridioides difficile infection, autism spectrum disorder, urinary stone disease (USD), lupus erythematosus, rheumatoid arthritis, systemic sclerosis, Sjögren's syndrome, anti-phospholipid syndrome, cardiovascular syndrome, allergy, and asthma.

33. The ATP hydrolyzing enzyme, the nucleic acid, the host cell, the microorganism, the bacterium, or the viral particle for use according to any one of the previous claims, wherein the ATP hydrolyzing enzyme, the nucleic acid, the host cell, the microorganism, the bacterium, or the viral particle is administered in combination with a dysbiosis-inducing agent.

34. The ATP hydrolyzing enzyme, the nucleic acid, the host cell, the microorganism, the bacterium, or the viral particle for use according to claim 33, wherein the dysbiosis-inducing agent is an antibiotic; preferably selected from the group consisting of penicllins, tetracyclines, cephalosporins, quinolones, lincosamides, macrolides, sulfonamides, glycopeptides, aminoglycosides, carbapenems, ansamycins, carbacephems, lipopeptides, monobactams, nitrofurans, oxazolidinones, and polypeptides; more preferably the antibiotic is selected from the group consisting of vancomycin, ampicillin, metronidazole and cefoperazone.

35. The ATP hydrolyzing enzyme, the nucleic acid, the host cell, the microorganism, the bacterium, or the viral particle for use according to claim 33 or 34, wherein the dysbiosis-inducing agent is a chemotherapeutic agent; preferably selected from alkylating agents, anthracyclines, cytoskeletal disruptors, epothilones, histone deacetylase inhibitors, inhibitors of topoisomerase I or II, kinase inhibitors, nucleotide analogs and precursor analogs, platinum-based agents, retinoids, and vinca alkaloids and derivatives; for example the chemotherapeutic agent is 5-fluorouracil (5-FU).

36. A combination of
(i) a bacterium comprising a nucleic acid comprising a polynucleotide encoding an ATP hydrolyzing enzyme; and
(ii) a dysbiosis-inducing agent.

37. The combination of claim 36, wherein the encoded ATP-hydrolyzing enzyme is as defined in any one of claims 4 to 7.

38. The combination of claim 36 or 37, wherein the nucleic acid comprised in the bacterium is as defined in any one of claims 8-10.

39. The combination of any one of claims 36-38, wherein the bacterium is as defined in any one of claims 20-24.

40. The combination of any one of claims 36-39, wherein the dysbiosis-inducing agent is an antibiotic; preferably selected from the group consisting of penicllins, tetracyclines, cephalosporins, quinolones, lincosamides, macrolides, sulfonamides, glycopeptides, aminoglycosides, carbapenems, ansamycins, carbacephems, lipopeptides, monobactams, nitrofurans, oxazolidinones, and polypeptides; more preferably the antibiotic is selected from the group consisting of vancomycin, ampicillin, metronidazole and cefoperazone.

41. The combination of any one of claims 36-40, wherein the dysbiosis-inducing agent is a chemotherapeutic agent; preferably selected from alkylating agents, anthracyclines, cytoskeletal disruptors, epothilones, histone deacetylase inhibitors, inhibitors of topoisomerase I or II, kinase inhibitors, nucleotide analogs and precursor analogs, platinum-based agents, retinoids, and vinca alkaloids and derivatives; for example the chemotherapeutic agent is 5-fluorouracil (5-FU).

42. The combination of any one of claims 36-41, wherein the bacterium and/or the dysbiosis-inducing agent is/are comprised in a composition.

43. The combination of any one of claims 36-42 for use in medicine.

44. The combination of any one of claims 36-43 for use in the treatment of dysbiosis.

45. The combination for use of any one of claims 33-35, 43 and 44, wherein (i) the dysbiosis-inducing agent; and/or (ii) the ATP hydrolyzing enzyme, the nucleic acid, the host cell, the microorganism or the viral particle are administered repeatedly.

46. The combination for use of any one of claims 33-35 and 43-45, wherein (i) the dysbiosis-inducing agent; and (ii) the ATP hydrolyzing enzyme, the nucleic acid, the host cell, the microorganism or the viral particle are administered on the same day.

47. The combination for use of any one of claims 33-35 and 43-46, wherein the ATP hydrolyzing enzyme, the nucleic acid, the host cell, the microorganism or the viral particle is administered after administration of the dysbiosis-inducing agent.

48. A kit comprising:
(i) a bacterium comprising a nucleic acid comprising a polynucleotide encoding an ATP hydrolyzing enzyme; and
(ii) a dysbiosis-inducing agent.

49. The kit of claim 48, wherein the encoded ATP-hydrolyzing enzyme is as defined in any one of claims 4-7.

50. The kit of claim 48 or 49, wherein the nucleic acid comprised in the bacterium is as defined in any one of claims 8-10.

51. The kit of any one of claims 48-50, wherein the bacterium is as defined in any one of claims 20-24.

52. The kit of any one of claims 48-51, wherein the dysbiosis-inducing agent is an antibiotic; preferably selected from the group consisting of penicllins, tetracyclines, cephalosporins, quinolones, lincosamides, macrolides, sulfonamides, glycopeptides, aminoglycosides, carbapenems, ansamycins, carbacephems, lipopeptides, monobactams, nitrofurans, oxazolidinones, and polypeptides; more preferably the antibiotic is selected from the group consisting of vancomycin, ampicillin, metronidazole and cefoperazone.

53. The kit of any one of claims 48-52, wherein the dysbiosis-inducing agent is a chemotherapeutic agent; preferably selected from alkylating agents, anthracyclines, cytoskeletal disruptors, epothilones, histone deacetylase inhibitors, inhibitors of topoisomerase I or II, kinase inhibitors, nucleotide analogs and precursor analogs, platinum-based agents, retinoids, and vinca alkaloids and derivatives; for example the chemotherapeutic agent is 5-fluorouracil (5-FU).

54. The kit of any one of claims 48-53, wherein the bacterium and/or the dysbiosis-inducing agent is/are comprised in a composition.

55. The kit of any one of claims 48-54, wherein the kit further comprises a package insert or label with directions to treat dysbiosis or a dysbiosis-related disease by using a combination of (i) the dysbiosis-inducing agent and (ii) the bacterium comprising the nucleic acid comprising the polynucleotide encoding the ATP hydrolyzing enzyme.

56. The kit of any one of claims 48-55 for use in medicine, preferably in the treatment of dysbiosis.

57. A method for reducing the risk of occurrence, treating, ameliorating, or reducing dysbiosis or a dysbiosis-related disease in a subject in need thereof, comprising administering to the subject
(a) an ATP hydrolyzing enzyme,
(b) a nucleic acid comprising a polynucleotide encoding the ATP hydrolyzing enzyme,
(c) a host cell comprising the nucleic acid,
(d) a microorganism comprising the nucleic acid, or
(e) a viral particle comprising the nucleic acid.

58. A method for restoring or improving the balance of intestinal microbiota in a subject in need thereof, comprising administering to the subject
(a) an ATP hydrolyzing enzyme,
(b) a nucleic acid comprising a polynucleotide encoding the ATP hydrolyzing enzyme,
(c) a host cell comprising the nucleic acid,
(d) a microorganism comprising the nucleic acid, or
(e) a viral particle comprising the nucleic acid.

59. The method according to claim 58, wherein the microbiota balance is restored or improved during or after dysbiosis.


SEQUENCE LISTING (9 sequences)

SEQ ID NO: 1 - protein, 246 aa, Shigella flexneri
MKTKNFLLFCIATNMIFIPSANALKAEGFLTQQTSPDSLSILPPPPAEDSVVFLADKAHYEFGRSLRDANRVRLASEDAYYENFGLAFSDAYGMDISRENTPILYQLLTQVLQDSHDYAVRNAKEYYKRVRPFVIYKDATCTPDKDEKMAITGSYPSGHASFGWAVALILAEINPQRKAEILRRGYEFGESRVICGAHWQSDVEAGRLMGASVVAVLHNTPEFTKSLSEAKKEFEELNTPTNELTP

SEQ ID NO: 2 - protein, 246 aa, Artificial Sequence
  Loss-of-function isoform of apyrase
MKTKNFLLFCIATNMIFIPSANALKAEGFLTQQTSPDSLSILPPPPAEDSVVFLADKAHYEFGRSLRDANRVRLASEDAYYENFGLAFSDAYGMDISRENTPILYQLLTQVLQDSHDYAVRNAKEYYKRVRPFVIYKDATCTPDKDEKMAITGSYPSGHASFGWAVALILAEINPQRKAEILRRGYEFGESPVICGAHWQSDVEAGRLMGASVVAVLHNTPEFTKSLSEAKKEFEELNTPTNELTP

SEQ ID NO: 3 - DNA, 741 bp, Shigella flexneri
atgaaaaccaaaaactttcttcttttttgtattgctacaaatatgatttttatcccctcagcaaatgctctgaaggcagaaggttttctcactcaacaaacttcaccagacagtttgtcaatacttccgccgcctccggcagaggattcagtagtatttctggctgacaaagctcattatgaattcggccgctcgctccgggatgctaatcgtgtacgtctcgctagcgaagatgcatactacgagaattttggtcttgcattttcagatgcttatggcatggatatttcaagggaaaataccccaatcttatatcagttgttaacacaagtactacaggatagccatgattacgccgtgcgtaacgccaaagaatattataaaagagttcgtccattcgttatttataaagacgcaacctgtacacctgataaagatgagaaaatggctatcactggctcttatccctctggtcatgcatcctttggttgggcagtagcactgatacttgcggagattaatcctcaacgtaaagcggaaatacttcgacgtggatatgagtttggagaaagtcgggtcatctgcggtgcgcattggcaaagcgatgtagaggctgggcgtttaatgggagcatcggttgttgcagtacttcataatacacctgaatttaccaaaagccttagcgaagccaaaaaagagtttgaagaattaaatactcctaccaatgaactgaccccataa

SEQ ID NO: 4 - DNA, 17 bp, Artificial Sequence
  forward primer
  misc_feature (9)..(9): n is a, c, g, or t
cctacgggnggcwgcag

SEQ ID NO: 5 - DNA, 21 bp, Artificial Sequence
  reverse primer
gactachvgggtatctaatcc

SEQ ID NO: 6 - DNA, 840 bp, Artificial Sequence
  EcN malP gene portion
cgagcaggcacactggaagtattgctgcatcaggcgcagctttttaccggcagtatggttgtcgtttggatagagaactttggtcagtttttccgcgttgatgccctgctgttcggcacgcaggaaatcaccgtcgttaaatttagtcagatcaaacggatgcgcatgcgtcgcctgccacagacgcagtggctgcgccacgccattacgatagccgacaacggggagatcccacgcttgaccggtaatggtaaactccggctcccagcgtccatctttcgtcactttaccgccaatccctacctgcacatccagtgcttcgttgtggcggaaccacgggtagttaccgcgatgccagtcatccggcgcttcaacctgtttgccatcgacaaatgactggcggaacaagccatattgataattaaggccgtagccagtagctgactgcccgacagttgccattgagtcgaggaagcacgccgccagacgtcccagaccaccgttccccagcgccgggtcgatctcttcttccaacaggtcagtcaggttgatgtcataagccttcaacgaatcctgtacatcctgataccagccgagattcaacaggttgttgcccgtcaggcgaccaatcaaaaactccattgagatgtagttaacatgtcgctgattcgccactggcttggcgaatggctgagcacgcagcatttcggccagtgcttcgctcactgccagccaccactggcgaggagtcatttcagccgcagaatttaagccataacgctgccactgacgtgaaagcgcttcctgaaattgcttatcgttaaaaataggttgtgacat

SEQ ID NO: 7 - DNA, 813 bp, Artificial Sequence
  EcN malT gene portion
atgctgattccgtcaaaattaagtcgtccggttcgactcgaccataccgtggttcgtgagcgcctgctggctaaactttccggcgcgaacaacttccggctggcgctgatcacaagtcctgcgggctacggaaagaccacgctcatttcccagtgggcggcaggcaaaaacgatatcggctggtactcgctggatgaaggtgataaccagcaagagcgtttcgccagctatctcattgccgccgtgcaacaggcaaccaacggtcactgcgcgatatgtgagacgatggcgcaaaaacggcaatatgccagcctgacgtcactcttcgcccagcttttcattgagctggcggaatggcatagcccactttatctggtcatcgatgactatcatctgatcactaatcctgtgatccacgagtcaatgcgcttctttattcgccatcaaccagaaaatctcacccttgtggtgttgtcacgcaaccttccgcaactgggcattgccaatctgcgtgttcgtccagctagcgaattcgctggaaattggcagtcagcaactggcatttacccatcaggaagcgaagcagttttttgattgccgtctgtcatcgccgattgaagctgcagaaagcagtcggatttgtgatgatgtttccggttgggcgacggcactgcagctaatcgccctctccgcccggcagaatactcactcagcccataagtcggcacgccgcctggcgggaatcaatgccagccatctttcggattatctggtcgatgaggttttggataacgtcgatctcgcaacgcgcca

SEQ ID NO: 8 - DNA, 1285 bp, Artificial Sequence
  DNA fragment including PproD promoter, BBa_BB0032 RBS, S. flexneri phoN2 gene and phoN2 transcriptional terminator
cagctaacaccacgtcgtccctatctgctgccctaggtctatgagtggttgctggataactttacgggcatgcataaggctcgtataatatattcagggagaccacaacggtttccctctacaaataattttgtttaacttttactagagtcacacaggaaagtactagatgaaaaccaaaaactttcttcttttttgtattgctacaaatatgatttttatcccctcagcaaatgctctgaaggcagaaggttttctcactcaacaaacttcaccagacagtttgtcaatacttccgccgcctccggcagaggattcagtagtatttctggctgacaaagctcattatgaattcggccgctcgctccgggatgctaatcgtgtacgtctcgctagcgaagatgcatactacgagaattttggtcttgcattttcagatgcttatggcatggatatttcaagggaaaataccccaatcttatatcagttgttaacacaagtactacaggatagccatgattacgccgtgcgtaacgccaaagaatattataaaagagttcgtccattcgttatttataaagacgcaacctgtacacctgataaagatgagaaaatggctatcactggctcttatccctctggtcatgcatcctttggttgggcagtagcactgatacttgcggagattaatcctcaacgtaaagcggaaatacttcgacgtggatatgagtttggagaaagtcgggtcatctgcggtgcgcattggcaaagcgatgtagaggctgggcgtttaatgggagcatcggttgttgcagtacttcataatacacctgaatttaccaaaagccttagcgaagccaaaaaagagtttgaagaattaaatactcctaccaatgaactgaccccataaagctggacagcctgtatcaggctatggagggcccatagacaaatctaccctatatgagcataggaggagtctatgggcacaccacgttttacccctgaatttaagggattactggaaaggctgggacatatccctccggcagaagcagaaaaagcttattatgctgccatcggaaacgatgatctggcaacctgagttcacagataaaacattctctaggaaactcggggcggttccgttcaccacatgcaatgtggtgttgcaggggaacggtctgcccatcccctatgtcgatcaatataacagaaatgacaacttcagattcagggcacaacctaaatatattttaggtcacctctcaaatcgtttgcctga

SEQ ID NO: 9 - DNA, 995 bp, Artificial Sequence
  DNA fragment including the E. coli cat gene flanked by the FRT sequences
gaagttcctattctctagaaagtataggaacttcggcgcgcctacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaggcgcgccatttaaatgaagttcctattccgaagttcctattctctagaaagtataggaacttcgaagcagctccagcctacacaatgaattc